(19) United States
(12) Patent Application Publication
(10) Pub. No.: US 2007/0239003 A1
(43) Pub. Date: Oct. 11, 2007

(54) APPARATUS AND METHODS FOR ACOUSTIC DIAGNOSIS

(76) Inventors: Hemchandra M. Shertukde, Simsbury, CT (US); Rekha Shertukde, Simsbury, CT (US); Peter V. Beckmann, Hartford, CT (US); Raymond McLaughlin, Hartford, CT (US); Abhilash R. Menon, Hartford, CT (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/618,515

(22) Filed: Dec. 29, 2006

Related U.S. Application Data
(63) Continuation of application No. 11/333,791, filed on Jan. 17, 2006.
(60) Provisional application No. 60/645,284, filed on Jan. 20, 2005.
(60) Provisional application No. 60/654,840, filed on Feb. 17, 2005.
(60) Provisional application No. 60/671,954, filed on Apr. 15, 2005.
(60) Provisional application No. 60/699,812, filed on Jul. 14, 2005.

(51) Int. Cl. A61B 8/00

(52) U.S. Cl. 600/437; 600/463

(57) ABSTRACT

The apparatus and methods disclosed herein relate to diagnosis of disease through the detection of signals from portions of a body. The signals may be acoustic signals, which can be used to diagnose the presence, severity and/or location of occlusions in arteries, such as the coronary arteries. The signals may be detected through noninvasive methods such as, for example, passive reception. Such methods can avoid many of the problems associated with invasive angiogram and angioplasty procedures. The apparatus and methods described herein are not limited to use for diagnosing occlusions in the coronary arteries, but can be used for a wide variety of biomedical diagnosis in human and nonhuman animals.

### CROSS-REFERENCE TO RELATED APPLICATIONS

This application is a continuation of U.S. patent application Ser. No. 11/333,791, filed Jan. 17, 2006, entitled “APPARATUS AND METHODS FOR ACOUSTIC DIAGNOSIS,” which is hereby incorporated by reference herein in its entirety and made a part of this specification. U.S. patent application Ser. No. 11/333,791 claims priority to the following patent applications, and the present application incorporates each of these applications herein by reference in their entirety and makes each a part of the specification hereof: U.S. Provisional Patent Application No. 60/645,284, filed on Jan. 20, 2005, entitled “APPARATUS AND METHOD FOR NON-INVASIVE DIAGNOSING OF CORONARY ARTERY DISEASE”; U.S. Provisional Patent Application No. 60/654,840, filed on Feb. 17, 2005, entitled “APPARATUS AND METHOD FOR NON-INVASIVE DIAGNOSING OF CORONARY ARTERY DISEASE”; U.S. Provisional Patent Application No. 60/671,954, filed on Apr. 15, 2005, entitled “APPARATUS AND METHOD FOR NON-INVASIVE DIAGNOSING OF CORONARY ARTERY DISEASE”; and U.S. Provisional Patent Application No. 60/699,812, filed on Jul. 14, 2005, entitled “NON-INVASIVE TOOL FOR CORONARY ARTERY DIAGNOSIS USING SIGNAL CHARACTERISTIC ANALYSIS (CADSCAN) AND ISO-SURFACE OPTIMAL MEMBRANE-ADHERENT COMPLIANT (ISOMAC) SENSORS.” This application also incorporates herein by reference the following application in its entirety and makes it a part of the specification hereof: U.S. patent application Ser. No. 10/830,719, filed on Apr. 23, 2004, entitled “APPARATUS AND METHOD FOR NON-INVASIVE DIAGNOSING OF CORONARY ARTERY DISEASE.”

### BACKGROUND

### Field of the Invention

The inventions disclosed herein relate generally to devices and methods for sensing and processing signals from a body and specifically to devices and methods for sensing and processing acoustic signals from a body.

### SUMMARY

The apparatus and methods disclosed herein relate to diagnosis of disease through the detection of signals from portions of a body. The signals may be acoustic signals, which can be used to diagnose the presence, severity and/or location of occlusions in arteries, such as the coronary arteries. The signals may be detected through noninvasive methods such as, for example, passive reception. Such methods can avoid many of the problems associated with invasive angiogram and angioplasty procedures. The apparatus and methods described herein are not limited to use for diagnosing occlusions in the coronary arteries, but can be used for a wide variety of biomedical diagnosis in human and nonhuman animals.

### BRIEF DESCRIPTION OF THE DRAWINGS

The foregoing and other objects and advantages of the present inventions will be further explained in the detailed description of embodiments in connection with the accompanying drawings wherein throughout the figures, like reference numerals describe like elements.

FIG. 1 is an illustration of a human heart showing coronary arteries.

FIG. 2 is a cross-sectional illustration of a coronary artery that has been partially narrowed by fissuring plaque. Insets show partial and complete occlusive thrombosis.

FIG. 3 schematically illustrates a catheter device inserted for an angioplasty or angiogram procedure on a patient.

FIGS. 4A and 4B schematically illustrate fluid flow in a pipe having a narrow portion. FIG. 4A schematically illustrates laminar flow, and FIG. 4B schematically illustrates turbulent flow downstream of the narrow portion.

FIG. 5 illustrates a Morlet mother wavelet.

FIGS. 6A-6D illustrate daughter wavelets of the Morlet mother wavelet shown in FIG. 5.

FIG. 7 schematically illustrates a sampling grid for a discrete wavelet transform and shows examples of daughter wavelets corresponding to four time-frequency resolution cells.

FIG. 8 schematically illustrates a Cartesian coordinate system that is convenient for showing a stenosis located at (xₛ,yₛ,zₛ), sound waves emitted by the stenosis, and a plurality of acoustic sensors located at (xᵢ,yᵢ,zᵢ), that receives the sound waves.

FIG. 9 is a flow chart showing general steps in a method for diagnosing coronary artery occlusions.

FIG. 10A schematically illustrates the general correspondence between acoustic sensors and the underlying anatomy of the body on which the sensors are placed.

FIG. 10B schematically illustrates an embodiment for placement of acoustic sensors on the body of the patient.

FIG. 10C schematically illustrates an embodiment of a template for placement of acoustic sensors on the body of a patient.

FIGS. 11A-11C illustrate various heart signals and features present in heart signals.

FIG. 12A is a schematic block diagram illustrating a method for diagnosing the presence of a coronary artery occlusion from acoustic data.

FIG. 12B is a plot of the absolute value of wavelet coefficients from a diastolic portion of a heartbeat for six different values of a scale parameter.

FIG. 13 is a flowchart illustrating a method for statistically correlating a wavelet diagnostic parameter with a comparison diagnostic parameter.

FIG. 14A schematically illustrates an embodiment of an apparatus for diagnosing coronary artery disease

FIG. 14B schematically illustrates a plan view of an embodiment of an apparatus for diagnosing coronary artery disease.

FIG. 14C schematically illustrates a side view of the embodiment shown in FIG. 14B.

FIG. 14D is a photograph of an embodiment of an apparatus for diagnosing coronary artery disease.

FIG. 14E schematically illustrates a front perspective view of an embodiment of a diagnostic device comprising a portable unit and a base station.

FIG. 14F schematically illustrates a back perspective view of the diagnostic device shown in FIG. 14E.

FIG. 15A schematically illustrates general electronic architecture that can be used for embodiments of a diagnosing device.

FIGS. 15B and 15C schematically illustrate functional block diagrams for embodiments of an apparatus for diagnosing coronary artery disease.

FIGS. 16A-16N schematically illustrate electronics suitable for use in an apparatus for diagnosing coronary artery disease.

### DETAILED DESCRIPTION OF PREFERRED EMBODIMENTS

Body organs and tissues can generate acoustic energy that is transmitted through portions of the body. The acoustic energy can comprise sound waves, which are oscillations of the medium through which the acoustic energy travels. Often the medium is compressed in the same direction as the propagation of the sound wave, forming a compression wave. The sound waves propagate at the speed of sound, which depends in part on the medium through which the sound waves travel. For example, the speed of sound in soft body tissue can be about 1540 m/s, while the speed of sound in bone tissue can be about 4000 m/s. The sound waves generated by the body can relate to the movement of cells or bodily fluids within the various organs or regions of the body, the movement of muscles, the intake of air into the lungs, etc. Each of these sounds can contain information that can be used by a doctor for diagnostic purposes. For example, sounds produced by the pumping heart, the opening and closing of heart valves, and/or the flow of blood through the vasculature can provide information about the health status of a patient relating to the heart and vasculature.

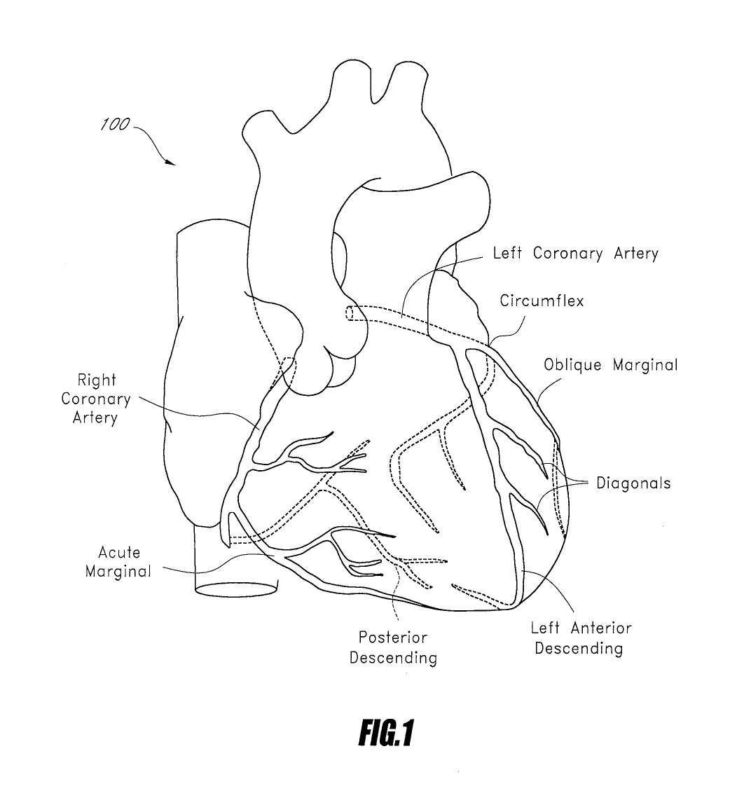

Many patients suffer from a dangerous medical condition wherein cholesterol or plaque deposits build up on the interior walls of bodily vessels or arteries. Coronary artery disease refers to such a build-up when it occurs on the interior walls of the arteries of the heart. FIG. 1 illustrates a heart 100 and the principle coronary arteries. The build-up of plaque or cholesterol can contribute to a blockage of the arteries, and when a severe blockage occurs in the coronary artery, a fatal heart attack can result. Medical diagnosis is possible using acoustic signals produced within the body when the biophysical processes creating the sounds are understood.

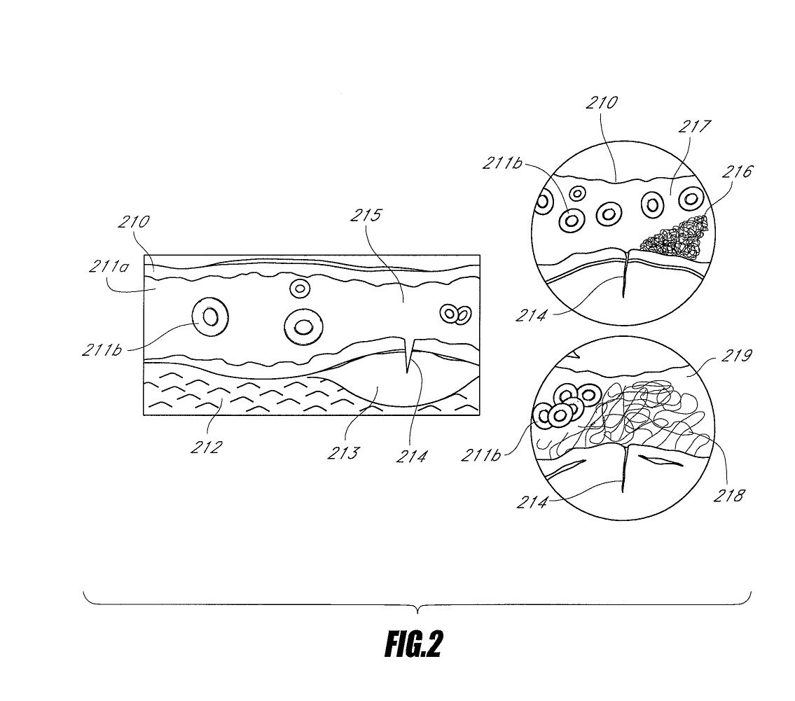

FIG. 2 illustrates an artery 210 that carries blood plasma 211a and red blood cells 211b (erythrocytes) throughout the body of the patient. In a patient with coronary artery disease, a layer of cholesterol or plaque 212 is formed on the interior walls of the artery 210. This layer of plaque 212 reduces the capacity of the affected arteries to carry blood, thereby reducing the flow of blood through the arteries and the amount of blood delivered to tissue (e.g., muscle tissue) supplied by the arteries, leading to loss of nutrients and oxygen to the tissue. The layer of plaque 212 can also weaken the walls of affected arteries. As shown in FIG. 2, plaque buildup 213 may cause a narrowing 215 of the artery 210. The narrowing 215 of an artery may be referred to herein as a stenosis or as an occlusion. As used herein, the terms “stenosis” and “occlusion” are broad terms, and are used in their ordinary senses, and include, without limitation except as explicitly stated, any abnormal narrowing, blockage (either partial or total), or decrease in cross-sectional area of a blood vessel or tubular organ. The terms “stenosis” and “occlusion” include, but are not limited to, narrowing or blockage of the coronary arteries caused by coronary artery disease. The terms “stenosis” and “occlusion” are used interchangeably except as specifically stated.

A crack 214 may develop in the plaque 212 and cause a blood clot (thrombus) to form in an artery 210. The upper inset in FIG. 2 schematically illustrates a wall thrombus 216 that has caused a partial occlusion 217 (e.g., a cross-sectional area of the artery 210 is reduced). Partial occlusions 217 may give rise to unstable angina in some patients and may be prone to embolization, which can cause additional occlusions downstream of the original occlusion 217. The lower inset in FIG. 2 schematically illustrates a thrombus 218 that has caused a complete occlusion 219 (e.g., the cross-sectional area of the artery 210 is substantially completely blocked). Complete occlusions 219 may give rise to myocardial infarction in some patients. Blood clots (such as the example clots 216 and 218 shown in FIG. 2) can compound the problem caused by plaque build-up. Not only does the plaque 212 at least partially occlude the vessel or artery, but the blood clot can lodge in the occluded portion of the vessel and further compound the blockage problem. Of course, the blood clot itself can embolize and fully or partially occlude other vessels or arteries of the body, even if not at the same location in the vasculature where the plaque build-up has occurred. Blood clots in the coronary arteries are the cause of many heart attacks.

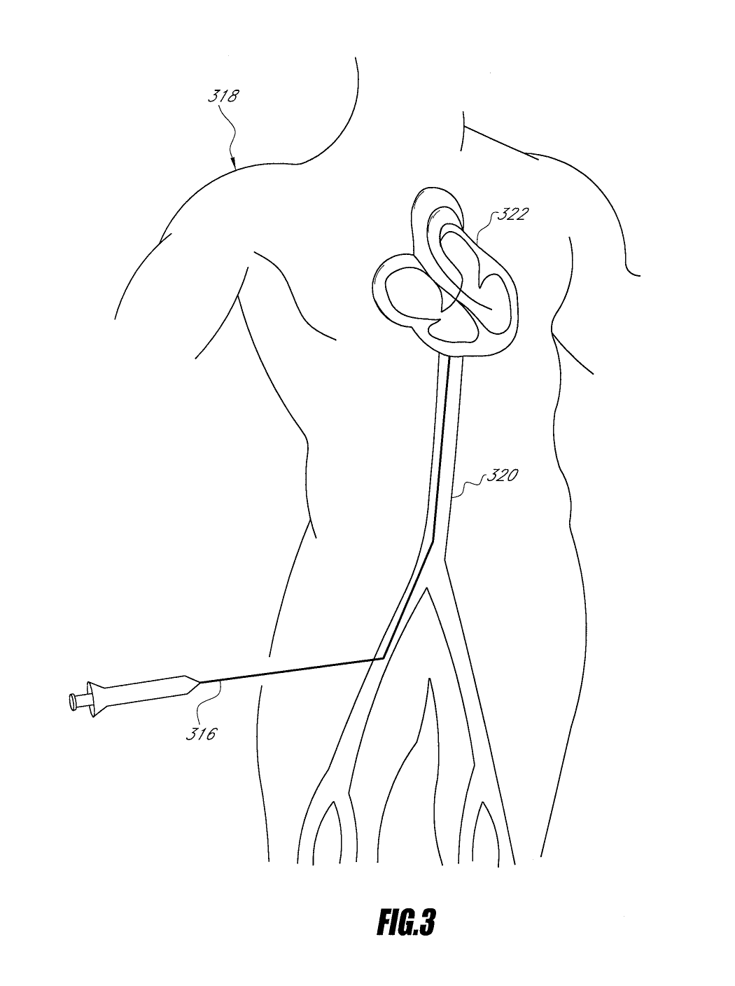

Currently, many invasive techniques are used to diagnose coronary artery disease. For example, an “angiogram” is a highly invasive procedure requiring a catheter to be inserted into the body (through a femoral or other large artery, for example). The catheter is then fed through the vasculature until it reaches the vessel or artery to be examined, for example a coronary artery. This procedure has many disadvantages related to its highly invasive nature and can create risks of very harmful side effects. FIG. 3 illustrates the length of catheter that can be required for an angiogram. A catheter 316 is shown schematically as it extends from an insertion point in the femoral artery of a patient 318, through a blood vessel 320 and into the patient's heart 322. Such a catheterization can be necessary for angiogram and subsequent angioplasty procedures. Angioplasty involves the treatment of occlusions in the vessel by compressing, removing, or otherwise altering the shape of plaque deposits on the side of a vessel. Because of the highly invasive nature of these methods of diagnosing coronary artery disease, a great need exists for a noninvasive diagnosis apparatus method such as described herein.

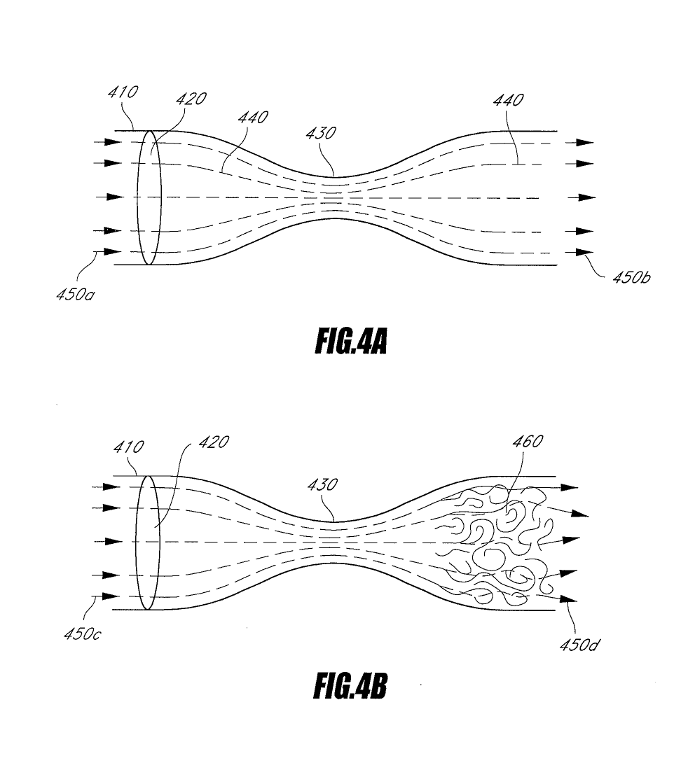

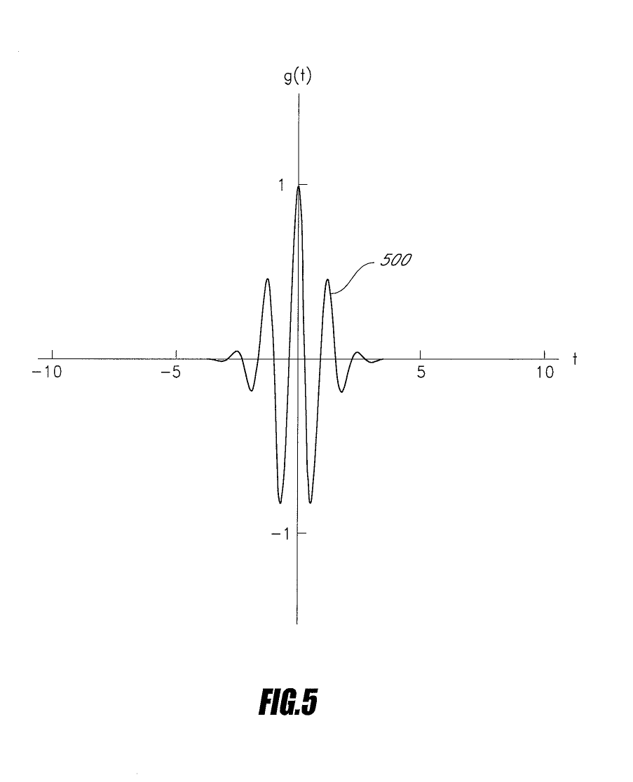

As described above, coronary artery disease can cause a narrowing of the passages through which blood flows. Although many biological systems are more complex, a simplified model can be useful to help describe the physics of the fluid flow through a passageway that has a relatively narrow portion. FIGS. 4A and 4B schematically illustrate a passageway 410 having a cross-sectional area 420 that varies along its length. The cross-sectional 420 area decreases to a minimum value at a constriction region 430 and then increases to its original value. A pump (e.g., the heart shown in FIG. 1) provides a steady flow of fluid through the passageway 410. FIG. 4A schematically illustrates a condition of laminar flow in which the fluid speeds are steady, smooth, and regular. Fluid flow lines 440, which indicate the paths taken by portions of the fluid, smoothly converge as the constriction 430 is approached and smoothly diverge after the constriction 430 is passed. The flow speed smoothly increases from an initial value (indicated by arrows 450a), reaches a maximum value at the position of the constriction 430, and the smoothly decreases back to the initial value (indicated by arrows 450b). Laminar flow typically occurs only at lower flow speeds, and any sound produced by generally laminar flow is minimal. FIG. 4B schematically illustrates a condition of turbulent flow, which typically occurs when fluid speeds are higher than found in laminar conditions. The flow speed smoothly increases from an initial value (indicated by arrows 450c) that is similar to the initial value 450a in FIG. 4A, and the flow speed increases as the constriction 430 is approached. However, in contrast to the laminar flow in FIG. 4A, the turbulent flow in regions 460 past the constriction 430 is characterized by flow speeds 450d that are unsteady, irregular, and chaotic. The fluid pressure in the turbulent portions 460 of the passageway 410 is increased, which makes it more difficult for the pump to maintain the flow rate. The turbulent flow generates stresses on the walls of the passageway 410, which can lead to damage. The turbulent flow breaks into an irregular pattern of turbulent eddies in the turbulent region 460, which may be contrasted with the smooth laminar flow lines 440 shown in FIG. 4A. The turbulent flow generates distinctive sound waves, which propagate away from the turbulent region 460. The sounds generated by turbulent flow are generally louder (e.g., have higher acoustic amplitude) and higher frequency than sounds generated by laminar flow. For example, the turbulent flow and/or other physiological phenomena associated with a heart murmur may be heard through a stethoscope.

In comparison with the simplified model of FIGS. 4A and 4B, the occlusion in a coronary artery is generally less smooth and gradual than the narrow portion 430 of the model passageway 410, which can lead to a more complex turbulent flow than depicted in the model of FIG. 4B. For example, FIG. 2 schematically illustrates several occlusions of a blood vessel due to plaque cracking, fissuring, and rupturing. The occlusions 217, 219 may be highly irregular in structure and may have differing degrees of porosity. Additionally, the occlusions 217, 219 may include an irregularly shaped thrombus 216, 218. Further, in the body, the blood flow does not necessarily occur at a constant velocity, even in portions of a blood vessel with a constant cross-sectional area, because the heart pumps with periodic pulses rather than a steady, continuous force in the direction of flow. Also, not only can the vessels and arteries expand outwardly under pressure, but they can also be constricted by the muscles around them. These biological factors reduce the likelihood that a coronary artery will have a constant cross-sectional area. However, as noted above, plaque build-up can reduce the elasticity of the coronary arteries, reducing this effect in a patient with coronary artery disease.

Turbulence caused by the narrowing or occlusion of the fluid passageway that occurs from plaque build-up in a coronary artery generates acoustic energy. This turbulence is generally not present, or at least not to the same degree, in a healthy patient having no arterial occlusion, thus the presence or absence of such turbulence, as well as its other characteristics, can be used in diagnosing coronary artery disease. The turbulence can be especially strong past the occlusion, such as on the downstream side of the occlusion in the fluid passageway (e.g., the region 460 in FIG. 4B). The fluid turbulence can generate high frequency sounds or acoustic signals. These signals can be detected during the portion of the heart's pumping cycle referred to as the diastolic period. This portion of the heart's cycle is relatively quiet because the muscles of the heart are not strongly contracting and the heart valves are relatively quiet compared to other portions of the heart's pumping cycle.

The acoustic energy produced by the body organs, such as a heart, may be detected and monitored noninvasively, by one or more acoustic sensors placed on the outside of the body. The sensors can produce an analog signal with an amplitude corresponding to the amplitude of the incoming sound wave when that sound wave arrives at the acoustic sensor. An acoustic energy or acoustic intensity is proportional to the square of the sound wave amplitude. The analog signal thus detected can be a continuously varying function of time, and it can be transmitted to hardware or software modules for processing. Generally, the analog signal is converted to a digital signal by sampling the analog signal at a set of discrete times. An analog-to-digital converter (ADC) may be used for this conversion. The digital signal comprises a set of values of the acoustic signal at the sampling times. Typically, the analog signal is sampled at a fixed sampling rate, e.g., 22,000 Hz. The sampling rate can be adjusted or tuned, depending on the frequency of the signal to be processed and/or the frequency or other characteristics of any noise. The sampling rate may depend on the processing speed of other components in the apparatus.

Analysis of an Acoustic Signal

In some embodiments, apparatus and methods for detecting an occlusion in a coronary artery of a patient is provided. The apparatus can have one or multiple acoustic sensors that attach to the body of a patient (e.g., on the patient's chest at known locations). The sensors can receive acoustic signals generated by the body and can communicate the acoustic signals to another portion of the apparatus for analysis, for example, by generating an electric signal that is proportional to the acoustic signal. A threshold amplitude range or frequency range or temporal range may be established for identifying the signals to be evaluated. The signals can be processed to determine the presence and/or the severity of an occlusion or occlusions in a coronary artery. In some embodiments, the method further includes determining a location of an occlusion relative to the location of one or more of the acoustic sensors or relative to the anatomy of the heart.

The signal processing methods may include amplifying, filtering, digitizing, synchronizing, and/or multiplexing the signals. The processing can further include identifying a portion of the signal that corresponds to a heartbeat or to a diastolic portion of the heartbeat. In some embodiments, the processing methods may identify an event that indicates a beginning to a systolic portion and/or a diastolic portion of the heartbeat. In certain embodiments, the event may comprise a portion of the acoustic signal that is within a predetermined frequency range and/or that exceeds a threshold amplitude. Acoustic signals having certain frequencies and exceeding a threshold amplitude may indicate the existence of an occlusion in one or more coronary arteries.

The signal processing methods can further include transforming various combinations of the signals received from the acoustic sensors. The transform can include a Fourier transform, a wavelet transform, or other signal analysis transform. In some embodiments, more than one transform may be applied to the signals. The wavelet transform analysis can provide time delay and scale (frequency) analysis of the signals. The time delay parameters and the scale parameters may be used to estimate the time taken by heart sounds (or sounds originating from turbulence inside coronary arteries) to travel through the body and be detected by the acoustic sensors. In some embodiments, relative time delays may be evaluated to determine the location of the occlusion in one of the coronary arteries. A value of the time delay and scale parameter where a wavelet transform parameter has a local maximum may be identified and may be used to determine the severity of the occlusion. In other embodiments, a centroid of a portion of the wavelet coefficients may be used to determine time delays. In other embodiments, a variance of the wavelet coefficients may be used to indicate the presence or severity of coronary artery disease.

Certain embodiments of the methods disclosed herein can include some or all of the following: attaching a plurality of acoustic sensors to the chest of a patient; receiving a signal from each of the plurality of acoustic sensors, the signals representing a plurality of heartbeats of the patient; establishing a threshold amplitude and a frequency range for identifying the signals to be evaluated; and processing the signals for determining the presence or severity of an occlusion in a coronary artery and a location of the occlusion relative to the locations of the plurality of acoustic sensors.

In some embodiments, the method for detecting an occlusion in a coronary artery of a patient can include one or more of the following as part of signal processing: amplifying, digitizing, filtering, synchronizing, and/or multiplexing the signals. In some embodiments, a method for detecting an occlusion in a coronary artery of a patient can include identifying the existence of an amplitude of the signals exceeding the established threshold amplitude that is within the established frequency range as part of the processing step. In certain embodiments, the processing step of the method can further comprise conducting a wavelet transform analysis on at least one of the signals received from the plurality of acoustic sensors. The wavelet transform analysis can provide either a time domain analysis or a frequency analysis, or both. In some embodiments, the method for detecting an occlusion in a coronary artery of a patient can further comprise displaying at least one of a location of the obstruction relative to a location of at least one of the plurality of acoustic sensors, or relative to a visualization of the patient's heart, or by a description in text. Furthermore, the method can include displaying information relating to the severity of the occlusion.

In some embodiments, the method for detecting an occlusion in a coronary artery of a patient can further include attaching the acoustic sensors at known locations relative to a reference point identified on the patient's chest. The location of an occlusion can then be identified relative to the locations of the acoustic sensors and/or the reference point.

Signal Processing with Wavelet Transforms

As described above, acoustic sensors receive an acoustic signal corresponding to sound waves emitted by the heart (or to sound waves generated by arterial turbulence). The acoustic signal represents an amplitude of sound waves reaching positions of the sensors. In certain embodiments, the acoustic sensors respond to the sound waves by generating an analog received signal. In certain such embodiments, the analog received signal may be amplified, filtered, sampled and digitized, for example, by an analog-to-digital converter, to produce a digital signal. The digital signal is representative of the amplitude of the sound waves emitted by the heart and received by the sensors at the sampling times. The digital signal may be processed using many known signal processing techniques. For example, in some embodiments, the digital signal may be further filtered to remove unwanted or extraneous signal components such as ambient acoustic noise. Additionally, various transforms may be applied to the digital signal, either before or after filtering. For example, a Fourier transform may be applied to the digital signal to determine the amounts of acoustic energy in sound waves oscillating at different frequencies.

In methods applying Fourier analysis, the digital signal is decomposed as a weighted sum of sinusoidal basis functions (sines and cosines), each of which oscillates at a different, constant frequency. The amplitude of the sinusoidal basis functions does not decay in time, which means that the basis functions have infinite extent in the time domain. Fourier analysis may be used to calculate how much acoustic energy (or power) is contained in the signal at each different frequency.

It has been found that coronary artery occlusions can generate acoustic energy in the frequency range from about 500 Hz to about 1000 Hz. See, e.g., J. L. Semmlow, et al., “Noninvasive Detection of Coronary Artery Disease Using Parametric Spectral Analysis Methods,” pp. 33-35, IEEE Engineering in Medicine and Biology Magazine, March 1990, and Y. M. Akay, et al., “Noninvasive Acoustical Detection of Coronary Artery Disease: A Comparative Study of Signal Processing Methods,” pp. 571-578, IEEE Transactions on Biomedical Engineering, vol. 40, no. 6, June 1993, both of which are hereby incorporated by reference herein in their entirety and made a part of this specification. The amplitude and the frequency range corresponding to the turbulent acoustic energy may be different in different patients, and the aforementioned range from about 500 Hz-1000 Hz is intended to serve only as an example of the acoustic frequency range that may be useful for diagnostic purposes. For example, the frequency range may be from about 300 Hz to 2000 Hz in some patients. Other frequencies, higher and/or lower, may be generated by coronary artery occlusions, and these frequencies may be different in nonhuman animals. Further, acoustic energy emitted by other types of abnormality or diseased portions of the body may comprise a different frequency range.

A signal may be characterized by a central frequency and a bandwidth. The central frequency represents an average frequency in the signal, whereas the bandwidth represents a frequency range that includes most of the acoustic energy. The central frequency and the bandwidth may be determined using Fourier analysis techniques. Signals may be classified as narrowband or wideband depending on the fractional bandwidth, which is a ratio of the bandwidth to the central frequency. Narrowband signals have a fractional bandwidth smaller than one, which means that most of their energy is present in a narrow band surrounding the central frequency. In contrast, wideband signals have a fractional bandwidth greater than one, which means that a substantial portion of their energy is present at frequencies away from the central frequency.

Additionally, signals may be characterized as being stationary or non-stationary. A stationary signal has constant or slowly varying statistical attributes such that a snapshot of the signal at a particular time is likely to show similar statistical attributes as a snapshot taken at another time. A non-stationary signal may have a random component, so that a snapshot of a signal at a particular time may seem to have very little correspondence to a snapshot of that same signal taken at a different time.

Because the sinusoidal basis functions used in Fourier analysis oscillate at constant frequencies and do not decay with time, Fourier methods may be suitable for narrowband, stationary signals. The acoustic energy emitted by the heart, however, may comprise a wideband, non-stationary signal. For example, the fractional bandwidth measured from a heart signal from one patient was found to be equal to about 2.2, which is larger than one (the point demarcating the transition from narrowband to wideband). Furthermore, blood flow in occluded arteries is known to be characterized by turbulence, which is generally a random, non-stationary process. Accordingly, signal analysis techniques suitable for wideband, non-stationary acoustic signals may be useful for analyzing heart signals. Such techniques may be used in conjunction with Fourier methods or other signal analysis techniques as well.

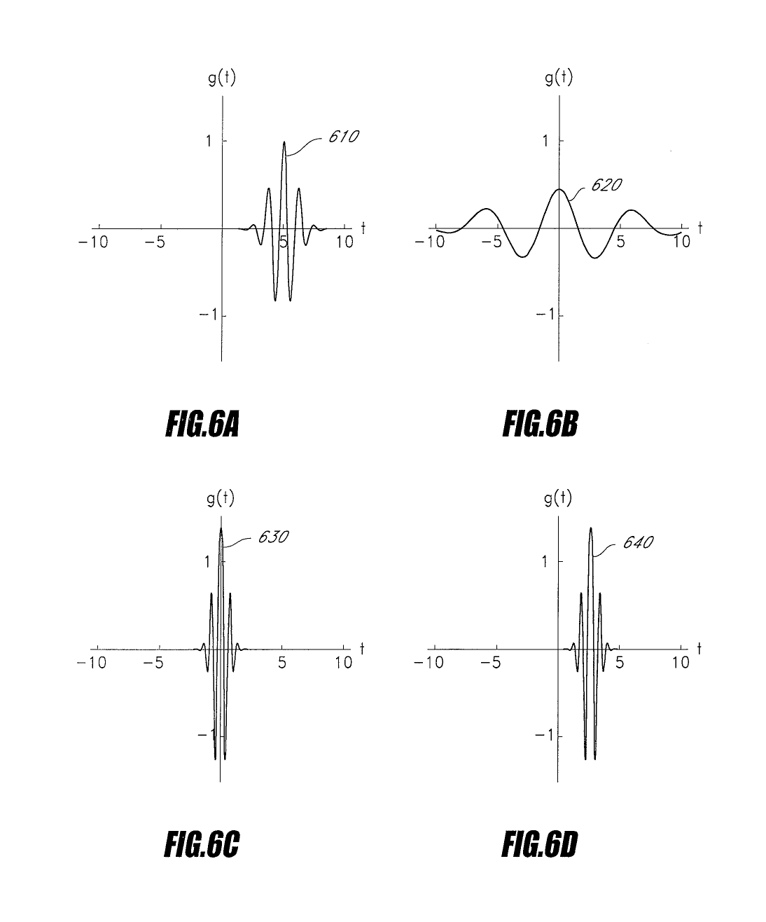

Wavelet analysis was developed in part to provide analysis methods for wideband, non-stationary signals. In a wavelet transform, a signal is decomposed in terms of basis functions called wavelets. In contrast to the sinusoidal Fourier basis functions having infinite extent, wavelets are localized around a central time, and their amplitude is small for times earlier or later than the central time. Wavelets, like the Fourier basis functions, are oscillatory, but wavelets do not generally oscillate at a fixed frequency. FIG. 5 shows a representative example of a wavelet 500, which is known as the “Morlet wavelet.” FIG. 5 is a plot of the amplitude of the Morlet wavelet 500 as a function of time t. The Morlet wavelet 500 is oscillatory, centered around the central time t=0, and decays in amplitude away from t=0.

Each of the wavelet functions used in a wavelet transform is derived from a single “mother wavelet” (also called an “analyzing wavelet”). Wavelets derived from the mother wavelet are called “daughter wavelets.” A daughter wavelet is derived from the mother by (i) translating (shifting) the mother wavelet in time and (ii) scaling (dilating or compressing) the mother wavelet in amplitude. Accordingly, daughter wavelets are translated and scaled replicas of the mother wavelet. The wavelet transform of a signal is a mathematical microscope that measures how well the signal correlates with the daughter wavelet at each value of translation and scale. In effect, by adjusting the translation and scale parameters, the wavelet transform permits one to change the focus of the mathematical microscope and to resolve the details of the signal at different times and at different frequencies. Additional details about wavelet transforms can be found in many commonly available textbooks such as “Wavelet Theory and Its Applications,” by Randy K. Young, Kluwer Academic Publishers, which is hereby incorporated by reference herein in its entirety for all that it discloses and is made a part of this specification.

An advantage of wavelet analysis is that there is a very large variety of potential mother wavelet functions, in contrast to Fourier analysis, which generally requires less diverse, highly periodic functions such as sines and cosines. By selecting suitable mother wavelets, different mathematical aspects of the signal can be analyzed. For example, the Morlet wavelet 500 shown in FIG. 5 may be suitable for determining whether a signal contains short duration “bursts” of wave energy. Because the mother wavelet is a function that is localized in time and may be suitably scaled and translated, wavelet transforms are appropriate for analyzing wideband, non-stationary signals such as those from the heart.

The value of the mother wavelet as a function of the time t will be denoted by the function g(t). For example, the Morlet mother wavelet 500 shown in FIG. 5 may be represented by the mathematical function
e^{-t}^{²}^{/2}[cos(σt)−e^{-σ}^{²}^{/2}], (1)
where σ is an adjustable parameter (set to be equal to five in FIG. 5). A daughter wavelet is a scaled and translated replica of the mother wavelet. The time translation parameter will be denoted by τ, and the scale parameter will be denoted by s. Larger values of τ correspond to larger translations of the mother wavelet in time. Larger values of s correspond to longer time scales and lower frequencies. Smaller values of s correspond to shorter time scales and higher frequencies. Accordingly, the oscillation frequency of a daughter wavelet is inversely proportional to its scale parameter. The scaled and translated daughter wavelet is denoted by g_{s,τ}(t) and is defined by the following relation:

$${g}_{s , τ} ( t ) = \frac{1}{\sqrt{\vert s \vert}} g ( \frac{t - τ}{s} ) . \qquad ( 2 )$$

The normalization factor (1/\sqrt{|s|}) is selected to keep the energy in the daughter wavelets equal to the energy in the mother wavelet. In other embodiments of wavelet methods, the normalization factor may be chosen differently.

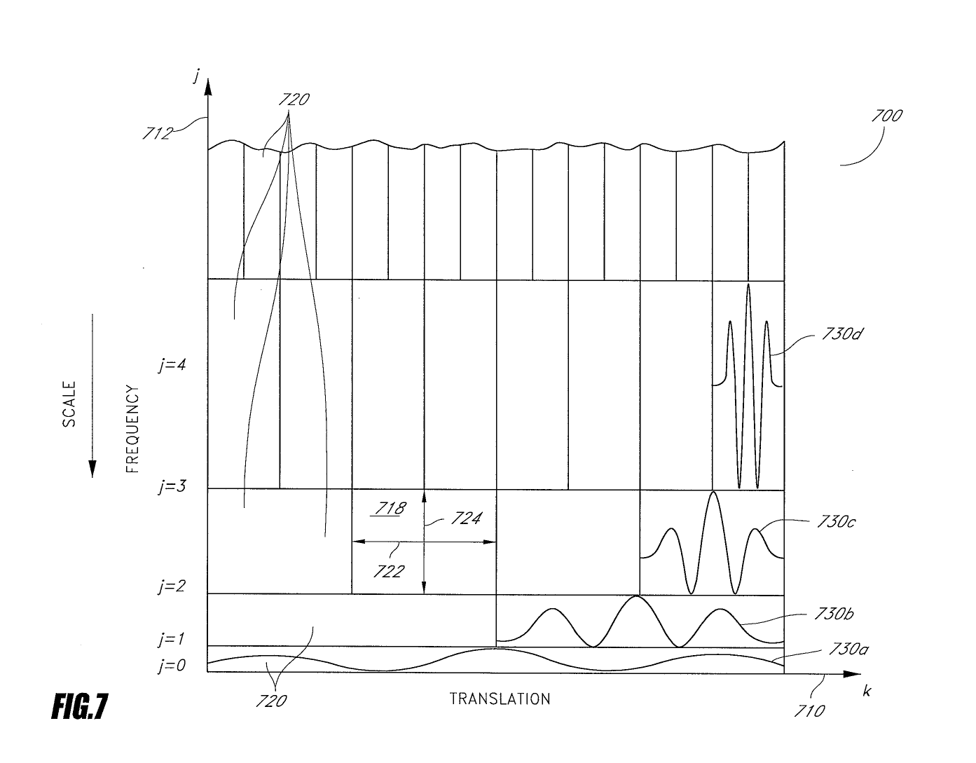

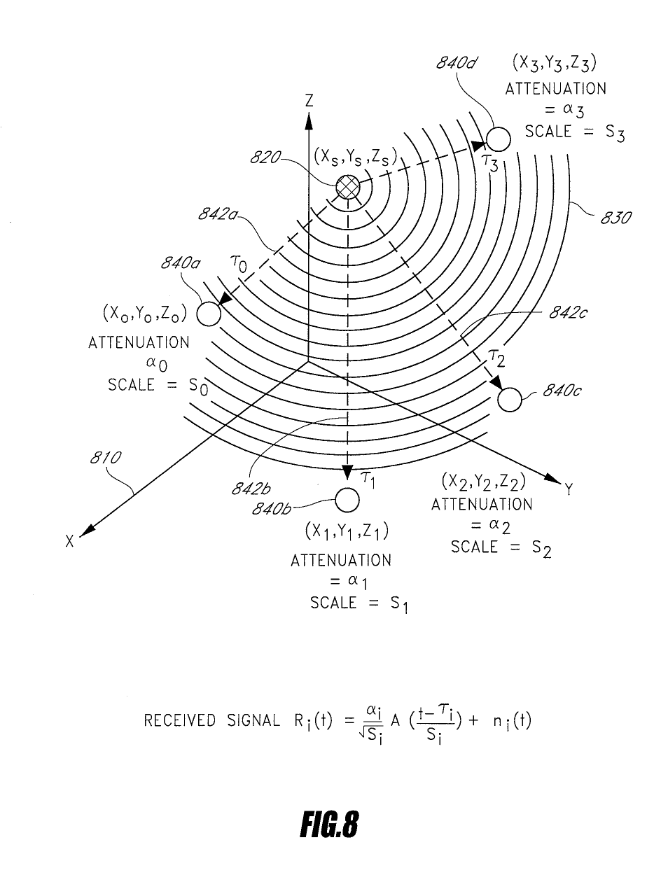

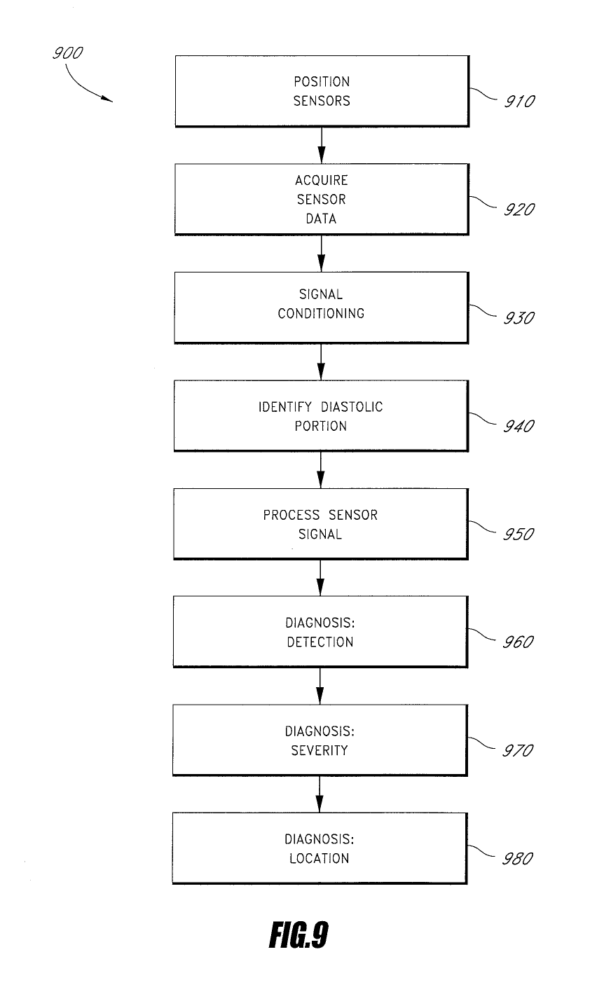

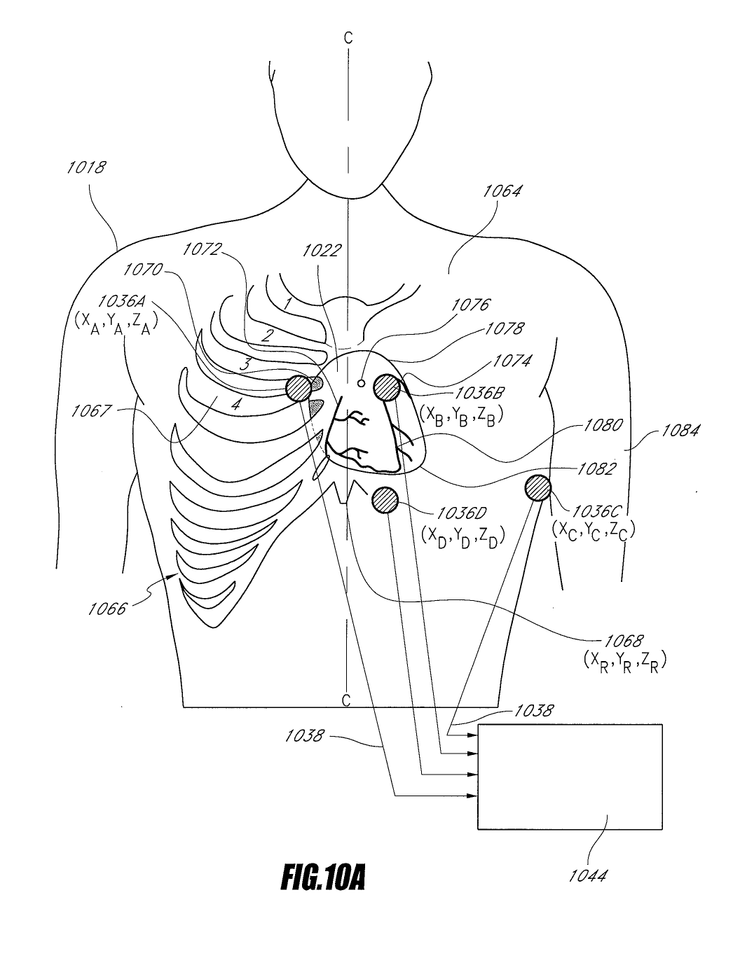

At large values of the scale parameter (low frequencies), the daughter wavelet is a dilated and attenuated replica of the mother wavelet. At small values of the scale parameter (high frequencies), the daughter wavelet is a compressed and amplified replica of the mother. FIGS. 6A-6D illustrate four examples of daughter wavelets 610-640 derived from the Morlet mother wavelet 500. In FIG. 6A, the daughter wavelet 610 is unscaled (e.g., s=1) but is translated (shifted to the right) by τ=5. In FIG. 6B, the daughter wavelet 620 is untranslated (e.g., τ=0) but is scaled by s=5, which results in a dilated and attenuated replica of the mother wavelet 500. In FIG. 6C, the daughter wavelet 630 is untranslated but is scaled by s=½, which results in a compressed and amplified replica of the mother wavelet 500. Finally, in FIG. 6D, the daughter wavelet 640 is both translated (τ=3) and scaled (s=⅓). Although FIGS. 6A-6D illustrate four example wavelets, there are an infinite number of daughter wavelets corresponding to all possible values of the scale and translation parameters. The example wavelets shown in FIGS. 6A-6D illustrate the mathematical ability of wavelets to be arbitrarily shifted and scaled so as to match oscillatory features that may be present in a signal.

An advantage of wavelet analysis is that many mathematical functions can be selected to be the mother wavelet. The mathematical requirements for a function g(t) to be “admissible” (e.g., mathematically allowed) as a mother wavelet are that the function oscillate, have finite energy, and have an average value of zero. A sufficient condition for the function g(t) to be admissible as the mother wavelet is that the following “admissibility constant” c_{g} be finite (less than infinity):

$${c}_{g} ≡ {\int }_{- ∞}^{+ ∞} \frac{{\vert G ( ω ) \vert}^{2}}{\vert ω \vert} ⅆ ω < ∞ \qquad ( 3 )$$

In Eq. (3), G(ω) is the Fourier transform of g(t) and ω is a frequency variable conjugate to t. For the integral in Eq. (3) to be finite, the function G(ω) must equal zero at ω=0, from which it may be shown that the average value of the mother wavelet must be zero:

$${\int }_{- ∞}^{+ ∞} g ( t ) ⅆ t = 0 \qquad ( 4 )$$

Many natural signals satisfy the admissibility conditions and may be used as mother wavelets.

A continuous wavelet transform (CWT) of a signal r(t) with respect to a mother wavelet g(t) is determined from the following integral over all time values:

$$WT [ r , g ] ( s , τ ) = \frac{1}{\sqrt{\vert s \vert}} {\int }_{- ∞}^{+ ∞} r ( t ) g * ( \frac{t - τ}{s} ) ⅆ t . \qquad ( 5 )$$

The asterisk on g denotes complex conjugation. The notation in Eq. (5) indicates that a wavelet transform, WT, is defined by the two quantities inside the square brackets, namely, the input signal r and the mother wavelet g. For any given signal and mother wavelet, the wavelet transform is a function of the two independent scale and translation variables s and τ inside the parentheses.

From the definition of the daughter wavelet in Eq. (2), it is seen that the wavelet transform at any value of scale and translation is an integral of the signal multiplied by the complex conjugate of the daughter wavelet:

$$WT [ r , g ] ( s , τ ) = {\int }_{- ∞}^{+ ∞} r ( t ) {g}_{s , τ}^{*} ( t ) ⅆ t \qquad ( 6 )$$

Accordingly, a value of the continuous wavelet transform is a measure of the match or “overlap” between the signal and the daughter wavelet. The greater the match between the signal and the daughter wavelet, the greater the value of the wavelet transform. One advantage of the continuous wavelet transform comes from the ability of a mother wavelet to be arbitrarily translated and scaled, and then correlated with the signal. Another advantage comes from the ability to preselect a mother wavelet that generally matches the anticipated or known shape of a feature of interest within the signal. In effect, the wavelet transform will perform as a better mathematical microscope to the degree that the mother wavelet better matches the signal feature.

A continuous wavelet transform of a signal contains an enormous amount of information and may be computationally difficult to evaluate at all possible scales and translations. It is common to sample the continuous wavelet transform at a finite number of points in the two-dimensional (s,τ) plane. The sampled transform is known as a discrete wavelet transform (DWT), and a value of the discrete wavelet transform at any one of the finite number of points is known as a wavelet coefficient.

In some embodiments, the finite number of sample points are selected to form a grid (or mesh) in the (s,τ) plane. In some embodiments, the grid of sample points is selected to be a dyadic (base 2) grid, which results in a logarithmic sampling of both the scale and translation parameters. In one embodiment of a dyadic grid, the sample points may be chosen according to:
s=2^{-j} j=0, 1, 2, . . . , J
τ=2^{-j}kτ₀ k=0, 1, 2, . . . , 2^{j} (7)

A pair of integer indices j and k labels the sample points on the grid. The index j corresponds to discrete steps in scale, and the index k corresponds to discrete steps in translation. The scale index j runs from a value of zero to a maximum value of J. Recalling that smaller scale corresponds to larger frequency, Eq. (7) shows that larger values of the index j correspond to higher frequencies. For example, the smallest value of j (j=0) corresponds to the largest scale (s=1) and the lowest frequency of interest in the signal. A larger value corresponds to smaller scales and higher frequencies. As an example, a value of j equal to 10 corresponds to temporal scales that are smaller than the largest scale by a factor of ½¹⁰6= 1/1024 and to frequencies that are correspondingly higher than the lowest frequency of interest by 2¹⁰=1024. Thus, the maximum index value J may be chosen so that the scale parameters in the grid span the entire frequency range of interest. In some embodiments of the apparatus and methods discussed herein, the maximum index value J was equal to 16.

The discrete step in translation is proportional to a time parameter, τ₀, which may be adjusted to suit the problem. For example, in certain embodiments, the time parameter may be set equal to the duration of a heartbeat of a patient or to the duration of the signal received by the sensors. The discrete step in translation also depends on the scale parameter due to the presence of the factor 2^{-j} in Eq. (7). Accordingly, the discrete translation steps are smaller at higher frequencies (higher j). Because the size of the discrete translation step varies with j, the maximum number of translation steps, 2^{j}, also depends on the index j. The size of the discrete time translation step is smaller at higher frequencies so that the wavelets can adequately resolve signal features at those frequencies.

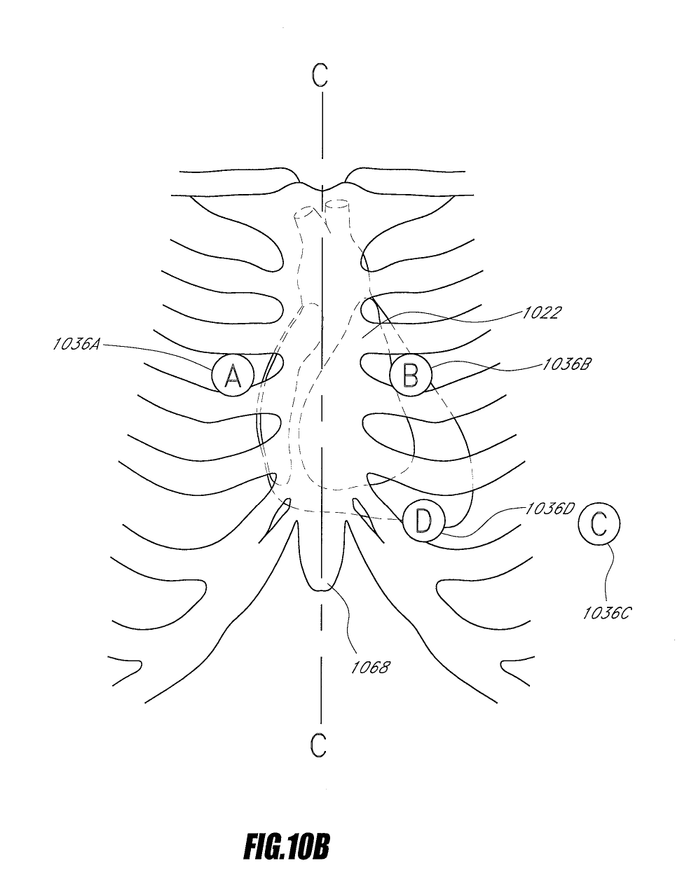

FIG. 7 is a schematic diagram illustrating a plot 700 of the time-frequency resolution of an embodiment of the dyadic grid given by Eq. (7) as a function of the scale and translation parameters. A horizontal axis 710 represents the time translation parameter with τ (and k) increasing toward the right. A vertical axis 712 represents the scale parameter and the frequency, which are inversely related to each other. In FIG. 7, frequency increases upward along the vertical axis 712, therefore, scale increases downward along the axis 712. Smaller values of the index j are at the bottom of the axis 712, while larger values of j are at the top.

FIG. 7 depicts time-frequency resolution cells 720 (the rectangular boxes 720 in FIG. 7) throughout the (s,τ) plane. Resolution refers to the amount of detail in the signal that can be distinguished by the wavelets at any particular sample point. An area 718 of any of the cells 720 corresponds to the wavelet resolution at that location in the plot 700. A horizontal width 722 of the cell 720 represents the resolution in time, whereas a vertical height 724 of the cell 720 corresponds to the resolution in scale (or frequency). The area of a cell 720 is the product of the horizontal width 722 and the vertical height 724.

FIG. 7 shows that frequencies are sampled logarithmically on the dyadic grid. The bottom of the plot 700 represents the lowest frequency. The next higher frequency is double the previous frequency and so forth. Accordingly, the vertical height 724 of the resolution cells 720 increases upward. The choice of the dyadic grid in Eq. (7) results in the sampling frequencies being arranged as octaves. FIG. 7 also shows that the horizontal width 722 of a translation step depends on the scale. For example, the horizontal width 722 of a translation step is smaller at higher frequencies in order that the wavelet transform be capable of resolving higher frequency features in the signal. Since such a small translation step is not needed to resolve more slowly varying features in the signal, the horizontal width 722 of a translation step increases toward the bottom of the plot 700. Accordingly, an advantage of a dyadic grid [e.g., Eq. (7)] is that the translation step automatically adjusts in magnitude to resolve signal features at each frequency.

As shown in FIG. 7, the area 718 of a time-frequency resolution cell 720 depends on the location of the cell in the (s,τ) plane. The area 718 of the cell 720 is in proportion to the “size” of the daughter wavelet used to analyze the signal. FIG. 7 schematically illustrates this proportionality by showing examples of daughter wavelets used at each scale. At lower frequencies, the daughter wavelet 730a is dilated and attenuated in order to correlate with signal features having low frequencies. At higher frequencies, the daughter wavelets 730b, 730c, and 730d are compressed and amplified in order to correlate with signal features having higher frequencies. The mathematical relationship of daughter wavelet to mother wavelet [Eq. (2)] is selected so that the daughter wavelet may effectively resolve signal features at all scales in the (s,τ) plane. In addition, sampling the (s,τ) plane according to a dyadic grid [e.g., Eq. (7)] is advantageous, because the fractional bandwidth (ratio of bandwidth to central frequency) of each time-frequency cell 720 is independent of scale.

In other embodiments, different sample grids may be used. For example, some embodiments may utilize a grid that is linear, rather than logarithmic, in both scale and translation parameters. Other embodiments may utilize a log-linear or linear-log sample grid. Many choices are possible. For example, in certain embodiments, a sample grid similar to Eq. (7) is used, in which the scale index includes the values j=1, 2, 4, 8, 12, and 16, which correspond to frequencies 62.5 Hz, 125 Hz, 250 Hz, 500 Hz, 750 Hz, and 1000 Hz.

As discussed, the continuous wavelet transform defined in Eq. (5) may be evaluated at the sample points of the grid [e.g., Eq. (7)] to form the discrete wavelet transform. The values of Eq. (5) at the sample points are called wavelet coefficients. The wavelet coefficients are an array of numbers that may be labeled by the indices j and k corresponding to the sample grid. In certain embodiments, the signal is represented by a sequence of real numbers. In certain such embodiments, the wavelets are also represented by real numbers [e.g., Eq. (1) is a real-valued function]. Accordingly, in these embodiments, the wavelet coefficients are also real numbers. However, in other embodiments, the signal, the wavelets, or both, may be represented by complex numbers (e.g., numbers having a real part and an imaginary part), and the wavelet coefficients may be complex numbers.

The wavelet coefficients may be evaluated using any of a variety of numerical methods. In one embodiment, the integral in Eq. (5) may be calculated by numerical quadrature techniques, such as, for example, Simpson's rule. In another embodiment, the signal r(t) and the mother wavelet g(t) may be sampled and digitized. At the largest scale (s=1, j=0), the wavelet coefficients are equal to a cross-correlation between the signal and mother wavelet. The integral in Eq. (5) may be calculated by appropriately summing the product of the digitized signal and the mother wavelet. In certain embodiments, machine language multiply-and-accumulate instructions may be used to provide for increases processing speed. Since each subsequent scale is smaller by a factor of two on the dyadic grid, the daughter wavelet at each subsequent scale is a decimated (e.g., subsampled by 2) replica of the mother. Thus, for each value of the scale index j, the wavelet coefficients may be calculated by a decimation-and-summation process, as is well known in the numerical arts. In yet another embodiment, the discrete wavelet transform may be calculated according to a sub-band coding algorithm that involves low-pass and high-pass filtering of the signal.

An advantage of wavelet transform methods is the wide range of choices for the mother wavelet. As discussed above, admissible wavelets may be any function that is oscillatory, has finite energy, and zero average value. Many functions have these properties and can serve as the mother wavelet. Commonly used wavelets have been named after their creators such as, for example, the Morlet, or Daubechies wavelets. Various embodiments of the systems and methods disclosed herein utilize the Haar, Morlet, or Daubechies wavelets. Other embodiments may utilize any other type of continuous or discrete wavelet. For example, some embodiments may use a Hermitian wavelet, a Mexican hat wavelet, a coiflet, a symlet wavelet, or any member of a class of orthogonal or biorthogonal wavelets.

In certain embodiments for diagnosing coronary artery disease, the Morlet wavelet has been found to provide suitable results. In certain such embodiments, the value of the adjustable parameter σ [see Eq. (1)] may be set to correspond to a frequency such as, for example, 10 Hz, 62.5 Hz, 750 Hz, 1000 Hz, or 2000 Hz. In other embodiments, the adjustable parameter σ may be set equal to a frequency corresponding to one of the scales on the sample grid such as, for example, the lowest (or the highest) scale parameter. In other embodiments, mother wavelets other than the Morlet wavelet may be used.

Some embodiments of the systems and methods disclosed herein may utilize a portion of a signal from a diseased heart to construct the mother wavelet. The portion may comprise a signal or a feature of a signal that is characteristic or representative of coronary artery disease. A particular heart signal may be chosen to serve as the mother wavelet, because it most clearly shows the effects of coronary artery disease. The portion of the signal may be used as a template for the construction of the mother wavelet by ensuring that the portion meets the wavelet admissibility conditions. In one embodiment, a representative heart signal may be sampled and digitized before being used as the mother wavelet.

Some embodiments of the systems and methods disclosed herein may use a portion of an individual patient's own heart signal as the mother wavelet. In other embodiments of the systems and methods, the wavelet analysis may be performed with more than one mother wavelet in order to achieve a more accurate diagnosis. In one embodiment, the type of mother wavelet may be modified by a health care professional who administers the methods.

Although certain preferred embodiments may apply the wavelet transform methods discussed herein to the diagnosis of coronary artery disease, it is appreciated that these methods may be applied to the diagnosis or characterization of other diseases, conditions, symptoms, disorders, syndromes, or pathologies. The methods and systems may be applied to other body organs or tissues. Although the methods and systems disclosed herein have been described with reference to human diseases, this is not a limitation, and the methods and systems may be applied to nonhuman animal diseases as well.

Furthermore, some embodiments may utilize wavelet methods in conjunction with Fourier or other signal processing methods to provide additional diagnostic information. For example, in one embodiment, Fourier methods may be used advantageously to determine the frequency range of the turbulent acoustic energy generated by a coronary artery occlusion, while wavelet methods may be used advantageously to determine the severity and/or location of the occlusion.

Detection of Acoustic Energy by Acoustic Sensors

FIG. 8 schematically illustrates further aspects relating to the detection and location of a stenosis 820 that may be present in the body of a patient. It is convenient to identify the location of any point in, on, or surrounding the body by reference to a three-dimensional coordinate system 810. The coordinate system 810 shown in FIG. 8 is a Cartesian coordinate system in which each point is referenced by (x,y,z) coordinates. In other embodiments, different coordinate systems may be used such as, for example, a spherical coordinate system. FIG. 8 shows the stenosis 820 located at a coordinate position (xₛ,yₛ,zₛ). The stenosis 820 generates acoustic energy 830 that is transmitted into the patient's body. One or more acoustic sensors 840a-840d are positioned at locations (xᵢ,yᵢ,zᵢ) where an integer i is an index that labels each sensor. If the total number of sensors is denoted by N, the index i ranges from i=0 (for the first sensor) to i=N−1 (for the N^{th} sensor). For example, FIG. 8 shows the first sensor 840a located at (x₀,y₀,z₀), the second sensor 840b located at (x₁,y₁,z₁), the third sensor 840c located at (x₂,y₂,z₂), and the fourth sensor 840d located at (x₃,y₃,z₃). The total number of sensors may be one, two, three, four, five, or more. In the embodiment shown in FIG. 8, four sensors are illustrated, although fewer or more sensors may be used in other embodiments.

The acoustic energy 830 propagates along paths 842a-842d from the stenosis 820 to each sensor 840a-840d. The length d of the path from the stenosis at (xₛ,yₛ,zₛ) to the i^{th} sensor at (xᵢ,yᵢ,zᵢ) is determined from Pythagoras's equation
dᵢ²=(xᵢ−xₛ)²+(yᵢ−yₛ)²+(zᵢ−zₛ)². (8)

In certain non-invasive embodiments, the sensors 840a-840d are positioned on the surface of the patient's body. However, in other embodiments, one or more sensors 840a-840d may be located within the body cavity of the patient. It is preferable, although not necessary, that the sensors 840a-840d be located at positions that substantially surround the stenosis 820 and at positions that are not in a substantially colinear or a substantially coplanar configuration. For example, the sensors 840a-840d may be aligned so that they provide a three-dimensional view of the heart from a wide range of viewing angles. In some embodiments, the sensors 840a-840d are placed at heart auscultation points. In some embodiments, it is preferable, although not required, that the sensors 840a-840d be placed at positions such that the acoustic energy 830 from the stenosis 820 to each sensor 840a-840d travels through substantially similar types of body tissue. In these embodiments, the value of the sound speed is substantially the same along each of the paths 842a-842d. For example, it is advantageous to select a path that avoids a substantial portion of lung tissue, because the speed of sound in air (about 340 m/s) is substantially different from the speed of sound in soft body tissue (about 1540 m/s). Similarly, in some advantageous embodiments, paths comprising substantial portions of bone (sound speed equal to about 4000 m/s) are avoided.

Wavelet Transform Methods Applied to Acoustic Signals

The following example model illustrates one embodiment of wavelet transform methods for diagnosing the presence and/or location of the coronary artery stenosis in an acoustic signal. This example model is not intended to be a limitation to the scope of the disclosed systems and methods but rather is intended to be an illustrative example of wavelet transform methods. In other embodiments of the apparatus and methods, different models for the acoustic signal and its propagation through body tissue can be adopted.

In the example model, the stenosis is assumed to emit an acoustic signal A(t). The acoustic signal includes sound waves generated by the turbulent flow of blood past the stenosis. The turbulent flow may generate sound waves having frequencies in the range of about 500 Hz to about 1000 Hz. As shown in FIG. 8, the acoustic energy 830 propagates throughout the body and along the paths 842a-842d to each of the sensors 840a-840d where the signal is received. In the example model, the propagation speed is assumed to be a constant value c, which may be taken to be 1540 m/s, which is the acoustic propagation speed in soft body tissue. However, in other models, a non-constant value could be used.

As the acoustic signal propagates along the path to the i^{th} sensor, the signal is attenuated and dilated (scaled) by absorption and scattering from body tissue. Additionally, the signal requires a finite propagation time τᵢ to reach the i^{th} sensor due to the finite speed of sound. The propagation time τᵢ is related to the length of the path and the speed of sound by the constant velocity kinematic equation τᵢ=dᵢ/c. Lastly, the signal may be degraded by noise. The combination of these physical effects suggests that the acoustic signal received by the i^{th} sensor may be modeled as

$${R}_{i} ( t ) = \frac{{α}_{i}}{\sqrt{{s}_{i}}} A ( \frac{t - τ}{{s}_{i}} ) + {n}_{i} ( t ) . \qquad ( 9 )$$

In Eq. (9), αᵢ represents the attenuation, sᵢ represents the dilation (scaling), and τᵢ represents the propagation time of the acoustic signal between emission from the stenosis 820 and reception at the sensors 840a-840d. The noise in the signal, nᵢ(t), is assumed to be a random statistical process that is uncorrelated with the emitted signal A(t).

A wavelet transform of the signal received at the i^{th} sensor can be taken by substituting Rᵢ(t) into Eq. (5). The wavelet transform of the noise term nᵢ(t) averages to zero, because the noise is uncorrelated with the mother wavelet g(t). The resulting wavelet transform of the received signal at the i^{th} sensor can be written as

$$WT [ {R}_{i} , g ] ( s , τ ) = {α}_{i} WT [ A , g ] ( \frac{s}{{s}_{i}} , \frac{τ - {τ}_{i}}{{s}_{i}} ) . \qquad ( 10 )$$

Equation (10) shows that the wavelet transform of the received signal, which is a readily measurable quantity, is equal to the attenuation parameter αᵢ multiplied by the wavelet transform of the acoustic signal A(t) emitted by the stenosis 820. Accordingly, even though the emitted signal A(t) is modified by the physical effects of attenuation, dilation, translation in time, and degradation by noise, the underlying properties of the emitted signal nonetheless may be inferred from the wavelet transform in Eq. (10). However, Eq. (10) shows that these physical effects require the wavelet transform of the emitted signal to be evaluated at the scaled and shifted arguments shown in the parentheses on the right-hand-side.

In certain embodiments of the systems and methods disclosed herein, the mother wavelet g(t) is selected to have a shape that generally matches signal features that are characteristic or representative of coronary heart disease. As an illustration, the mother wavelet g in the example model will be assumed to be directly proportional to the signal feature A. In this example illustration, the wavelet transform on the right hand side of Eq. (10) is expected to have a local maximum value when the mother wavelet is unshifted and unscaled, e.g., at (1,0). By setting the arguments in the rightmost parentheses in Eq. (10) to (1,0), it is seen that the measured wavelet transform WT[Rᵢ,g] has a local maximum value at s=sᵢ and τ=τᵢ. Thus, by identifying the peaks in the measured wavelet transform WT[Rᵢ,g], the values of the dilation parameter sᵢ and the propagation time τᵢ can be estimated for each of the sensors. The propagation times τᵢ derived from the wavelet transforms may be used in systems and methods that calculate the location of the stenosis 820 as further discussed below.

The peaks in the wavelet transform coefficients may be identified by many well known numerical techniques. In some embodiments, for example, the peaks of the absolute magnitude of the wavelet coefficients are identified, while in other embodiments, the peaks in the squared value of the wavelet coefficients, which are more representative of the acoustic energy in the signal, are identified. The presence of more than one peak in the data may indicate the presence of more than one stenosis in the patient's coronary arteries.

The example model discussed above is intended to provide an illustration of the results available from the wavelet analysis of acoustic signals received from body organs or tissues. The example model is not intended to limit the scope of wavelet transform techniques that are in accordance with the principles disclosed herein. Equations and results analogous to these may be developed for different mathematical models that incorporate different assumptions. For example, in certain embodiments, one of the received signals is treated as a reference signal, and the mother wavelet is selected from a portion of this reference signal. The portion may be appropriately scaled and shifted to ensure that the wavelet admissibility criteria are satisfied. In certain such embodiments, each sensor in turn may be treated as the reference signal so as to generate a larger set of data, which may increase accuracy and precision. In some preferred embodiments, peaks of the wavelet transforms of the received signals can be used to calculate the acoustic signal dilation parameters and propagation times. Further details of methods related to these and other embodiments may be found in U.S. Pat. No. 6,178,386 entitled “Method and Apparatus for Fault Detection,” issued on Jan. 23, 2001, which is hereby incorporated by reference herein in its entirety and made a part of this specification.

Accordingly, in certain embodiments, each received acoustic energy signal is processed by a wavelet transformation, and the peaks of the wavelet transform may be used to determine the dilation and propagation time parameters. In other embodiments, dilation and propagation time parameters may be determined using further mathematical or statistical analysis methods such as, for example, mean and variance analyses.

Determining Stenosis Coordinate Location

In some embodiments, the wavelet transform methods discussed above are used to determine the times τᵢ it takes the acoustic energy 830 to propagate from the stenosis 820 to each of the sensors 840a-840d (see FIG. 8). It is known that the position of the stenosis 820 may be inferred from the propagation times under certain conditions. A variety of methods may be used to calculate the coordinate location (xₛ,yₛ,zₛ) of the stenosis 820 using data from the wavelet transform methods discussed above.

Certain embodiments use a time difference of arrival (TDOA) method to determine the location of the stenosis 820. In these embodiments, one of the sensors is defined to be a reference sensor (e.g., the first sensor indexed by i=0), and a TDOA measures the difference in propagation times to one of the other N−1 sensors relative to the reference sensor. Thus, the TDOA for the i^{th} sensor is determined from
Δτᵢ=τᵢ−τ₀, (i=1, . . . , N−1). (11)
The range difference (RD) corresponding to the TDOA is the difference between the length of the acoustic propagation path from the stenosis 820 to the i^{th} sensor (dᵢ) and the length from the stenosis 820 to the reference sensor (d₀). Assuming the speed of sound c is the same for all propagation paths 842a-842d, the range differences may be related to the time differences of arrival by using constant velocity kinematics and Pythagoras equation Eq. (8):

$$\begin{matrix}c Δ {τ}_{i} = {d}_{i} - {d}_{0} ( i = 1 , … , N - 1 ) \\ = \sqrt{{( {x}_{s} - {x}_{i} )}^{2} + {( {y}_{s} - {y}_{i} )}^{2} + {( {z}_{s} - {z}_{i} )}^{2}} - \\ \sqrt{{( {x}_{s} - {x}_{0} )}^{2} + {( {y}_{s} - {y}_{0} )}^{2} + {( {z}_{s} - {z}_{0} )}^{2}}\end{matrix} \qquad ( 12 )$$

Since the sound speed c is assumed to be a known value, and the sensor coordinates and the TDOA's are measured quantities, Eq. (12) represents N−1 equations for the three unknown coordinates of the stenosis. Accordingly, the number of sensors N must be greater than or equal to four to find a unique solution for the location of the stenosis 820. Certain preferred embodiments adopt a value for the sound speed that is representative of soft body tissue (1540 m/s).

In some embodiments, a centroid algorithm is used to find the TDOA's in Eq. (11). An arrival time is determined as the centroid of a portion of the wavelet transform of an acoustic signal, and the TDOA is the difference between the arrival times for two sensors, e.g., the i^{th} sensor and the reference sensor. The centroid algorithm uses a weighted sum to determine the arrival time. In certain embodiments, the portion of the wavelet transform used in the centroid algorithm corresponds to the diastolic portion of a heartbeat. In some of these embodiments, the portion corresponds to the wavelet transform at a preselected value of the scale parameter, for example, a scale corresponding to a characteristic turbulent frequency. In certain such embodiments, the scale j=12, which corresponds to acoustic sounds at 750 Hz, is selected. In certain embodiments, the centroid algorithm uses as weight coefficients the absolute values of the wavelet coefficients, |WT(j,k)|, for a preselected value of the scale (e.g., j=12) and for all translations (k) falling within the diastolic portion of the signal. In other embodiments, the square of the wavelet coefficients, which is representative of acoustic energy, may be used as weight coefficients, or a different weighting function may be chosen. Many variations are possible.

In some embodiments, a maximum value of the wavelet array for the acoustic signal received by the reference sensor (e.g., i=0) is determined, and the value of the translation parameter corresponding to the maximum value is stored as a peak index k₀. The centroid of the signal is evaluated over a portion of the wavelet array corresponding to a window of length L on either side of the peak index k₀. The value of L may depend on the sampling rate of the signals. In an embodiment in which the sampling rate is 22 kHz, the window length is equal to five sample periods. The centroid of the i^{th} signal is denoted by Cᵢ and is defined as:

$${C}_{i} = \frac{{\sum }_{k = {k}_{0} - L}^{{k}_{0} + L} k \vert {WT}_{i} ( j , k ) \vert}{{\sum }_{k = {k}_{0} - L}^{{k}_{0} + L} \vert {WT}_{i} ( j , k ) \vert} , \qquad ( 13 )$$

where WTᵢ is the wavelet transform of the i^{th} signal. Eq. (13) is used to determine the centroid of the reference signal, C₀, and the centroid of the signals received by each of the other sensors, Cᵢ. The TDOA [see Eq. (11)] for the i^{th} sensor is defined as an arithmetic difference between these values: Cᵢ−C₀. The use of centroid values, rather than peak index locations, may improve the accuracy by which TDOA's can be evaluated.

In other embodiments of the centroid algorithm, rather than using a preselected scale (such as j=12), the weighting coefficients used in Eq. (13) correspond to an average of the wavelet coefficients. In one such embodiment, the wavelet coefficients are averaged over scale parameters that correspond to the turbulent acoustic signal. In other embodiments, a square of the wavelet coefficients is used in Eq. (13).

The presence of more than one peak in the wavelet coefficient data may indicate the presence of more than one stenosis in the patient. Accordingly, in some embodiments, multiple peaks are used to identify the coordinate location of multiple stenoses.

The accuracy by which the coordinate location of a stenosis may be determined will depend on the sampling frequency, because the sampling frequency limits the accuracy by which TDOA's may be measured. At higher sampling frequencies, the coordinate location of the stenosis may be determined to higher accuracy than at lower sampling frequencies. In some embodiments of the present diagnostic apparatus, a sampling frequency of 22 kHz is used, and the coordinate location of the stenosis may be determined to lie within the patient's chest cavity. In other embodiments of the apparatus, a sampling frequency higher than 22 kHz may be used such as, for example, 120 kHz. In embodiments utilizing a higher frequency, the coordinate location of the stenosis may be determined within an accuracy corresponding to, for example, one quadrant of the heart. At sufficiently high sampling frequencies, the coordinate location of the stenosis may be determined within 1 cm or less.

To relate the coordinate location of the stenosis to a physical position within the patient's heart (e.g., to a position within a particular coronary artery), the orientation of the heart within the chest cavity is needed. In some embodiments, the orientation and location and other anatomical structures of the heart may be determined by additional medical procedures such as, for example, an electrocardiogram, an ultrasound, CAT scan, magnetic resonance image (MRI), or X-ray image. Such information can be transferred electronically to an embodiment of an acoustic sensing and processing device. Such information can be input into an acoustic processing device by means of an input device such as, for example, a keypad, touchscreen, voice recognition, etc. In other embodiments, the heart orientation may be estimated by an examination performed by a health care professional. Other clinical procedures may be used in other embodiments.

In some embodiments, having determined a set of TDOA's (e.g., by any of the aforementioned methods) and having an estimate of the sound speed c (e.g., 1540 m/s), the coordinate location of the stenosis can be determined from Eq. (12). In some embodiments, Eq. (12) is solved by an iterative least squares method to find a “best fit” stenosis location. In other embodiments, statistical methods such as, for example, a maximum likelihood algorithm, are used to determine a most probable solution to Eq. (12). In certain preferred embodiments, a closed-form solution to Eq. (12) is used to directly determine the stenosis location, because closed form solutions generate accurate locations and are less computationally demanding than iterative, nonlinear, or statistical methods. Some embodiments utilize the closed-form algorithm described by Mellen, et al., “Closed-Form Solution for Determining Emitter Location Using Time Difference of Arrival Measurements,” IEEE Transactions on Aerospace and Electronic Systems, pp. 1056-1058, vol. 39, No. 3, July 2003, which is hereby incorporated by reference herein in its entirety and made a part of this specification. In other embodiments, the sound speed may be treated as an unknown quantity that is to be determined along with the stenosis location.

Methods of Operation of Preferred Embodiments

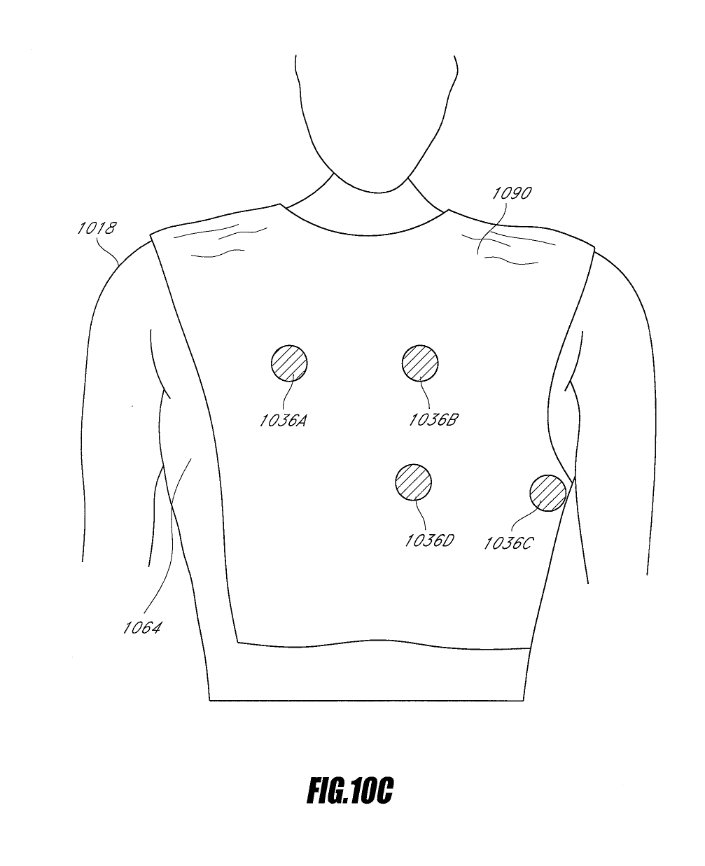

FIG. 9 illustrates a flowchart 900 showing an embodiment of a method for diagnosing the presence, the severity, and/or the location of one or more stenoses in a patient's coronary arteries. Blocks 910-980 represent functions, modules, or operations that may be performed in the embodiment of the method. In other embodiments, additional or different Blocks may be utilized, and the additional or different Blocks may occur in a different order. The flowchart 900 illustrates one embodiment of the methods disclosed herein, however, the flowchart 900 is not intended as a limitation to the scope of the methods but rather is intended as an illustrative flowchart that is in accordance with the principles herein disclosed.

In Block 910, one or more acoustic sensors are positioned on the body of the patient in preparation for the determining the presence, severity, and/or location of stenoses. For the purposes of teaching the details of certain preferred embodiments, the following discussion will assume that four acoustic sensors are utilized. However, this is not a limitation on the methods, and other embodiments may use fewer or more sensors.

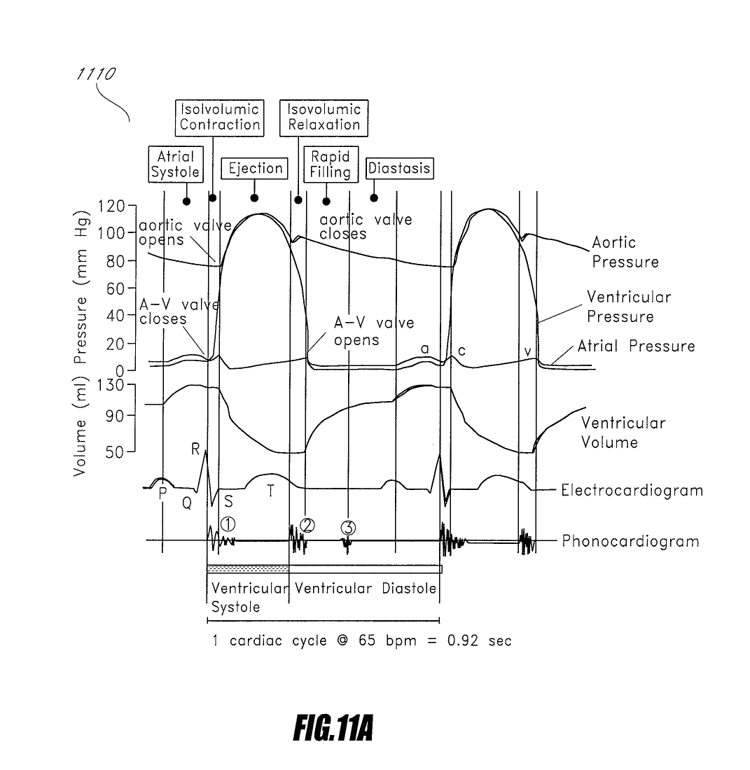

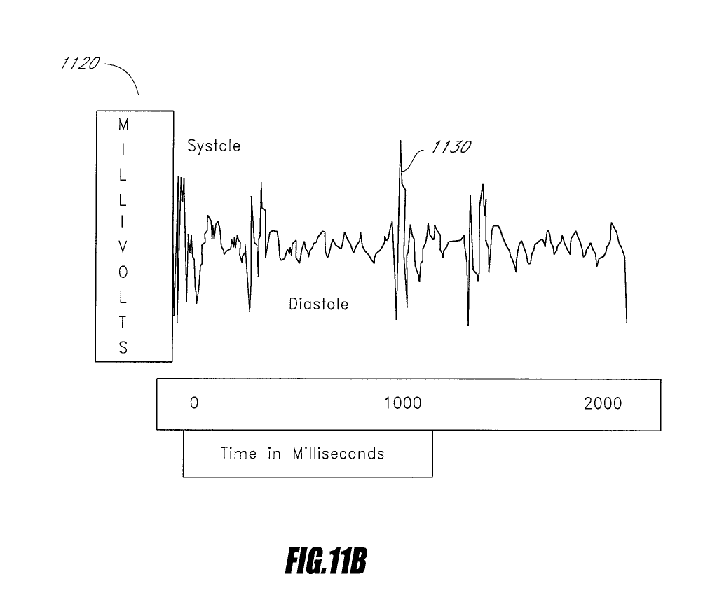

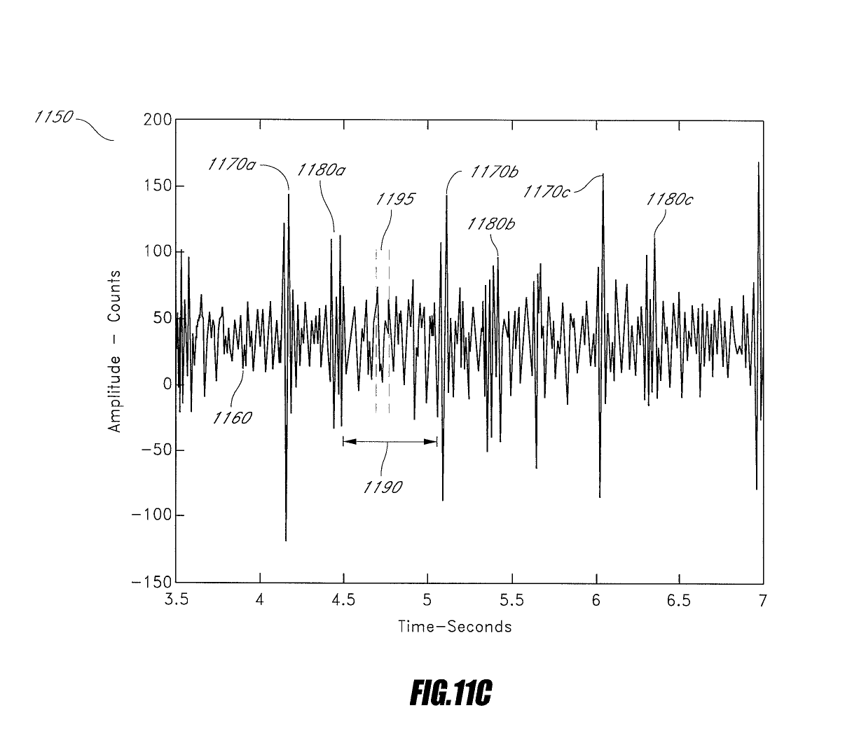

FIG. 10A illustrates one preferred embodiment for positioning the acoustic sensors. In this embodiment, four sensors 1036A-1036D are attached to the chest 1064 of the patient 1018 at known locations with respect to the heart 1022, ribs 1066, a base of the sternum 1068 (the xiphoid process), and a center line C-C. For reference, FIG. 10A shows right and left coronary arteries 1072 and 1074. A left border of the heart 1022 is identified with the reference numeral 1078. A stenosis 1076 is also shown in FIG. 10A.

In some embodiments, the sensors 1036A-1036D are configured to be in electrical communication with an apparatus 1044 that is configured to perform the signal processing analysis discussed herein with reference to Blocks 920-980. In the embodiment shown in FIG. 10A, electrical wires 1038 are used to establish electrical communication between the sensors 1036A-1036D and the apparatus 1044, although in other embodiments electrical communication may be established by methods such as, for example, wireless communication using electromagnetic radiation.

In some embodiments, the base of the sternum 1068 may be used as a reference point R having coordinates (x_{R},y_{R},z_{R}). For convenience, the reference point R may be selected to be the origin of the coordinate system 810 shown in FIG. 8. In the embodiment shown in FIG. 10A, the sensor 1036A is positioned at a point A, having coordinates (x_{A},y_{A},z_{A}); the sensor 1036B is positioned at a point B, having coordinates (x_{B},y_{B},z_{B}); the sensor 1036C is positioned at a point C, having coordinates (x_{C},y_{C},z_{C}); and the sensor 1036D is positioned at a point D, having coordinates (x_{D},y_{D},z_{D}).

In certain embodiments, the sensor 1036A is positioned near a right border 1070 of the heart 1022. For example, the sensor 1036A may be located on the right side of the chest 1064 just above the fourth rib 1067 and approximately one inch to the left of the center line C-C. In some of these embodiments, the sensor 1036B is aligned opposite to the sensor 1036A and spaced approximately one inch to the right of the center line C-C. In the embodiment shown in FIG. 10A, the sensor 1036B is positioned near the left anterior descending artery 1080. In some embodiments, the sensor 1036C is aligned with the apex 1082 of the heart 1022 on the left side of the patient 1018 between the chest 1064 and the upper arm 1084. The sensor 1036D is located approximately one inch to the right of the center line C-C and is aligned with the base of the sternum 1068.

FIG. 10B illustrates another embodiment for the positioning of the acoustic sensors on the body of the patient. In this embodiment, the sensor 1036A is positioned at the third intercostal space, two inches to the right of the center line C-C passing through the base of the sternum 1068 (the xiphoid process). The sensor 1036B is located at the third intercostal space, two inches to the left of the center line C-C. The sensor 1036B is located at the fifth intercostal space, two inches to the left of the center line C-C. The sensor 1036D is located below the sensor 1036B at the fifth intercostal space, two inches to the left of the centerline C-C. Finally, the sensor 1036C is located on the mid-axillary line, approximately level with the sensor 1036D. All positions described are relative to the patient's point of view.

In some embodiments of the methods disclosed herein, the sensor positions 1036A-1036D may be left to the judgment of a health care professional administering the diagnostic procedure. For example, after performing an auscultation of the patient's chest (e.g., by listening with a stethoscope), the health care professional may position the sensors based on results of the auscultation. In these embodiments, the health care professional's personal knowledge of the patient's anatomy may be used to position the sensors in advantageous locations.

In some embodiments, after positioning the sensors 1036A-1036D, the coordinate locations of the sensors may be determined. FIG. 10C illustrates an embodiment for placing the sensors 1036A-1036D and for determining their respective coordinates. In this embodiment, the sensors 1036A-1036D are attached to a chest template 1090 of known size and shape that is worn by the patient 1018 during testing. Since the chest template 1090 has a known size, the coordinate locations of the sensors 1036A-1036D may be accurately determined The chest template 1090 is positioned so that it substantially surrounds the chest 1064 of the patient 1018 while the acoustic measurements are being taken. In some embodiments, the chest template 1090 may be fabricated from paper or fabric and may comprise holes or markings where the sensors 1036A-1036D are to be located. The chest template 1090 may be fabricated in a variety of sizes and shapes. In certain embodiments, the chest template 1090 may be fabricated from a stretchable material that can conform to the patient's chest. A benefit of the chest template 1090 is that it preserves the modesty of the patient during the examination.

In other embodiments, one of the sensors (e.g., the first sensor 1036A) is located at the reference position R at the base of the sternum 1068. The coordinates of the other sensors (e.g., the sensors 1036B-1036D) are determined by measuring a length and a direction of an arc from the first sensor 1036A to each of the other sensors 1036B-1036D along the surface of the body of the patient 1018. In some embodiments of the method, a health care professional may use a ruler or a tape measure to determine the length of the arc in one or more predetermined directions relative to a reference sensor or to one or more of the other sensors. The health care professional may enter the measurements directly into the diagnostic device (e.g., via a keypad, touchscreen, or pointing device) or may notate the measurements on a patient chart or report or clinical record for subsequent data entry. The length and direction of the arc may be converted to Cartesian coordinates of the sensor by using principles of Euclidean geometry. In one embodiment, the health care professional marks the following distances on the patient chart or report: a distance between sensors 1036A and 1036B measured horizontally across the patient's chest 1064 (e.g., along a line that is substantially perpendicular to the centerline C-C shown in FIGS. 10A, 10B); a distance along centerline C-C from the reference position R at the base of the sternum 1068 to a line connecting sensors 1036A and 1036B; a distance between the reference position R and the sensor 1036D; and, a distance between the sensor 1036D and the sensor 1036C.

In other embodiments, each of the sensors may transmit a signal that is received by one or more of the other sensors. The sensor coordinates may be determined using standard echolocation or triangulation techniques. In still other embodiments, the coordinates are determined by reference to another point, such as a location on a portable device, similar in principle or identical to a satellite navigation system such as, for example, the Global Positioning System (GPS).

In Block 920 of the embodiment of the flowchart 900 shown in FIG. 9, acoustic signals are acquired by the sensors placed on the body of the patient in Block 910. In certain preferred embodiments, the acoustic signals are emitted by the patient's heart and may include signal features that may be used to diagnose the presence and/or severity of coronary artery disease.

The sensors are responsive to the acoustic energy emitted by an organ or other biological entity such as the heart, which includes an acoustic signal from one or more stenoses. The sensors can be shielded from ambient noise and configured to sense acoustic signals emanating from within the body. For example, in some embodiments, sensors are acoustically coupled to the skin of the patient. As further described herein with reference to FIG. 8, each sensor detects the analog acoustic energy and communicates a signal representative of the acoustic energy to additional hardware/software components for signal processing. In some of these embodiments, the sensors transmit an analog signal that is sampled and digitized by other components such as, for example, an analog-to-digital converter (ADC). In other embodiments, the sensors transmit a digitized signal. In certain embodiments, the sensors comprise ultrasound transducers, which may comprise ultrasound transmitters, receivers, microphones, and/or piezoelectric devices. In some embodiments, one or more of the sensors may be used for calibration purposes. Further details of sensors suitable for use with embodiments of Blocks 910 and 920 are discussed below.

In some embodiments, after the sensors are placed on the patient's body, a validation process may be performed before heart measurements are taken. A validation step advantageously increases the likelihood that the sensors are correctly placed on the body and that the signal from each sensor corresponds to a body signal, e.g., a heartbeat, rather than an extraneous signal, such as room noise. Additionally, a validation step decreases the likelihood that data from faulty sensors will be used in the subsequent analysis.

In some embodiments of the validation process, the acoustic signal from each sensor is sampled at a rate such as, for example, 350 Hz. The data from each sensor is checked for clipping, e.g., that the signal's amplitude is between an upper limit (such as 4090 counts) and a lower limit (such as 0 counts). Various statistical parameters are calculated to indicate whether a body signal, an extraneous noise signal, or no signal (flatline) is being acquired by each sensor. In some embodiments, values of a statistical variance and a slope of the signal are updated periodically. For example, the variance and the slope may be recalculated each time a predetermined portion of a signal is received. In one embodiment, the size of the predetermined portion is selected to be the sampling rate multiplied by a sampling time, such as 1/25 second. If, for example, the variance or the slope are outside ranges expected from the signal, the diagnostic apparatus can communicate a fault code to the patient, user, doctor, clinician, diagnostician, or health care professional. The fault code indicates that one (or more) of the sensors may not be detecting a body signal. The health care professional performing the examination may then reposition the sensors and restart the measurement. The fault code may include an auditory signal (e.g., a sound such as a bell or tone). In some embodiments, the diagnostic apparatus will prevent further signal acquisition until the fault code is cleared. In certain preferred embodiments, if the variance of the signal received from a sensor is outside a range from, for example, 10 to 500,000 counts², the fault code is communicated to the health care professional. In other embodiments, the validation process may determine whether to send the fault code based on a comparison of a received signal to an expected signal such as, for example, a reference heartbeat signal.

In yet other embodiments, the validation process may include a self-test procedure in which one or more of the sensors transmits a signal to be received by the other sensors. If the transmitted signal is not received, is distorted, or has a signal-to-noise ratio too low for useful diagnostic measurements, a fault code may be communicated to indicate a potential malfunction.

During the signal acquisition process, it is preferred, but not necessary, that the patient sit in an upright position and hold his or her breath. In certain embodiments, about eight seconds of data is taken, which typically comprises about 6 to 16 heartbeats. In other embodiments, data is taken for whatever length of time the patient can comfortably hold his or her breath. In other embodiments, the patient's breathing sounds are identified and filtered out so that the desired acoustic measurements can be taken during breathing.

In Block 930 of the embodiment of the flowchart 900 shown in FIG. 9, the acoustic signals may be conditioned for example by, amplifying, filtering, synchronizing, digitizing, and/or multiplexing the signals for further processing by additional hardware or software modules or components. The acoustic signal emitted by the stenosis is attenuated by geometrical effects (e.g., the inverse square law) and by absorption and scattering by body tissues. Therefore, in some embodiments, the signal from each of the sensors is amplified, and the amplifier gain may be different for different sensors. In some embodiments, the analog acoustic signals are low-pass filtered to remove unwanted high frequency noise components and to ensure that the subsequent digital signal is Nyquist sampled. The passband of the acoustic filter must be sufficiently high for the signal frequencies of interest to pass without significant attenuation. For example, in some embodiments, the analog signal is low-pass filtered to remove signal frequencies above 2.5 kHz. This choice of low-pass filter preserves the turbulent acoustic energy generated by coronary occlusions, which typically occurs from about 500 Hz to about 1000 Hz. The filtered analog signal may be sampled at rates above 5.0 kHz to ensure that no aliasing effects are present in the resulting digital signal. In one embodiment, a sample rate of 22 kHz is used, which is well above the minimum frequency required by the Nyquist criterion. In other embodiments, the analog acoustic signal may be high pass filtered to remove low frequency components such as, for example, patient breathing and heart valve

After sampling, the digital signal may be passed through one or more digital filters. In certain embodiments, the filter may be a linear filter and may comprise a finite impulse response (FIR) filter and/or an infinite impulse response (IIR). For example, one embodiment uses an FIR low pass filter of order 100, with passband frequency 1100 Hz, stopband frequency 1500 Hz, and passband ripple less than or equal to 0.5 dB. The digital filter advantageously may remove noise and other spurious high frequency components in the signal. Other filters can be used such as, for example, a high pass filter, a band-pass filter, a band-stop filter, a notch filter, or other suitable filter. The filter may include a Wiener filter or a Kalman filter, for example. In certain embodiments, the digital signal is high pass filtered to remove low frequency components due to, for example, patient breathing and heart sounds representative of the basic heart cycle. In certain such embodiments, the high pass filtering is performed after the individual heartbeats are identified (e.g., after Block 940 is performed). In one embodiment, a band-pass filter is used to attenuate frequency components of the signal outside the range from about 300 Hz to 1500 Hz.

In some embodiments, one or more conditioning procedures are performed on the digital signal. The conditioning procedures may include digital filtering as described above. In certain embodiments, the conditioning procedure comprises a transform applied to the digital signal so as to produce information relating to a frequency spectrum for the digital signal. For example, in certain such embodiments, the digital signal is Fourier transformed so as to produce a spectrum indicative of, for example, the acoustic energy (or power) received by the sensors in a range of frequency intervals. The frequency spectrum can be used to identify portions of the digital signal that comprise turbulent acoustic energy emitted from, for example, a coronary artery stenosis. In some embodiments, characteristics of the digital filter may be determined based on the results of the frequency spectrum. For example, the passband of a band-pass filter may be determined by identifying a frequency range in the frequency spectrum that is likely to comprise turbulent acoustic energy at a signal-to-noise level suitable for diagnostic analysis. Additionally, the frequency spectrum may be used to characterize noise in the system in order to suitably filter out the noise, for example, by applying a Wiener filter to the digital signal.

In other embodiments, other conditioning procedures may be applied to the digital signal. For example, a parametric modeling procedure may be used such as, for example, a moving average method, or an autoregressive model, or an autoregressive moving average model. In certain embodiments, the conditioning procedure may include correlation methods such as, autocorrelation or cross-correlation methods, either in the time domain or the frequency domain.

In certain patients, the acoustic signal from a stenosis may have a relatively low amplitude in comparison to the other acoustic signals emanating from the body of the patient. Accordingly, it is advantageous to analyze portions of the acoustic signal acquired when background sound waves are at a reduced amplitude. In Block 940 of the embodiment of the flowchart 900 shown in FIG. 9, a suitable portion of the signals received by the sensors is selected for analysis in Blocks 950-980.

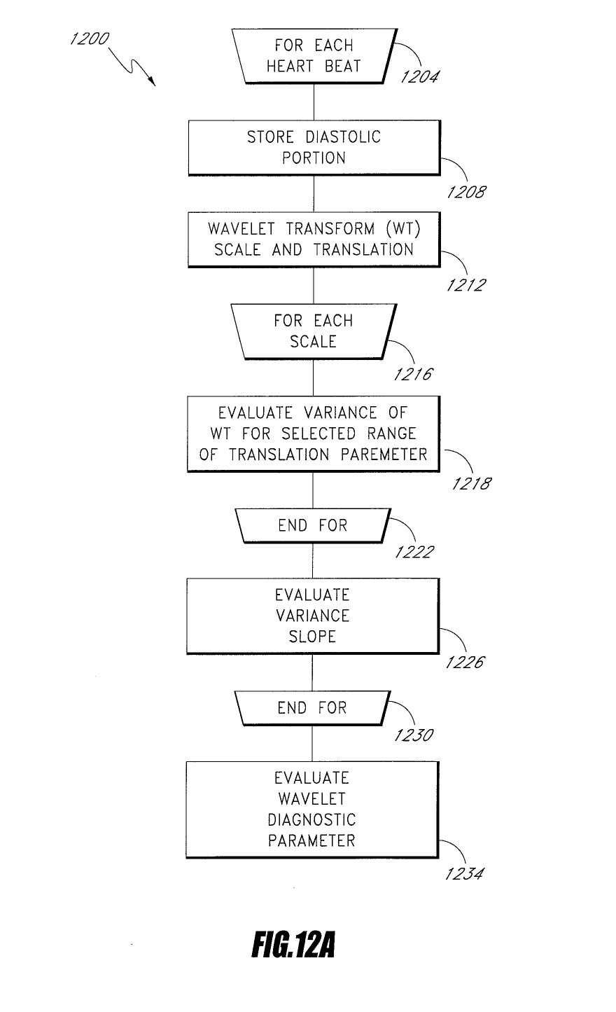

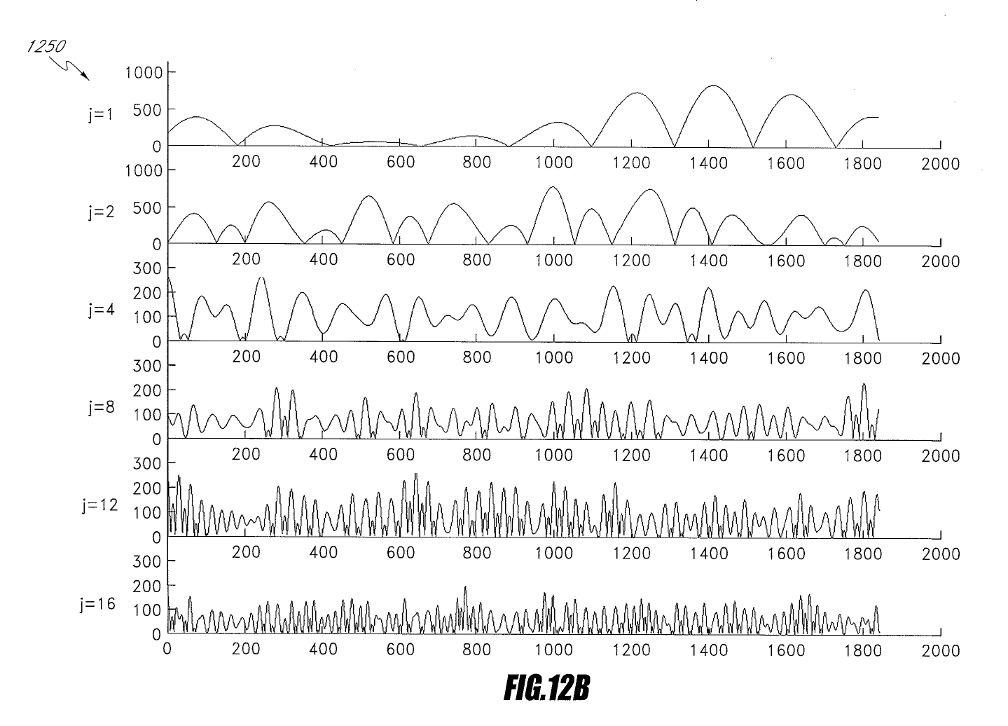

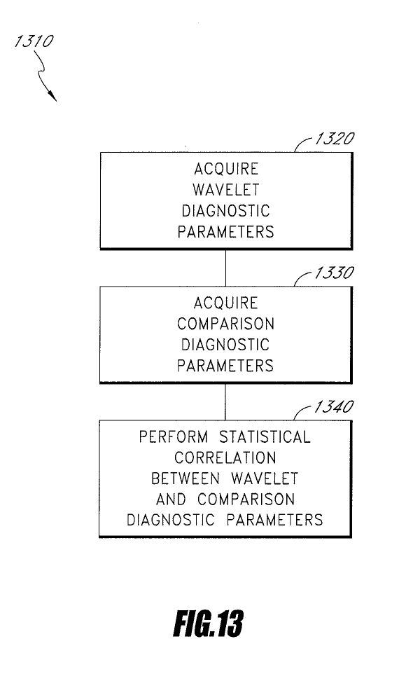

FIG. 11A illustrates a schematic diagram (a Wiggers Diagram) 1110 of a human heartbeat, which identifies the main actions of the heart cycle. The heartbeat generally may be divided into two main phases: systole and diastole. The systole is a contraction of the heart muscle that forces blood out of the ventricles, while the diastole is the relaxation of the heart muscle after the systole during which the ventricles refill with blood. During the diastole, the aortic valve closes and blood flow through the coronary arteries is at its maximum. Thus, the diastolic portion is particularly advantageous because sounds from turbulent flow generated by coronary artery occlusions should be at a maximum and because sounds from the aortic valve should be at a minimum.

FIG. 11B illustrates an example phonocardiogram 1120 of a human heartbeat. The systolic and diastolic portions are indicated. FIG. 11B shows an amplitude of an acoustic signal 1130 from the heart as a function of elapsed time. FIG. 11B shows that the amplitude of the acoustic signal is lower during the diastolic portion of the heartbeat than during the systolic portion of the heartbeat.

FIG. 11C illustrates a graph 1150 of an amplitude of an acoustic signal 1160 from a volunteer patient as a function of elapsed time. First heart sounds 1170a-1170c are caused by the closure of the atrioventricular valves at the beginning of the systolic portion of a heartbeat. Second heart sounds 1180a-1180c are caused by the closure of the aortic and pulmonic valves at the end of the systolic portion of the heartbeats. As indicated in FIG. 11C, a diastolic portion 1190 of a heartbeat begins at the second heart sound 1180a and ends at the first heart sound 1170b of the following heartbeat. FIG. 11C shows that the amplitude of the acoustic signal 1160 is relatively low during the diastolic portion of a heartbeat (see also FIG. 11B).

Accordingly, in certain embodiments of Block 940, the diastolic portion 1190 of the heart signal is selected for analysis, because acoustic signals caused by the opening and closing of the heart valves is minimized during the diastolic portion. In certain such embodiments, a central portion 1195 of the diastolic portion 1190 may be selected because of its advantageous signal-to-noise ratio or other characteristics.

In some embodiments of Block 940, the diastolic portion 1190 may be determined by the following method. The health care professional performing the measurements determines the patient's heart rate B by listening to the heart with a stethoscope. A typical value for B is about 70 beats per minute (bpm). It is preferable, but not necessary, for the heart rate B to be determined to within ±10 bpm. If the patient's heart rate is below 50 bpm or above 120 bpm, or if the heart rate is irregular (atrial fibrillation or ectopic dysrhythmia), or if the patient exhibits severe hypotension (<90 mm-Hg systolic pressure), it is inadvisable for the patient to proceed further with the measurement. In some embodiments, the health care professional may enter the heart rate B on a clinical form or hospital report for later analysis, while in other embodiments the health care professional may enter the heart rate B directly into the diagnostic device, for example, by using a keypad, a touchscreen, or a pointing device.

In some embodiments, the heart rate B may be determined by an electrocardiogram (EKG) administered contemporaneously with the acoustic measurements. In certain embodiments, the diagnostic device may comprise one or more EKG sensors used to determine the heart rate, for example, by measuring the duration between consecutive R waves in the PQRST cardiac sequence. In other embodiments, the diagnostic device may comprise one or more arterial pulse sensors configured to detect a pressure pulse in an artery, e.g., the carotid artery, so as to determine the heart rate.

A portion of the digitized acoustic signal that corresponds to one individual heart beat is selected based on the heart rate B estimated by the health care professional (or by other methods). This portion of the signal has a length L_{B} equal to the sampling rate (in Hz) divided by the heart rate (in beats per second). The maximum value of the signal in the portion is located using well-known peak finding algorithms. The location of the peak in this portion indicates the beginning of a first heartbeat.

A second heartbeat is identified as the maximum value of the signal in a range having a length L_{B} starting at a sample point 30% past the first peak and ending at a sample point 130% past the first peak. The difference in time between the first and second peaks is set to be an updated (and more accurate) estimate of the heart beat duration, and the length L_{B} is updated correspondingly. Subsequent heartbeats are determined by examining the signal for subsequent maxima. For example, in some embodiments, a range having length L_{B} starting at 60% past the previous heart beat is searched for the maximum value. The remainder of the signal may be searched until all the heartbeats have been identified. Additionally, some embodiments intercompare the values of the maxima found by this procedure to verify that the maxima correspond to heart sounds and not to noise. If a maximum is unlikely to be a heart sound, the process discussed may be iterated until a convergent result is obtained.

In certain preferred embodiments, the positions of the corresponding maxima are stored, and L_{B} is set equal to the length of the shortest heartbeat in the signal. The signals, the locations of the heartbeats, and the length L_{B} are stored prior to analysis in Blocks 950-980 of the flowchart 900. In certain such embodiments, the maxima of the signal found by this method correspond to the first heart sound. In other embodiments, a similar procedure may be used to determine the location of the second heart sound.

Having determined the location of the individual heart beats, the apparatus and methods next identify a portion of the heart signal that corresponds to the diastolic portion of the heartbeat. In some embodiments, the first and second heart sounds are used to identify the diastolic portion. The first and second sounds correspond to large peaks in acoustic amplitude (see FIGS. 11B and 11C), which may be identified by the peak-finding techniques discussed herein or by any other peak-finding algorithm suitable for time-domain signal analysis. The acoustic signal between these peaks may be extracted (e.g., by a time window), stored, and used in Blocks 950-980 of the flowchart 900.

In other embodiments, the diastolic portion of the signal is assumed to be a portion of the signal that is within a preselected range of the heartbeat length L_{B}. For example, in certain embodiments, this range may correspond to 35% to 81% of the heartbeat length L_{B}. The range may be different in different patients. In some of these embodiments, the central portion 1195 of the diastolic portion 1190 (see FIG. 11C) may be selected for further analysis due to its position away from valve sounds which mark a beginning and end of the diastole. Additionally, the turbulent acoustic signal may be largest in the central portion 1195, because the flow speed of the blood through the coronary arteries may be at its largest value in the central portion 1195. In some embodiments, the central portion 1195 corresponds to a range from 58% to 67% of the heartbeat length L_{B}. In other embodiments, different ranges may be used.

In certain embodiments, the procedure of Block 940 is applied to each sensor signal. In other embodiments, the procedure is applied to one reference sensor, and the heartbeat identifications found for the reference sensor are applied to the other sensors. In one such embodiment, the reference sensor corresponds to the sensor 1036B shown in FIGS. 10A and 10B, because it receives a signal with a higher signal-to-noise ratio due to its location above the left anterior descending artery.

In embodiments of Block 940 configured to diagnose diseases other than coronary artery disease, a portion of the received acoustic signals also may be selected for further analysis in Blocks 950-980; however, this selected portion may correspond to a different portion of the acoustic signal than the diastolic portion.

In Block 950 of the embodiment of the flowchart 900 shown in FIG. 9, the acoustic signals corresponding to a diastolic portion of a heartbeat are wavelet transformed using the methods described herein with reference to FIGS. 5-7. In some embodiments of Block 950, a diastolic portion from a single heartbeat is analyzed. In other embodiments, diastolic portions from more than one heartbeat are analyzed, which may improve a signal-to-noise ratio of the data and may lead to improved accuracy and precision. As described with reference to FIGS. 5-6D, certain preferred embodiments of the wavelet transform methods use the Morlet mother wavelet 500 to identify acoustic signals emitted by one or more stenoses in the coronary arteries.

In Block 960 of the embodiment of the flowchart 900 shown in FIG. 9, the wavelet transform of the acoustic signal is analyzed in order to detect the presence of one or more stenoses in the patient's heart. In some embodiments, the wavelet coefficients may be combined into one or more parameters that are indicative of the presence or severity of an occlusion. For example, in certain embodiments of Block 960, a wavelet diagnostic parameter (WDP) is calculated from the wavelet coefficients generated in Block 950. The wavelet diagnostic parameter is indicative of the presence and/or severity of the stenosis.

Fluid turbulence may be spatially intermittent, sporadic, and chaotic. Turbulence may also exhibit self-similarity in which one portion of the acoustic signal is statistically equivalent to another portion after appropriate rescaling. Accordingly, fluid turbulence may be characterized as a fractal process, which exhibits a self-similar statistical structure over a range of scales. A mathematical parameter known as a Hurst coefficient is a measure of a dimensionality of the fractal process. Experiments indicate that the Hurst coefficient H may be indicative of the presence and/or severity of occlusions in coronary arteries. The Hurst coefficient H can be estimated from statistics of the wavelet coefficients of the heart signals. For example, in some embodiments of the method, the variance of the wavelet coefficients at each scale is determined. In a self-similar fractal process, the Hurst coefficient H is related to the slope γ of a plot of a logarithm of the variance versus a logarithm of the scale by H=(γ−1)/2. The slope γ may be determined by a regression analysis such as, for example, a least squares analysis. See, for example, “Wavelet Applications in Medicine,” by M. Akay, IEEE Spectrum, pp. 50-56, May 1997, which is hereby incorporated by reference herein in its entirety and made a part of this specification.

In some embodiments of the apparatus and methods, the wavelet diagnostic parameter is the Hurst coefficient H, e.g., WDP=H. In other embodiments, the wavelet diagnostic parameter is a different function of the slope γ such as, for example, WDP=(\sqrt{γ}−1)/2. In other embodiments, different statistical parameters determined from the wavelet coefficients may be selected to be the WDP. In other embodiments, the wavelet coefficients may be combined in different ways to produce the WDP. Additionally, the WDP may be estimated from other signal processing coefficients such as, for example, Fourier coefficients. Other theories of turbulence may yield additional parameters other than the Hurst coefficient that are indicative of the turbulence, and these new parameters may serve as the WDP in some embodiments.

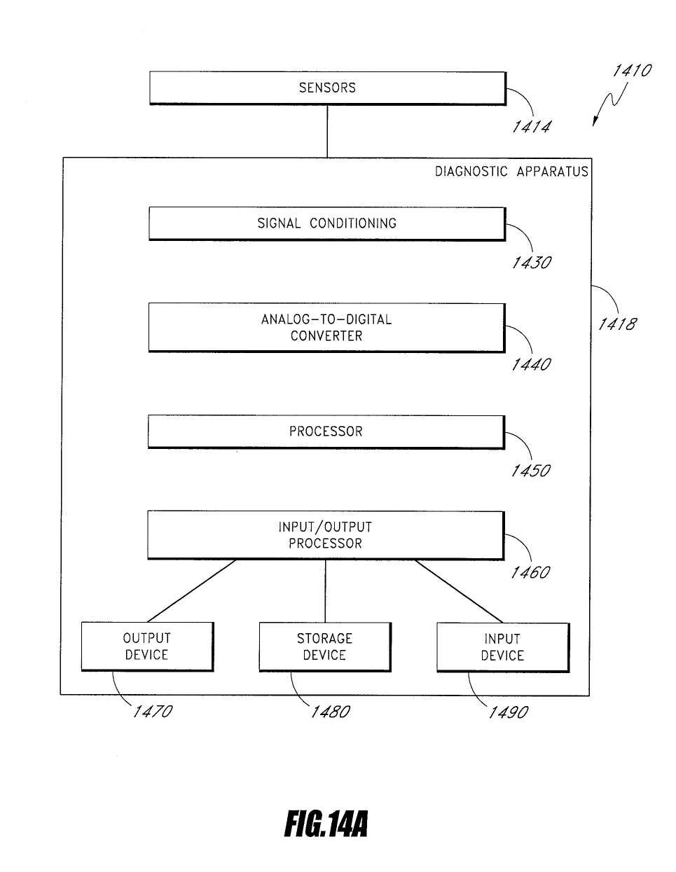

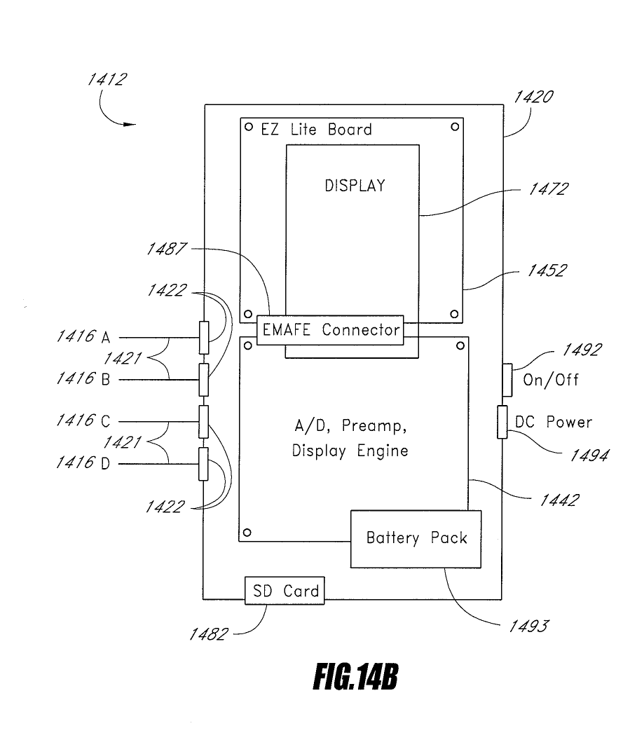

FIG. 12A is a schematic block diagram 1200 that illustrates further aspects of an embodiment of a method for diagnosing the presence of a coronary artery occlusion from acoustic data. In block diagram 1200, the digitized acoustic signal from each heart beat is analyzed in a loop comprising Blocks 1204 to 1230. In Block 1208, the diastolic portion of each heartbeat is stored. In some embodiments, the diastolic portion may be identified as described above with reference to FIGS. 11A-11C and to Block 940 of the flowchart 900 in FIG. 9. For example, the diastolic portion may be selected to correspond to a portion from 35% to 81% of the length of the heartbeat. In Block 1212, a discrete wavelet transform of the diastolic portion of the heartbeat is performed, which yields wavelet coefficients at each value the scale and translation parameters on the sample grid. For example, in certain embodiments, a sample grid similar to the example grid in Eq. (7) may be used for the discrete wavelet transform. In certain such embodiments, the scale parameters are: j=1, 2, 4, 8, 12, and 16, which correspond to frequencies of 62.5 Hz, 125 Hz, 250 Hz, 500 Hz, 750 Hz, and 1000 Hz.

In some embodiments of Block 1212, the values of the wavelet coefficients may be determined by a decimation-and-summation procedure. For example, in one embodiment, a 62.5 Hz Morlet mother wavelet is adopted as the analyzing wavelet, and this wavelet is stored in a first array having a length that is proportional to the number of samples in the diastolic portion of the signal. The length of the array is 1860 samples in one embodiment. A daughter wavelet is stored in a second array having a length equal to that of the first array divided by the scale index j. The value of the daughter wavelet is found by decimating the value of the mother wavelet stored in the first array by a factor of j. If a sample point of the daughter wavelet does not correspond to a sample point of the mother wavelet, the nearest sample point of the mother is selected. The diastolic signal to be wavelet transformed is selected to be a portion from 35% to 81% of the heartbeat and is stored in a third array. In certain embodiments, the wavelet coefficients are calculated from Eq. (6) (with the integral replaced by a sum) by translating the daughter wavelet array along the diastolic signal array and taking a dot product of the overlapping regions of the arrays. The value of the wavelet coefficient equals the value of the dot product. The dot product equals, in some embodiments, the sum of the arithmetic products of the values in the daughter wavelet array and the diastolic signal array. In certain embodiments, the dot product may be calculated using machine language multiply-and-accumulate instructions, which are computationally fast and efficient. In some embodiments for calculating wavelet coefficients, edge effects may occur for translation parameters in which the daughter wavelet array extends beyond the first or last elements in the diastolic signal array. In such cases, zero-padding of the signal array may be used. In other embodiments, edge effects are reduced, because only the central portion 1195 (FIG. 11C) of the heartbeat is used in the subsequent analysis.

The statistical variance of the wavelet coefficients is evaluated in the loop corresponding to Blocks 1216-1222 in the schematic diagram 1200. As shown in Block 1218, in some embodiments, for each value of the scale index j, only a selected range of translation parameters is used to calculate the variance. This range corresponds to the central portion 1195 (see FIG. 11C) of the heartbeat and is selected to minimize edge effects and to correspond to a suitable portion of the diastole. Wavelet coefficients may be calculated for the entire diastolic portion 1190 (see FIG. 11C), but coefficients corresponding to translation parameters outside the central range 1195 may be ignored in calculating the variance. In some embodiments, wavelet coefficients are calculated for a diastolic portion 1190 corresponding to 35% to 81% of the heartbeat length, but the central portion 1195 corresponds to a range from 58% to 67% of the heartbeat length.

In Block 1226, the slope γ of the variances is calculated. In some embodiments, the variance data is assumed to be a power law in which the variance is proportional to the scale to the power γ. Accordingly, γ may be determined as the slope of the data points in a plot of a logarithm of the variance versus a logarithm of the scale. In some embodiments, the logarithm to the base 2 is used for the slope analysis. In embodiments using a dyadic grid [e.g., Eq. (7)], the slope γ may be determined from a plot of a logarithm of the variance versus scale index j. The slope γ may be determined by standard linear regression analysis such as, for example, a least squares analysis. The slope γ is determined for each of the patient's heartbeats.

In Block 1234, the wavelet diagnostic parameter (WDP) is evaluated from the slopes found in Block 1226. As described above, in some embodiments the WDP equals the Hurst coefficient H and is calculated from (γ−1)/2. In other embodiments, different functional relationships have been found to provide useful diagnostic information. For example, in one embodiment, the WDP is found from the relationship (\sqrt{γ}−1)/2.

In Block 1234, the WDP may be determined as an average value of the WDP's determined for the individual heartbeats. For example, in some embodiments, eight heartbeats are used in the loop in Blocks 1204-1230, and the WDP is an arithmetic average of the eight individual WDP's. The use of an arithmetic average value may be advantageous in reducing inaccuracies caused by a low signal-to-noise diastolic portion in a subset of the individual heartbeats. Although eight heartbeats are used to perform the average in certain preferred embodiments, a different number of heartbeats may be used in other embodiments, and the WDP's for the individual heartbeats may be combined according to different arithmetic or statistical methods. In still other embodiments, each of the individual WDP's may be used for diagnostic purposes such as, for example, by outputting the individual values to the health care professional performing the analysis. Many variations are possible.

The number of heartbeats used in Blocks 1204-1230 may be different in different embodiments of the method described in FIG. 12A. For example, eight heartbeats have been found to reduce inaccuracies and to increase precision. However, in other embodiments, the number of heartbeats may range from one to sixteen or more. The heartbeats may come from a single measurement or from multiple measurements. It is preferable, although not necessary, that the heartbeat measurements be taken close together in time to reduce the likelihood that the patient's condition may become worse during the measurement interval. In certain embodiments, the patient is instructed to hold his breath during the measurement to minimize the acoustic signal from breathing. Accordingly, the number of heartbeats used in Blocks 1204-1230 may depend on the length of time that a patient can hold his breath. In other embodiments, the health care professional may take acoustic measurements on a number of heartbeats and may select a subset of the number of heartbeats for subsequent processing in Blocks 1204-1230. In some embodiments, the subset may be selected based on, for example, the diagnostic judgment of the health care professional, while in other embodiments, the subset may correspond to the heartbeats with the highest signal-to-noise. In other embodiments, the method 1200 may include additional Blocks in which the most suitable heartbeats are selected for further analysis.

FIG. 12B illustrates a plot 1250 of the wavelet coefficient as a function of the scale parameter and translation parameter. A horizontal axis represents the translation parameter and is measured in number of samples. Vertical axes represent the absolute value of the wavelet coefficients corresponding to the central diastolic portion 1195 of one heartbeat. Wavelet coefficients are shown for six values of the scale parameter: j=1, 2, 4, 8, 12, and 16, which correspond to frequencies of 62.5 Hz, 125 Hz, 250 Hz, 500 Hz, 750 Hz, and 1000 Hz, respectively. Wavelet coefficients, such as those illustrated in FIG. 12B, are used in Block 1218 of the diagnostic method shown in FIG. 12A and may be used for other purposes such as, for example, determination of stenosis location.

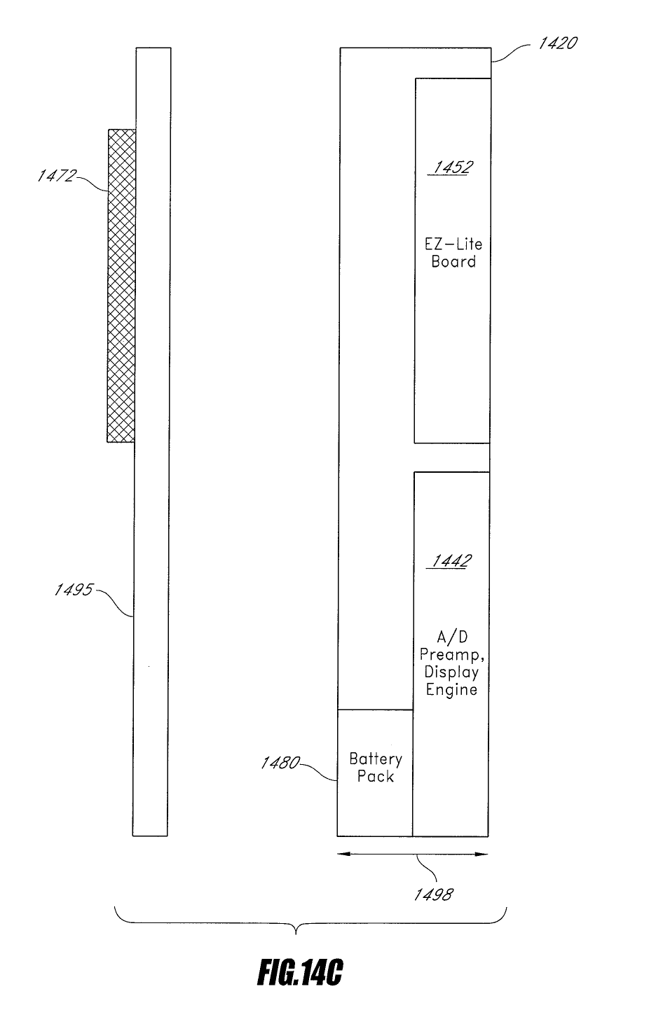

In Block 970 of the embodiment of the flowchart 900 shown in FIG. 9, the wavelet diagnostic parameter calculated in Block 960 may be used to estimate the severity of the coronary artery disease in the patient. FIG. 13 is an embodiment of a flowchart 1300 that illustrates how the wavelet diagnostic parameter is correlated with the severity of the coronary artery disease. In Block 1320, an ensemble of wavelet diagnostic parameters is acquired. The ensemble may correspond to a cohort of patients who range from healthy to unhealthy. For highest accuracy, it is preferable that the cohort represent a statistically significant group of patients. In Block 1330, one or more comparison diagnostic parameters is acquired for each of the patients in the cohort. For example, in one embodiment, the comparison diagnostic parameter is the occlusion percentage as determined by an invasive angiogram procedure. In Block 1340, a statistical analysis is performed based on the measured wavelet diagnostic parameters and the measured comparison diagnostic parameters in order to determine a statistical correlation between the parameters. For example, in one embodiment, the correlation between the wavelet diagnostic parameter and the percentage occlusion as determined by an invasive angiogram is evaluated. The results of Block 1340 may be used to correlate the wavelet diagnostic parameter with the severity of the disease. Accordingly, rather than performing an invasive comparison procedure, a health care professional may advantageously perform an embodiment of the noninvasive methods disclosed herein to determine the severity of disease.

Some embodiments of the disclosed methods may be used to as a diagnostic tool for detecting an obstruction in a coronary artery, but other embodiments can be used to identify and locate stenoses or occlusions in locations other than the coronary arteries. For example, in some embodiments, stenoses in intracranial vessels, leg vessels, and other blood vessels can be diagnosed. Some embodiments can be used to diagnose aortic aneurisms, for example. Other embodiments can be used to diagnose improperly functioning valves in arteries and veins. Certain embodiments can be used in prenatal pediatric diagnosis of fetal disease including fetal heart disease, for example.

Other embodiments may be used in conjunction with intravascular ultrasound techniques. For example, an ultrasound transducer may be inserted into an artery, and the diagnostic apparatus described herein may be used to detect and analyze the emitted ultrasound signals. Some embodiments may also be used in conjunction with other diagnostic procedures such as, for example, electrocardiograms or electroencephalograms, in order to provide the diagnostician with a more complete diagnostic analysis of the patient.

Embodiments of the acoustic methods may be used to detect acoustic signals caused by other diseases. For example, the diagnostic device can be used to diagnose pulmonary diseases in which the lung sound is modified by disease. Other embodiments can be used to detect changes in body sounds caused by tumors, cancers, or other growths.

Additionally, the wavelet analysis methods disclosed herein may be applied to acoustic signals or to non-acoustic signals. In some embodiments, wavelet analysis can be performed on electrical signals produced during an electrocardiogram or an electroencephalogram, for example.

In other embodiments, the apparatus and methods disclosed herein may be used in veterinary procedures to diagnose diseases in nonhuman animals.

Methods of Use of Preferred Embodiments

Certain embodiments of the diagnostic apparatus disclosed herein may be used to determine the presence, severity, and/or location of occlusions in coronary arteries. In some embodiments, the diagnostic apparatus can be used on male or female patients twenty one years of age or more. It is preferable, although not necessary, that a patient present clinical symptoms indicating possible acute coronary syndrome and that the patient has an ability to hold his or her breath for eight seconds, three times within five minutes. As discussed above with reference to FIG. 13, patients participating in a study comparing wavelet diagnostic results to other comparison diagnostic results such as, for example, angioplasty, may need angiography independent of their participation in the study. In addition, it may be inadvisable for patients having severe hypotension as demonstrated by blood pressure less than 90 mm-Hg systolic pressure, irregular heart rhythm (atrial fibrillation or ectopic dysrhythmia), or heart rate less than 50 bpm or greater than 120 bpm to undergo the diagnostic procedure.

The diagnostic apparatus may be used in a variety of settings such as, for example, a clinical or a hospital environment, a doctor's office, or a patient's home. Some embodiments of the diagnostic apparatus include one or more sensors, one or more electrical cables configured to connect the sensors to a diagnostic device. The diagnostic device may include an input/output unit (or separate input and output units). In certain embodiments, a person can use the diagnostic apparatus according to the following procedures, which are intended to be illustrative and not to limit the scope of possible methods of use.
1. A user of the diagnostic apparatus may prepare the patient's chest for placement of one or more sensors.
   a. Shave excessive hair if present where a sensor will be located.
   b. Provide the patient with a paper gown for privacy (if patient desires).
   c. Use topical alcohol swabs to clean the skin in the areas where sensors will be located.
2. Connect the sensors to the electrical cables.
3. Connect the electrical cables to a diagnostic device such as, for example, the device 1410, 1431, or 1510. Ensure that each sensor is connected to the correspondingly labeled sensor jack on the unit.
4. Have the patient sit in a comfortable position.
5. Remove the adhesive backing from each sensor, and apply the sensors to the patient in locations such as those shown in, for example, FIGS. 10A and 10B.
6. Ensure that a memory storage device such as, for example, a flash memory card, is inserted in the diagnostic device.
   a. In one embodiment, the flash memory card should be inserted with its label facing downward and with the cut corner pointing toward the top of the device.
   b. In one embodiment, the device will not function unless the flash memory card is properly inserted.
7. The device may be started by removing an external plug from the DC power socket. The external plug may be set aside for later use.
8. The device may include an input/output unit. For example, in one embodiment, the device includes a touchscreen, which will initially display a blank screen. The user may touch the blank screen to proceed.
9. The input/output unit will alert the user to confirm that the sensors are securely connected to both the patient and to the device. For example, in one embodiment, the touchscreen will display “Touch here when sensors are connected.”
10. Enter, on the input/output device, an ID code that identifies the device. For example, in one embodiment, the ID code is shown at the bottom of the touchscreen.
11. The device may prompt the user to begin a self-test program. For example, in one embodiment, the user touches the touchscreen to start the self-test program.
12. The device may display the connection status of the sensors on the input/output unit. When all the sensors are properly connected, the device may alert the user to begin acquiring acoustic signal data. For example, in one embodiment, a table is displayed on the touchscreen that lists each sensor along with the connection status of that sensor. If any sensors are listed as “Not Connected,” the user may check to ensure that they are securely attached to the patient and that the electrical cables are properly connected to the device. When all the sensors are listed as “Connected,” the button at the bottom will read “Touch here to begin test.”
13. Instruct the patient to hold his/her breath for eight seconds and not to move or speak.
14. Begin the diagnostic test. For example, in one embodiment, the user can tap the touchscreen at a location displaying “Touch here” to begin data acquisition.
   a. In some embodiments, the touchscreen will go completely dark for the duration of the data acquisition portion of the diagnostic test (e.g., eight seconds) once the touchscreen is touched.
15. The device may alert the user after data acquisition is complete. For example, in some embodiments, a “Processing data . . . ” message box will be displayed on the touchscreen. In some embodiments, the device may emit an audible sound.
   a. At this point, data acquisition is complete, and the patient is free to breathe, move, and speak.
16. The device may alert the user once signal processing is complete. For example, in some embodiments, the touchscreen will display a “Test Complete, Data Stored” message box.
17. After a suitable waiting time, the device will permit another diagnostic measurement to be taken and will alert the user. For example, in some embodiments, the waiting time may be about ten seconds, and a “Touch here to restart” message box will be displayed on the touchscreen.
18. In some embodiments, it is preferable, but not necessary, for the measurement test to be repeated to ensure greater accuracy and precision. For example, in some embodiments, the user may be prompted to repeat procedures 11 through 17 two more times. In other embodiments, the test may be repeated more times or not repeated at all.
19. After all sets of data have been collected, the user may determine the positions of the sensors. For example, in some embodiments, a measuring tape is supplied for the user to measure distances between the sensors as described above with reference to FIGS. 10A and 10B. In some embodiments, the user may record the sensor distances on a case report form, while in other embodiments, the user may enter the distances into the device via the touchscreen.
20. Remove the sensors from the patient.
21. Disconnect the sensors from the electrical cables and dispose of the sensors.
22. Reinsert the external plug to power off the device.
23. When the device is not in use, it may be connected to a power source, such as a battery charger. In some embodiments of the device, this may require that the external plug be removed from the DC power socket, and the battery charger inserted into a connector on the device.

Different methods of use are possible, and in other embodiments, different and/or additional procedures may be used. Further, in some embodiments the procedures may be performed in a similar order or in a different order. The methods of use may vary depending on the context of the measurements. For example, one method of use may be provided in the context of a statistical study correlating a wavelet diagnostic parameter with a comparison diagnostic parameter, while different methods of use may be provided in the context of a doctor's office, clinic, and/or hospital.

Hardware Construction and Electronics of Preferred Embodiments

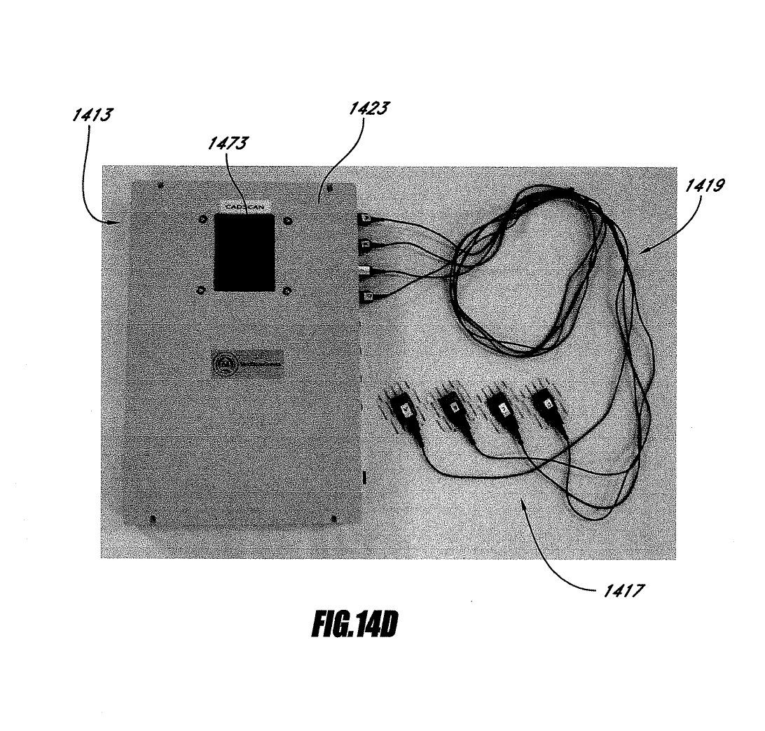

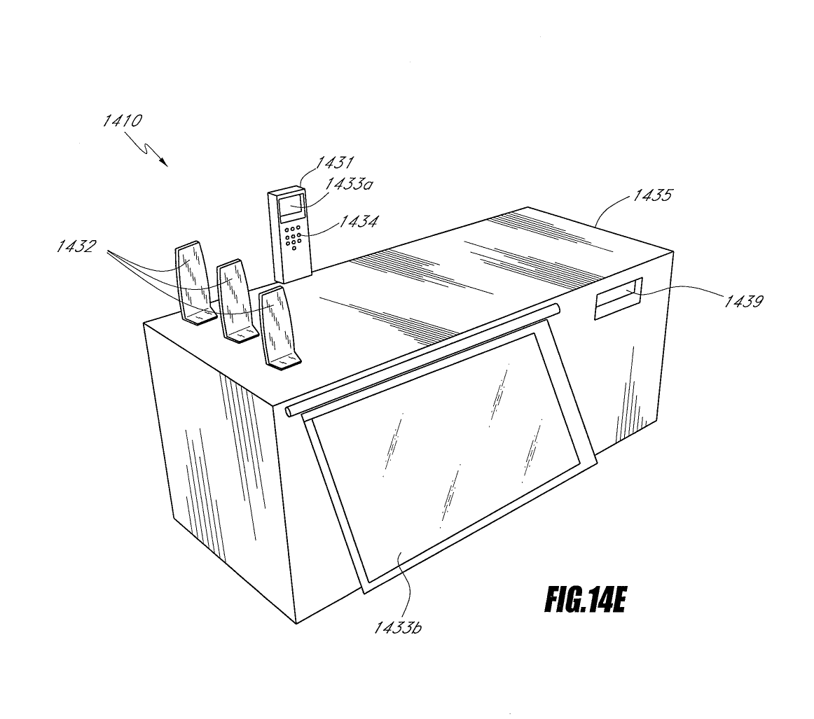

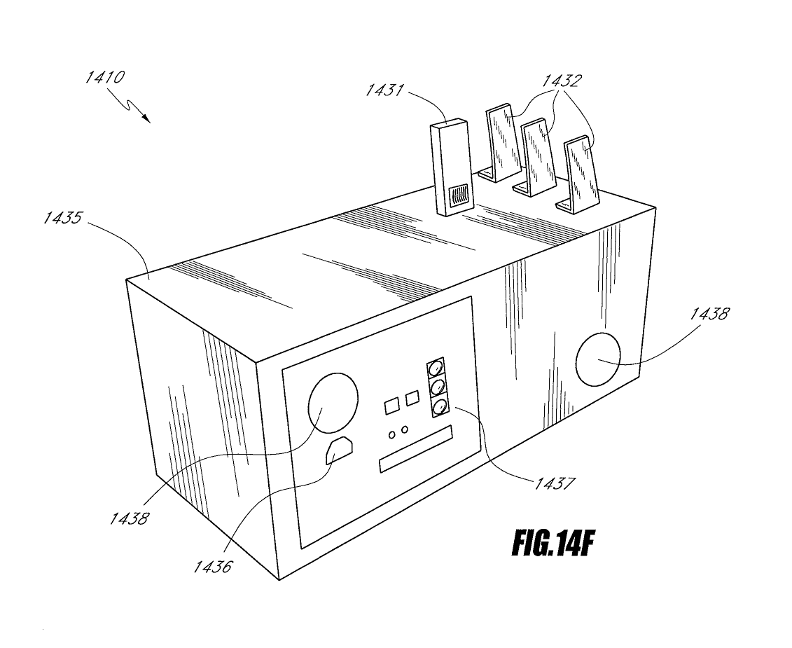

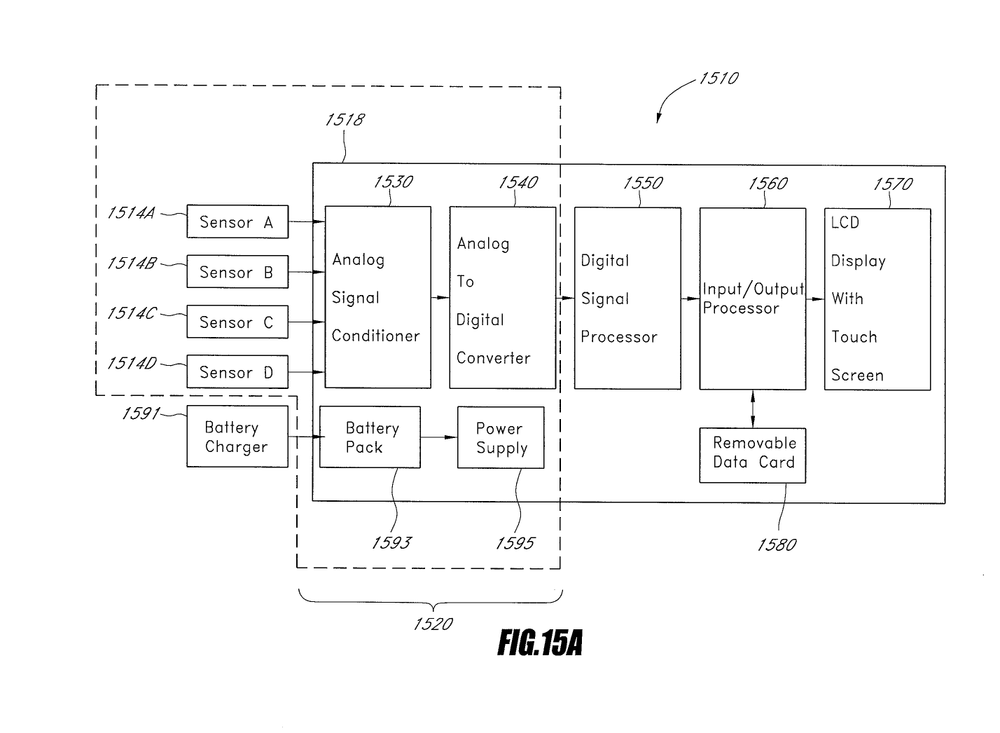

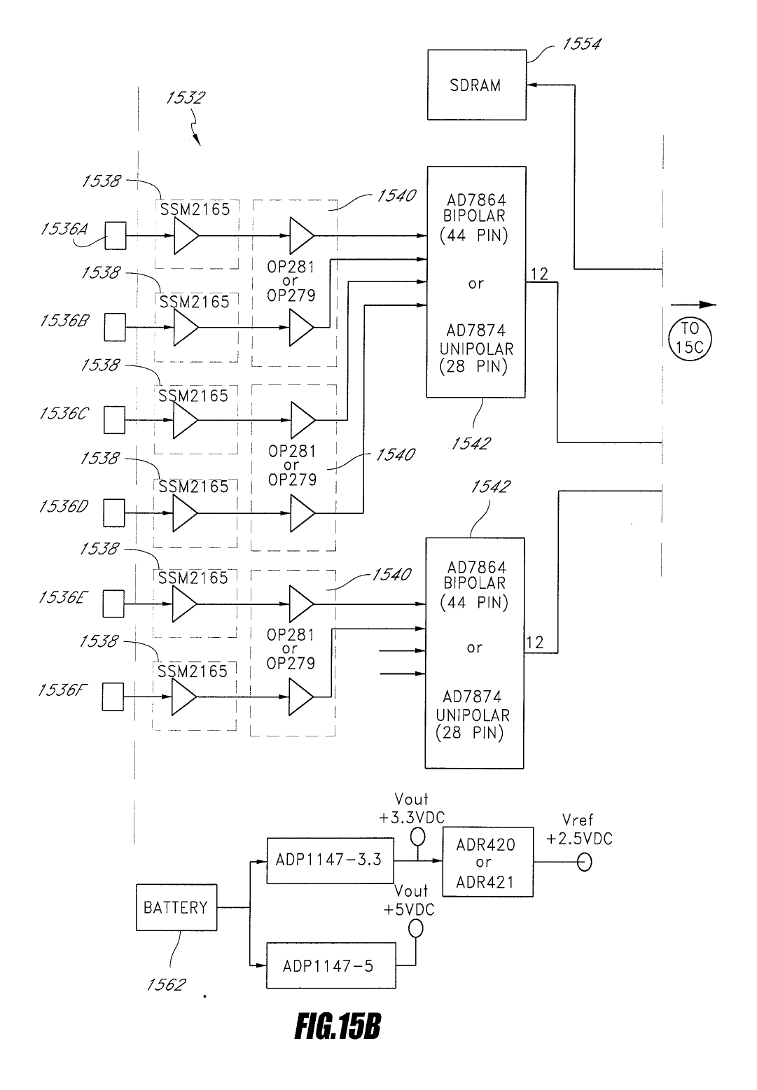

FIG. 14A is a schematic illustration of an embodiment of a device 1410 for diagnosing a biological phenomenon such as an occlusion in the coronary artery of a human. Sensors 1414 can gather data (e.g., analog acoustic data) relating to the biological phenomenon. The sensors can send the data to a diagnostic apparatus 1418 (e.g., through wired electrical connection or through a wireless connection using 802.11b radio technology or Bluetooth, for example). In some embodiments, portions that are depicted in FIG. 14A as portions of the diagnostic apparatus 1418 can instead be associated with the sensors themselves.

The diagnostic apparatus 1418 can include a signal conditioning portion 1430, an analog-to-digital (A/D) converter 1440, a processor 1450 (e.g., a digital signal processor or “DSP”), and an input/output (I/O) processor 1460. In some embodiments, the device 1410 can further comprise an external bus (not shown) coupled to the processor 1450 for coupling an external device to the processor 1450. The diagnostic tool can further comprise non-volatile memory for initialization of the processor 1450. The non-volatile memory can be built in to the processor 1450.

The subcomponents of the diagnostic apparatus 1418 can be distinct devices within a container, or they can be combined (or their processing roles shared) in many different ways. For example, the same computer chip can perform the roles of both the processor 1450 and the I/O processor 1460. In some embodiments, the A/D converter 1440 and the signal conditioning portion 1430 can be in the same chip or on the same board. In some embodiments, the diagnostic apparatus 1418 can process the signals by, for example, digitizing, filtering, synchronizing and/or multiplexing the signals, and then transmitting or communicating the processed signals to other components.

The I/O processor 1460 can interface and communicate with various devices. Such devices can include an output device 1470 (such as, e.g., a display, monitor, audio prompt, voice synthesis system, printing device, etc.); a storage device 1480 (such as, e.g., a memory card, magnetic disk or tape, flash drive, optical disk, print-out, portable hard-drive, etc.); and an input device 1490 (such as, e.g., a keyboard, mouse, touchscreen, dial, button, knob, switch, voice-recognition system, etc.) The functions of these devices that interface with an I/O processor 1460 can be combined. For example, the output device 1470 and input device 1490 can both include a touchscreen. In some embodiments, the device 1410 can comprise multiple processors and/or multiple subcomponents. For example, there may be multiple displays or multiple options for a user to obtain data from the device through various input devices 1490. The components and subcomponents illustrated in FIG. 14A can be combined and/or connected in various combinations and in diverse configurations.

In some embodiments, signal conditioning and/or analog-to-digital conversion can occur in a portable unit that is associated with the sensors, and the resulting digital signal can be sent to a separate processing unit where further processing (such as wavelet analysis) is performed. However, the device 1410 need not be portable. In some embodiments, the device 1410 can be arranged as a self-standing tool or be mountable in a housing that supports other related diagnostic tools. In some embodiments, portability is enhanced by reducing the amount of processing required for a portable unit and allowing more of the computational signal processing to be accomplished in a less-portable base station. The portable unit can thus collect and store data, which is later downloaded to the base station that processes the data. The base station can include the various components shown in the diagnostic apparatus 1418, while the sensors can additionally include memory to store the data. However, in some embodiments, signal conditioning and/or analog-to-digital conversion can be accomplished in the portable unit before being downloaded to the base station.

An embodiment of the diagnostic device 1410 that comprises a portable unit 1431 and a base station 1435 is schematically illustrated in a front perspective view (FIG. 14E) and a rear perspective view (FIG. 14F). The portable unit 1431 is configured to have a size and weight suitable for handheld use by a health care professional. The portable unit 1431 is configured to receive input signals from one or more sensors (not shown) that acquire data from the patient, such as, for example, analog acoustic data from turbulent arterial flow. The portable unit 1431 may include a power pack that provides power to the portable unit 1431. In some embodiments, the power pack comprises batteries that provide DC power to the unit 1431. In certain embodiments, the portable unit 1431 is further configured to condition the input signals and to convert the input signals into digital signals. In certain such embodiments, the signal conditioning may include amplifying and/or filtering the signals and the signal conversion may utilize an analog-to-digital converter. In some of these embodiments, the portable unit 1431 may also validate the input sensor signals by the methods described with reference to Block 920 in FIG. 9. In other embodiments, the portable unit 1431 may be configured to store and/or to transmit the digital signals. For example, the portable unit 1431 may include a nonvolatile memory device that stores the digital signals for subsequent downloading to the base station 1435. In other embodiments, the portable unit 1431 may be configured to transmit the digital signals to the base unit 1435 by wired or wireless communication.

In the embodiment shown in FIGS. 14E and 14F, the portable unit 1431 comprises a display 1433a and a keypad 1434. The keypad 1434 is used to provide input to the portable unit 1431 such as, for example, a patient identification number, patient information (age, sex, heart rate, etc.), data, time, or other suitable information. The display 1433a is used to output information such as, for example, fault codes indicating whether the sensors are properly functioning. In some embodiments, a keypad 1434 is not used, and the display 1431 comprises a touchscreen that may be used for both input and output functions.

As shown in FIGS. 14E and 14F, the base station 1435 may comprise one or more docking ports 1432 that are configured to hold the portable unit 1431 while not in use. In the embodiment shown in FIGS. 14E and 14F, four docking ports 1432 are disposed on an upper surface of the base station 1435 so that the portable units may be easily accessed by health care professionals. In FIGS. 14E and 14F, three docking ports 1423 shown on the left hand side of the base station 1435 are “empty” (e.g., they do not hold a portable unit 1431), while one docking port 1432 is “full” (e.g. it holds the portable unit 1431). The empty docking ports 1432 are available to receive additional portable units 1431 after their use is completed. In other embodiments, the base station 1435 may be configured with fewer or more docking ports 1432, and the docking ports 1432 may be disposed in different configurations, orientations, and locations relative to the base station 1435. In some embodiments, the docking ports 1432 may be separate units that are separate from the base station 1435 but which are configured to communicate with the base station 1435.

In addition to holding the portable unit 1431 while not in use, in some embodiments the docking ports 1432 may be configured to provide power to the portable unit 1431. For example, in some embodiments, a rechargeable power pack may be disposed within the unit 1431, which advantageously can be recharged while disposed in the docking port 1432. In certain preferred embodiments, an electrical interlock system prevents the portable unit 1431 from being used for patient measurements when the unit 1431 is attached to the docking port 1432 so as to prevent an electrical shock or current from reaching the patient. In these embodiments, the portable unit 1431 must be completely detached from the docking port 1432 (and therefore electrically disconnected from the base station 1435) before the interlock system will permit measurements to be taken. In further embodiments, the docking ports 1432 are configured so that digital signal data may be downloaded from the portable unit 1431 into the base station 1435 for additional processing, analysis, and/or storage. For example, in the embodiment shown in FIGS. 14E and 14F, the digital signal is transferred from the portable unit 1431 by an electrical connection included in the docking ports 1432. In other embodiments, the digital signal may be transferred to the base station 1435 via a wireless communications network.

In other embodiments, the portable unit 1431 may include a radio frequency identification (RFID) device, which can be used to communicate to the base station 1435 information such as, for example, a unique identification code assigned to each portable unit 1431. Certain embodiments advantageously use the identification code to ensure integrity, security, and privacy of the measurements taken by the portable unit 1431 and downloaded into the base station 1435.

In some embodiments, the base station 1435 comprises a housing 1439 that includes the electronics used for analyzing and processing the digital signals. For example, the base station 1435 may include a processor that performs a wavelet transform of the digital signal so as to produce a wavelet diagnostic parameter indicative of the presence or severity of a disease such as, for example, coronary heart disease. As shown in FIG. 14E, the base station 1435 includes a display 1433b that may be used for visualization of the results of the processing. For example, the display 1433b may illustrate textual or graphical indications of the presence, severity, and/or location of coronary artery occlusions. In some embodiments, the base station 1435 may communicate the wavelet diagnostic parameter (or other relevant diagnostic information) to the portable unit 1431 for output on the display 1433a. As shown in FIG. 14E, the display 1433b may be pivotably attached to the base station 1435 so that the display 1433b can be suitably oriented for ease of use. The base station 1435 may be configured to be a self-standing tool or to be mountable in a housing that supports other diagnostic tools.

FIG. 14F schematically illustrates a rear perspective view of an embodiment of the device 1410 showing an AC power connector 1436, cooling fan output vents 1438, and a connector panel 1437. The connector panel 1437 may be configured to couple one or more peripheral device ports to an external bus that communicates with the electronics disposed within the housing 1439. The peripheral device ports may include, for example, serial ports, parallel ports, universal serial bus (USB) ports, IEEE 1394 (FireWire) ports. Various peripheral devices may be coupled to the base station 1435 through the connector panel 1437 including, for example, a keyboard, a mouse, a hard drive, an optical drive, a printer, a plotter, a display, a scanner, or other suitable device. The connector panel 1437 may also be configured to include a memory card reader, a floppy drive, an optical drive (e.g., a CD-ROM drive or a DVD drive), or other suitable component.

In other embodiments of the diagnostic device 1410 shown in FIGS. 14E and 14F, the signal acquisition and signal analysis tasks may be shared differently. For example, in one embodiment, the portable unit 1431 may perform the wavelet transform of the digital signals and may communicate the wavelet coefficients to the base station 1435 for further processing into a wavelet diagnostic parameter. In other embodiments, the portable unit 1431 may encrypt the digital signals prior to communicating the signals to the base station 1435 in order to increase security and privacy of patient data and results. Other variations and configurations are possible, and FIGS. 14E and 14F are not intended to limit the range of embodiments of a diagnostic device 1410 comprising a portable unit 1431 and a base station 1435.

FIG. 14B schematically illustrates one embodiment 1412 of an apparatus for detecting occlusions in the coronary arteries. The diagnostic apparatus 1412 is an example of the generalized device 1410 of FIG. 14A. Four acoustic sensors are labeled 1416A, 1416B, 1416C, and 1416D. The sensors 1416A-1416D are responsive to the acoustic energy emitted by an organ or other biological entity such as the heart, which can comprise an acoustic signal from a stenosis. The sensors can be shielded from ambient noise and configured to sense acoustic signals emanating from within the body. For example, in some embodiments, the sensors 1416A-1416D are acoustically coupled to the skin of the patient. As further described herein with reference to FIG. 8, each sensor detects the analog acoustic energy and transmits a signal representative of the acoustic energy to additional hardware/software components for signal processing. In some embodiments, the sensors 1416A-1416D transmit an analog signal that is sampled and digitized by other components such as, for example, an analog-to-digital converter 1442. In other embodiments, the sensors 1416A-1416D transmit a digitized signal. In certain embodiments, the sensors 1416A-1416D comprise ultrasound transducers, which may comprise ultrasound transmitters, receivers, microphones, and/or piezoelectric devices.

Sensors suitable for use with the systems and methods disclosed herein include, for example, an Androsonix biological sound sensor such as a model BM20A322P01 acoustic transducer (Andromed, Inc., Quebec, Canada). In other embodiments, the sensors comprise Andromed, Inc. transducers that have been cleared for marketing through a premarket notification (K021389: Oct. 1, 2005) as a “Biological Sound Monitor Sensor.”

In certain preferred embodiments, it is advantageous for the sensors to be responsive to acoustic signals arriving from a wide range of directions. Additionally, for sensors that comprise more than one layer of acoustically sensitive material, it is advantageous for the layers to be separated by a distance sufficiently small that the acoustic signal arrives at each layer at substantially the same time. Such suitable sensors may include the acoustic sensors disclosed in U.S. Patent Application No. 60/692,515, entitled “Acoustic Sensor,” filed Jun. 21, 2005, which is hereby incorporated by reference herein in its entirety and made a part of this specification.

In some embodiments, suitable sensors include the sensors disclosed in U.S. Pat. No. 5,885,222, entitled “Disposable Acoustic Pad Sensors,” issued Mar. 23, 1999 to Kassal et al., the entire disclosure of which is hereby incorporated by reference herein and made a part of the this specification. Another suitable sensor configuration is described in U.S. Pat. No. 5,365,937, entitled “Disposable Sensing Device with Contaneous Conformance,” issued Nov. 22, 1994 to Reeves et al., the entire disclosure of which is hereby incorporated by reference herein and made a part of the this specification.

In certain embodiments, the sensors are configured to include a processing element, which may include a microchip, a microprocessor, a radio frequency identification device (RFID), or other processing device. The processing element may advantageously be used to provide signal preprocessing before transmission to the diagnostic apparatus 1420. Additionally and optionally, the processing element may be used to interface with other components or devices to provide information related to sensor identification, location, validation, or calibration.

In certain embodiments, the diagnostic apparatus 1420 may be provided to a patient for home use and self monitoring. In such embodiments, the sensors 1416A-1416D may be configured to be worn by the patient for a period of time. At various time intervals, the patient may take self-tests of his or her condition by, for example, connecting the sensors 1416A-1416D to the diagnostic apparatus 1420 and performing an acoustic measurement. The results of the self-tests may be stored by the diagnostic apparatus 1420, or the results may be transmitted to a hospital, doctor, or diagnostician for analysis.

In some embodiments, each acoustic sensor 1416A-1416D is connected to the diagnostic apparatus 1420 through cables 1421 that allow electrical signals to pass between the sensors 1416A-1416D and other components of the diagnostic apparatus 1420. For example, electrical signals can convey acoustic data corresponding to vibrations detected by sensors 1416A-1416D. The cables 1421 are advantageously flexible and long enough to extend between the device 1412 and the patient. The cables 1421 can be shielded to preserve the integrity of the electrical signals that pass between the sensors 1416 and the diagnostic apparatus 1420. Although some described embodiments include four sensors, more or fewer sensors can be used. In certain embodiments, the sensors 1416A-1416D may be configured to communicate with the diagnostic apparatus 1420 via a wireless communications protocol or via an optoelectronic protocol.

The device 1412 further includes connectors 1422 that allow the cables 1421 to connect with the circuitry inside the device 1412. The circuitry can include a diagnostic apparatus 1420 that is an example of the generalized diagnostic apparatus 1418 of FIG. 14A. The illustrated diagnostic apparatus 1420 comprises two different circuit boards for signal processing. The first circuit board 1442 combines some signal conditioning functions, analog-to-digital conversion of the signal, and additionally provides the processing power required to drive the display 1472. Thus, the first circuit board 1442 is an example of a combined component performing the functions of the signal conditioning unit 1430, the A/D converter 1440, and the I/O processor 1460—each of FIG. 14A—all on the same circuit board.

In some embodiments, the display 1472 is a touchscreen that can also perform the function of an input device 1490. When functioning as a touchscreen, the display 1472 can send signals to and receive signals from the first circuit board 1442, as appropriate. Thus, the display 1472 can allows a user to control and/or interact with the diagnostic apparatus 1418.

The second circuit board 1452 provides digital signal processing power that may be needed to analyze the data using, for example, the wavelet transform mathematics discussed above. Thus, the second circuit board 1452 is an example of the processor 1450 of FIG. 14A. The second circuit board 1452 can be an EZ-Lite board, available from EZ-Labs of Yonkers, N.Y. (ez-labs.com). However, other circuit boards can also be employed. In some embodiments, the system runs C, C++, Visual DSP++, MATLAB®, Maple®, Mathematica® BASIC, FORTRAN, Pascal, JAVA, or another programming language. Thus, the circuit board 1452 can perform the calculations and operations described in the flow charts above to process the data signals from acoustic sensors 1416A-1416D. In other embodiments, the instructions for performing the signal analysis may be included in software, hardware, or firmware modules.

A connector 1487 can connect the first circuit board 1442 to the second circuit board 1452. The connector 1487 can be an enhanced modular analog front end (EMAFE) connector that allows electrical signals and data from multiple channels to pass between the two circuit boards 1442 and 1452.

Moreover, device 1412 includes an “on/off” control 1492 that can complete a circuit allowing direct or alternating electrical current to flow through the device 1412. In some embodiments, the electrical power is in direct current (DC) form, supplied by a battery pack 1493. The battery pack 1493 can provide the device with portability and can reduce or eliminate the need for plugging the device into an electrical grid. In some embodiments, the device 1412 can be powered through a jack 1494 for DC power. The DC power can allow the battery pack 1493 to recharge, improving the portability of the device. For example, in some embodiments, the device can be a portable hand-held device that can be recharged by placing it in a recharging cradle when not in use. In some embodiments, the device 1412 (or a portion thereof) is designed to shut itself off automatically to minimize energy use. For example, the device may shut itself off or switch to a lower power usage after the device is not used for a certain time period. Such a period can be five minutes, for example. In some embodiments, the user can change the settings of the device to lengthen or shorten the time before such an inactive status is automatically triggered.

The battery pack 1493 may comprise any type of electrical storage device or portable power generation technology. For example, some embodiments use batteries that are single-use disposable units, while others use rechargeable units. The battery pack 1493 may comprise, in various embodiments, alkaline, nickel-cadmium (NiCad), nickel-metal hydride (NiMH), lithium ion, or other types of batteries. The battery pack 1493 may be configured to have a capacity to take measurements for a time period (such as, for example, one day) or for a number of patients (such as, for example, a typical number of patients seen by the health care professional during a shift). In embodiments of the device 1412 that comprise a portable unit configured to take measurements and a less portable, base station configured to perform analysis functions, the portable unit may be configured to be recharged while disposed on or within the base station. Additionally in such embodiments, data measurements may be downloaded while the portable unit is disposed on or within the base station. In some embodiments, the portable unit may be disposed in a docking or recharging cradle, which is configured to communicate with the base station.

The battery pack 1493 may be configured to comprise a removable battery unit so that a discharged battery unit may be removed and replaced with a fully-charged battery unit. In certain embodiments, the battery pack 1493 may comprise a photovoltaic device, such as a solar cell, which may be configured to provide sufficient power to the apparatus 1420 from ambient light sources, such as room light. In other embodiments, other power generation technologies may be used such as, for example, electrochemical devices, fuel cells, mechanical or wind-up power sources, etc.

The device 1412 may comprise a backup power source such as, for example, an uninterruptible power supply (UPS), which may advantageously be used to permit measurements to be taken and analysis to be performed during power outages. The device 1412 may also comprise a universal power adapter configured to permit the use of a wide range of internationally available input voltages (e.g., from 110-240 volts and from 50-60 Hz AC).

As illustrated in FIG. 14B, in some embodiments, the storage device 1480 may comprise a memory card such as, for example, a secure digital (SD) card 1482 configured to connect with the diagnostic apparatus 1420 through a slot in the housing. The SD card 1482 can comprise nonvolatile memory, for example, and can be disconnected from the diagnostic apparatus 1420. The SD card 1482 may be connected to other devices so as to transfer patient data or results to the other devices for purposes such as, for example, data storage, archiving, or processing. In other embodiments, the memory card 1482 may comprise other types of volatile or nonvolatile memory devices or flash memory devices. In certain embodiments, the memory card 1482 may comprise secure digital (SD), compact flash (CF), memory stick (MS), multimedia card (MMC), xD-Picture card (xD), or SmartMedia (SM) card. In various embodiments, the memory card 1482 may comprise an electrically-erasable programmable read-only memory (EEPROM) or a nonvolatile read-write memory (NVRWM) or any other type of semiconductor memory. In other embodiments, other types of storage devices may be used such as, for example, battery-backed random access memory, magnetic random access memory (MRAM), bubble memory, mini-hard disk, or microelectromechanical systems (MEMS) memory device. In yet other embodiments, the device 1412 is configured to communicate with other devices via fiber optic or cable connections.

In some embodiments, the device 1412 stores patient data in a medium that allows it to be retrieved in the future. This medium may be, for example, a flash memory or other portable memory device that permits the user to transfer patient data or results to a database in a storage medium or to another diagnostic device or to a data network. In some embodiments, the data/results are transmitted via a wired connection (e.g., metal wires, cables, fiber optics, land-based telephone lines, modems, etc.) In other embodiments, the patient data or results may be transmitted wirelessly to a database or network using, for example, Bluetooth wireless technology or another wireless, cellular, or satellite transmission protocol. The wireless technology may include terrestrial and/or satellite signal transmissions, and the wireless communications may occur via narrowband or broadband signals. Networks may comprise a local area network (LAN) or a wide area network (WAN). In certain embodiments, the device 1412 may be configured to perform both wired and wireless communication. The device 1412 may, in some embodiments, transmit the acquired patient data in real-time, while in other embodiments, it may transmit the data at a later time, which may depend on, for example, available network bandwidth and/or network or analysis queuing protocols.

In some embodiments, the device 1412 may be configured so that a portable unit performs signal acquisition and measurement functions, while a less portable, base station performs signal analysis functions (e.g., calculating the wavelet diagnostic parameter). In such embodiments, the device 1412 may be configured so that the portable unit communicates with the base station, and the base station communicates with a storage medium, data network, or information system. In certain such embodiments, the portable unit may be configured to include a radio frequency identification (RFID) device, which may provide, for example, device identification data, location data, tracking data, etc. The RFID device may increase security by permitting only registered portable units to communicate with the base station.

Patient data and/or measurement results may be stored in a patient database, which can permit the user to compare the data or results to previous measurements. The patient data or results may be stored on local or remote device, network, or node. For example, in one embodiment, the patient data or results are communicated to a Hospital Information System (HIS) where the data or results may be shared with other health care professionals attending the patient. In some embodiments, the device 1412 may be calibrated according to information in a database or HIS for differing patient age groups and body types. Such information may be stored on a flash memory card, for example, or on an external database. The device 1412 may allow the user to compare diagnostic results across age groups and body types, for example. The diagnostic results may be encrypted to provide increased security.

In some embodiments, the device 1412 includes the capability of outputting patient measurements or results to a graphical display device such as, for example, a printer, a plotter, or a display. Data from the device 1412 can be sent to the graphical display device through a wireless network, through a cable comprising a parallel or serial port, a universal serial bus (USB), or a IEEE 1394 (e.g., FireWire) connection, or through a portable memory device, for example. The output of the device 1412 may, for example, be presented on a number or letter scale, or it may be presented in a color-coded format to indicate the severity of occlusions, and/or it may be formatted to give a two-dimensional or three-dimensional representation of the results of the scan. The results may be displayed simultaneously with a representation of the internal or external anatomy of the patient. In some embodiments, the device 1412 is capable of producing a description of the location of any occlusions in an appropriate language. Such a description may, for example, be a clinical text description, which the device 1412 could automatically produce at the conclusion of the scan. In some embodiments, the device 1412 may generate an acoustic output, such as a tone, bell, auditory signal, or may use a voice synthesis system to provide patient information.

The graphical display device may comprise a printer such as, for example, a laser printer, an inkjet printer, a thermal printer, or other device configured to provide a tangible record corresponding to the patient data or results. The graphical display device also may comprise a display unit such as, for example, a monitor, a cathode ray tube (CRT) display, a liquid crystal display (LCD), a light emitting diode (LED) device, a MEMS display, or other monochrome, gray scale, or color display device. In embodiments of the device 1412 comprising a portable unit and a base station, either unit or both may be configured to include a graphical display device, each of which may be configured to output data in the same or in different formats. For example, the portable unit may output information related to signal acquisition and signal validation, while the base station may output patient diagnostic information such as the wavelet diagnostic parameter or occlusion location.

The graphical display device may output data in text and/or graphical formats. In some embodiments, the device 1412 is configured to provide data in a standard industry format such as, for example, portable document format (PDF), hypertext markup language (HTML), ASCII, rich text format (RTF), Microsoft® Word® or Office® format, joint photographic experts group (JPEG), graphics interchange format (GIF), portable network graphics (PNG), bitmap (BMP), etc. Patient data or results may be output in formats suitable for inclusion in other suitable programs such as, for example, database programs (e.g., formats using structured query language (SQL) or Microsoft® Access®), mathematical analysis programs (e.g., MATLAB®, Maple®, or Mathematica®), computer graphics programs (e.g., Autodesk® AutoCAD®, Microsoft® PowerPoint® or Visio®), or other industry standard or proprietary programs. In certain embodiments, the graphical output may be in a form suitable for use by medical insurance companies.

Graphical formats may include two- and three-dimensional visualization protocols. In some embodiments, the graphical display device may output patient data or results as a movie or a video in such formats as, for example, moving picture experts group format (MPEG), audio video interleave format (AVI), Apple® QuickTime® format, or other suitable industry or proprietary formats.

In some embodiments, the graphical display device may be configured to generate a variety of graphics, which can be used to provide suitable visualizations of the patient data or results. In one embodiment, the graphical display device may output data in a format suitable for use by the patient and/or in a format suitable for use by a doctor, clinician, diagnostician, or health care professional. For example, a graph showing the time history of the severity of the patient's occlusion may be useful for monitoring the efficacy of a disease reduction regimen. Additionally, the graphics may show correlations of the patient data or results together with other diagnostic parameters. For example, in one embodiment, a graph may show the patient's wavelet diagnostic parameter, heart rate, body mass index, occlusion location, etc. In embodiments which store or have access to data from other patients, the graphical display device may show, for example, a plurality of wavelet diagnostic parameters from all members of a suitable patient statistics cohort. Such data may be used advantageously to track treatment outcomes for the members in the cohort.

In some embodiments, the device 1412 is compatible with other diagnostic technologies so that results from the diagnostic apparatus 1420 can be incorporated into information obtained from other procedures in order to obtain a better diagnosis. Other diagnostic technologies that may be suitable for use in addition to the methods discussed herein include, for example, magnetic resonance imaging (MRI), computer aided tomography (CAT), positron emission tomography (PET), X-rays, ultrasound, cardiograms, electroencephalograms, blood pressure, blood chemistry, stress tests, and/or body mass index (BMI). Other diagnostic procedures may be utilized as well.

FIG. 14C shows a schematic, cross-sectional side view of the apparatus 1412 of FIG. 14B. FIG. 14C shows the relative positions of various components that were shown schematically superimposed in FIG. 14B. The diagnostic apparatus 1420 can have a thickness 1498 of approximately 2 inches in one embodiment. A removable cover 1495 to which the display 1472 is attached is shown separated from the rest of the body of the device 1412.

FIG. 14D is a photograph of an embodiment of a diagnostic device 1413 including a diagnostic apparatus 1423, a display 1473, cables 1419, and acoustic sensors 1417. The diagnostic apparatus 1421 can be generally contained within and protected by a housing such as a strong but portable metal or plastic box, for example.

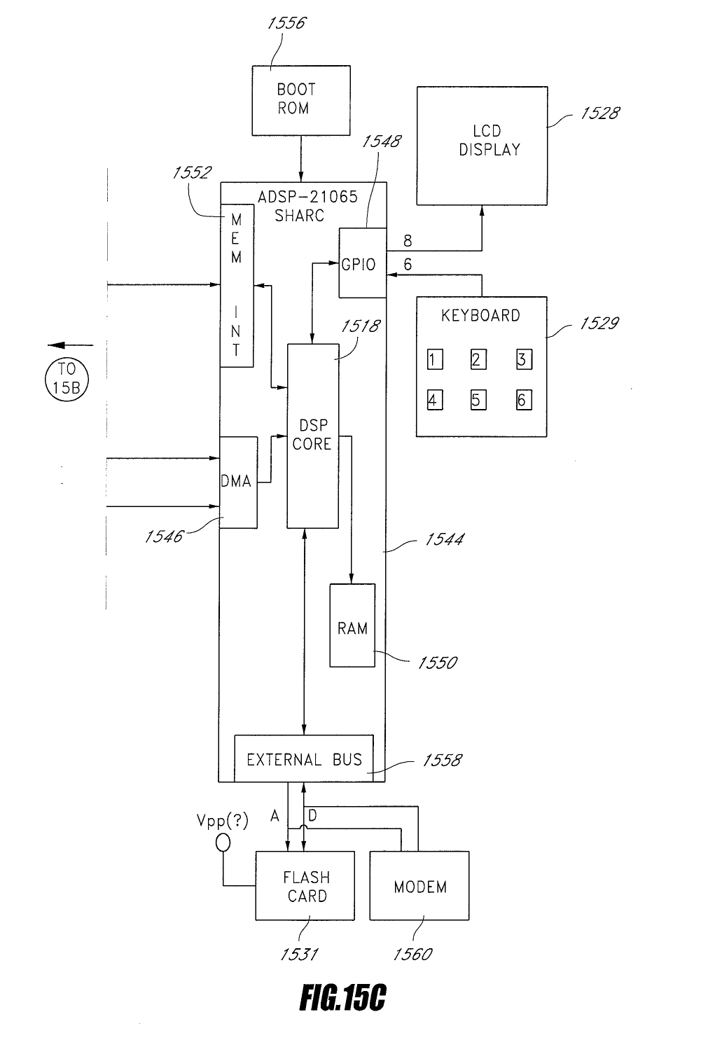

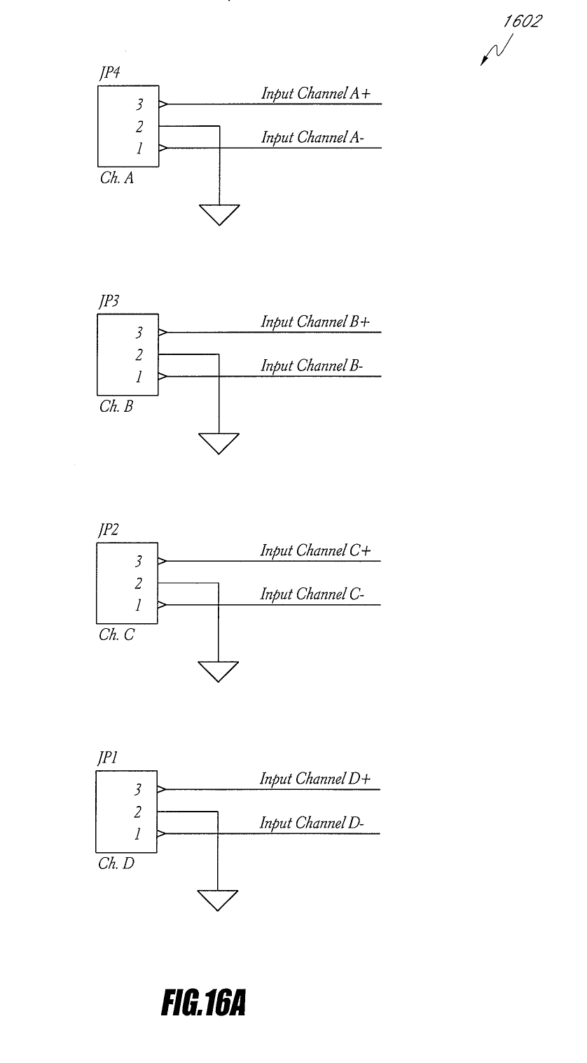

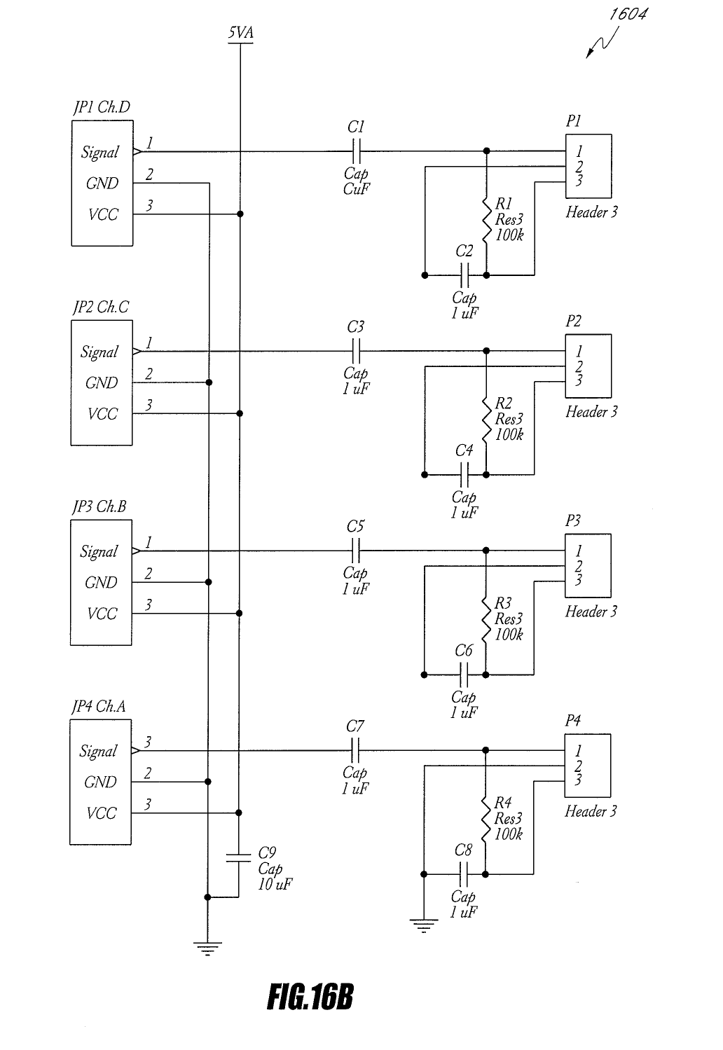

FIG. 15A is a schematic illustration of an embodiment of a device 1510 for diagnosing a biological phenomenon. Various subcomponents are illustrated. FIG. 15A is also a flow chart showing how signals and processes can occur in a sequence and signals can be transmitted to various components in a certain way.

The device 1510 includes four sensors 1514A-1514D; an analog signal conditioner 1530; an analog-to-digital converter (ADC) 1540; an digital signal processor (DSP) 1550; an input/output processor (I/O processor) 1560; an LCD display with touchscreen 1570; a removable data card 1580; a battery charger 1591; a battery pack 1593; and a power supply 1595. In the embodiment shown in FIG. 15A, all components except the battery charger 1591 and the sensors 1514A-1514D are part of a diagnostic apparatus 1518.

In certain embodiments, the device 1510 may be configured and may function in accordance with the following. The sensors 1514A-1514D are piezoelectric PVDF acoustic sensors that are attached to a subject's skin with a biocompatible adhesive. The sensors 1514A-1514D convert the acoustic energy from the body into an analog electrical signal for further processing. The analog signal conditioner 1530 receives the analog electrical signals from the sensors, filters them with a low pass filter anti-aliasing filter and amplifies them.

The analog-to-digital converter 1540 takes the conditioned analog signals and samples them at a sampling rate so as generate digital signals. In certain embodiments, the sampling rate may be, for example, 2 kHz, 4 kHz, 5 kHz, 22 kHz, 44 kHz, 120 kHz, 500 kHz, 1 MHz, or other suitable sampling rate. It is preferable, although not necessary, for the sampling rate to be sufficiently large that the low pass filtered analog signal is Nyquist sampled, e.g., sampled at a rate greater than or equal to twice a maximum frequency present in the low pass filtered analog signal. During this process all four signals are sampled simultaneously and then made available on a data bus (not shown) for further processing. In other embodiments, the four signals may be sampled sequentially.

The digital signal processor 1550 may be used to define the sample rate for the analog to digital converter 1540, to move the data into volatile memory (which can be part of the DSP 1550), and to perform algorithms to validate that the sensors 1514A-1514D are connected, functioning and sensing a heartbeat. The DSP 1550 applies another low pass filter to the digital signal, such as, for example, a digital FIR filter as further described with reference to Block 930 of FIG. 9, and parses the digital signal into single heartbeat diastolic periods for wavelet transform analysis. Once the wavelet analysis is complete, a wavelet diagnostic parameter is generated based on the presence or absence of frequencies that are indicative of turbulence in the arterial blood flows.

The input/output processor 1560 coordinates the system operation through the LCD display 1570. The I/O processor 1560 may include a graphical user interface (GUI), which can format the graphical output in an informative and useful manner. Upon power up (which can correspond to removal of an input to the battery charger 1591), the processor checks to ensure a removable data card 1580 (e.g., a nonvolatile or flash memory card) is installed in the system. If the data card 1580 is not present, an error message is displayed on the LCD display 1570 instructing the user to insert a card. Once a card is present, various graphical or text messages are sent to the LCD display 1570, and user touchscreen responses are processed to coordinate data collection and storage. The I/O processor 1560 also assigns an identifier to each data set being recorded that consists of the serial number of the unit and an incremental record number. Additionally, the I/O processor 1560 stores the raw data and any processed results from the digital signal processor 1550 into nonvolatile memory on the removable data card 1580. The I/O processor 1560 also monitors the signals from sensors 1514A-1514D for inactivity and will go into a power save mode after a suitable time, such as ten minutes. Touchscreen activity will restart the unit after power save mode has been entered.

The LCD display with touchscreen 1570 provides visual instructions and information for the user generated by the I/O processor 1560. It also takes tactile responses from the user and provides them to the I/O processor 1560.

In some embodiments, the removable data card 1580 comprises a flash memory-based device that receives the data from the I/O processor 1560 and stores the data. The card 1580 is removed from the device 1510 for the transfer of data to a mass storage system (not shown) and for possible further analysis and archiving of the data.

In some embodiments, the illustrated components can comprise a portable unit 1520, which may be configured to have a size and weight suitable for handheld use. The battery pack 1593 may comprise lithium ion batteries to provide electrical power to the device 1510. If the device 1510 includes a portable unit 1520, various mechanisms that prevent electric shock to a user can be advantageous. For example, in some embodiments, there is an interlock system that precludes the portable unit 1520 (and/or the device 1510) from performing signal measurements while the battery charger 1591 is connected. In these embodiments, the portable unit 1520 must be disconnected from the battery charger 1591 before the power supply 1595 will energize the unit 1520 in preparation for signal acquisition. The battery pack 1593 includes protection against overcharging, excessive current drain, and low voltage to stop further discharging of the battery pack 1593.

The power supply 1595 takes the electrical power from the battery pack 1593 and conditions it for use by all components of the portable unit 1520. The power supply 1595 can comprise various voltage regulators to provide the required voltages for the components. The battery charger 1591 can be specifically designed to provide the appropriate voltage and current for charging the battery pack 1593. The power supply 1520 may be configured to accept AC voltage and may include a universal power adapter configured to accept suitable international AC voltage combinations. The device 1510 may include interlocks to prevent power from flowing to the portable unit 1520 while the portable unit 1520 is being used to measure patient signals. Such interlocks prevent electrical shocks or excessive electrical current from reaching the patient.

FIGS. 15B and 15C show a schematic illustration of the electronics for a processing unit 1532 that can function similarly to the device 1410 of FIG. 14A and/or the device 1510 of FIG. 15A. However, in this alternative embodiment, some components may be different. For example, in this embodiment, six acoustic sensors 1536A-1536F are coupled via cables 1538 to the processing unit 1532. In some embodiments, the sensors 1536A-1536F are ultrasonic patches adhered to the chest of a patient for monitoring the heart beats of the patient and transmitting signals indicative of the heart sounds.

A pre-amplifier 1538 can be coupled to each of the sensors 1536A-1536F for amplifying the signal received from the sensors 1536A-1536F and transmitting the amplified signals to a plurality of operational amplifiers 1540. In the illustrated embodiment, the operational amplifiers 1540 are single ended low noise amplifiers having a frequency response that is flat to 1 kHz with a nominal gain of approximately 18 decibels. The operational amplifiers include outputs coupled to at least one analog to digital converter 1542. The analog to digital converters 1542 are for at least one of digitizing, multiplexing, synchronizing and localizing of the signals received from the operational amplifiers 1540 and for transmitting the digital signals to a digital signal processor unit 1544 via a dynamic memory access (DMA) chip 1546. As shown in FIG. 15B by way of example, the analog to digital converters 1542 are Analog Devices® AD 7864 or AD 7874.

The digital signal processor unit 1544 includes a digital signal processor core (DSP core) 1518 coupled to the analog to digital converters 1542 for processing the signals received from the sensors 1536A-1536F. The digital signal processor unit 1544 comprises an Analog Devices® ADSP-21065 32-bit floating point DSP in one embodiment. The DSP core 1518 is coupled to the display 1528 and keyboard 1529 via a general purpose input/output interface (GPIO) 1548. The processing unit 1544 also includes random access memory (RAM) 1550 coupled to the DSP core 1518 as well as an SDRAM interface 1554 for coupling the DSP core 1518 to SDRAM memory 1554. A Read Only Memory (ROM) 1556 is coupled to the DSP core 1518 for storing start-up or boot instructions for the DSP core 1518. An external bus 1558 is coupled to the DSP core 1518 for coupling the flash card 1531 to the DSP core 1518 as well as a modem 1560. Both the flash card 1531 and the modem 1560 are provided for transferring data between the DSP core 1518 and external devices. The processing unit 1532 also includes a battery 1562 mounted in the housing 1526 for supplying electrical power to the processor unit 1532.

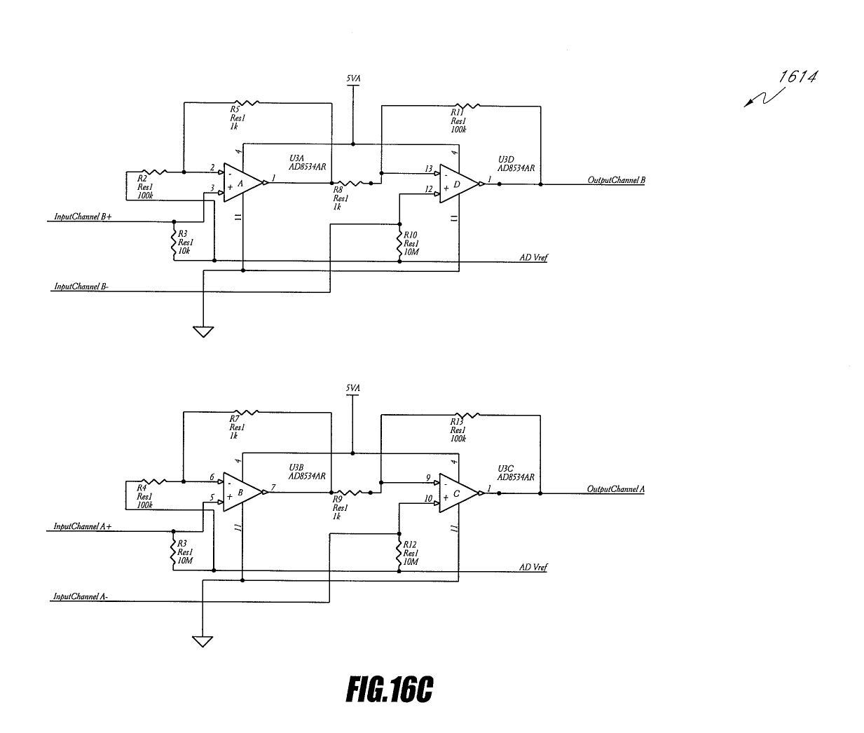

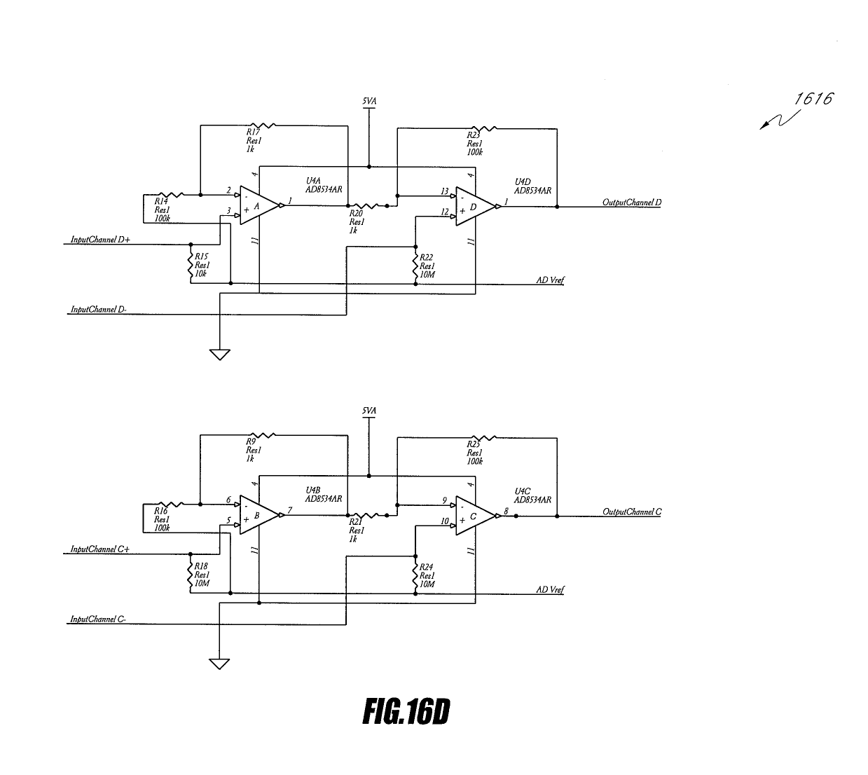

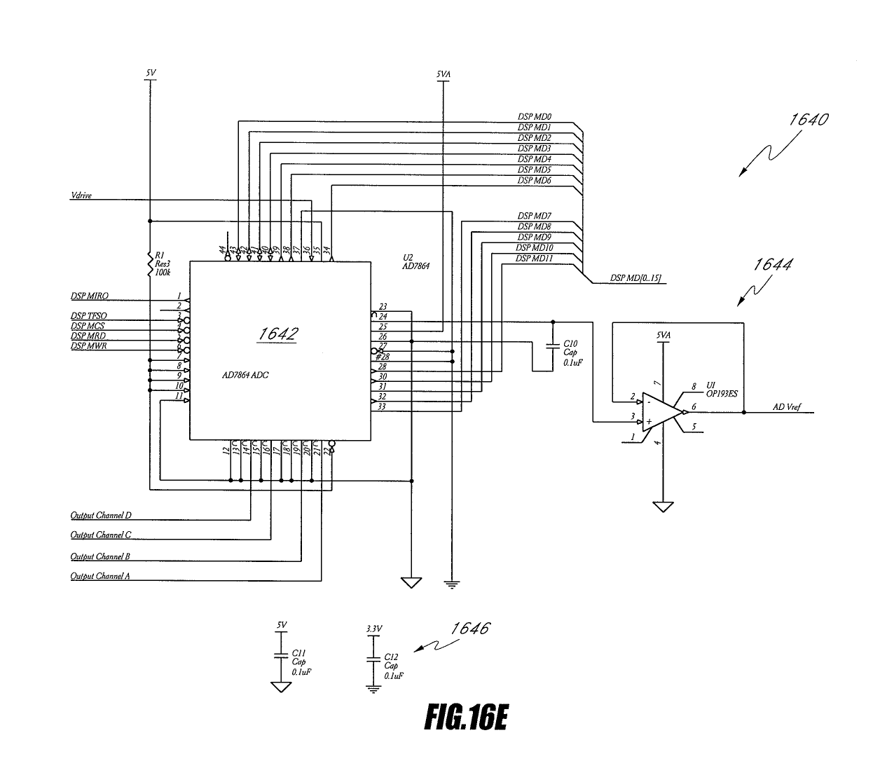

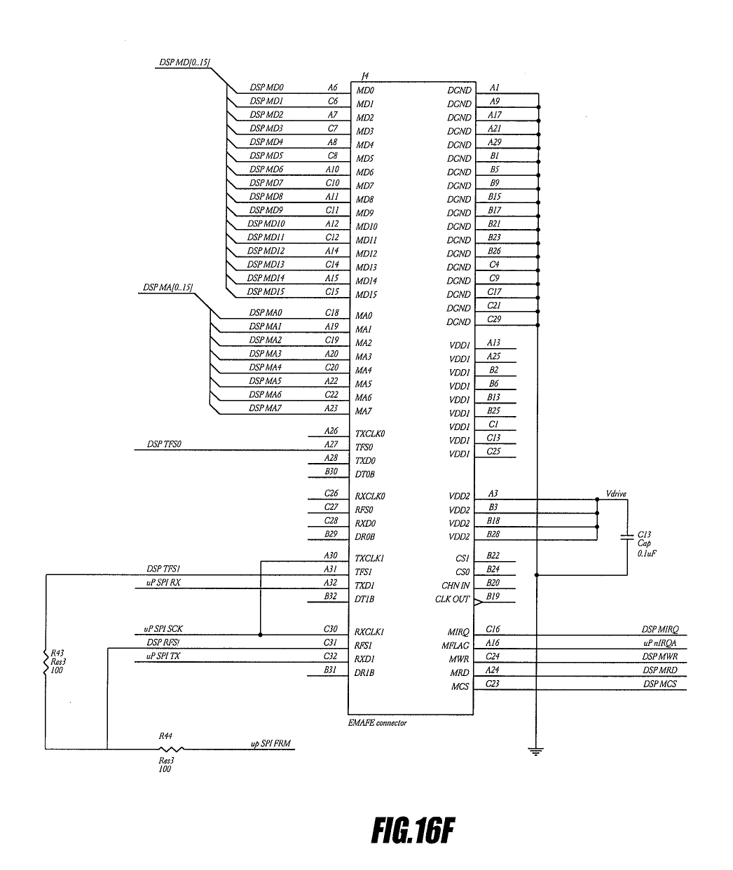

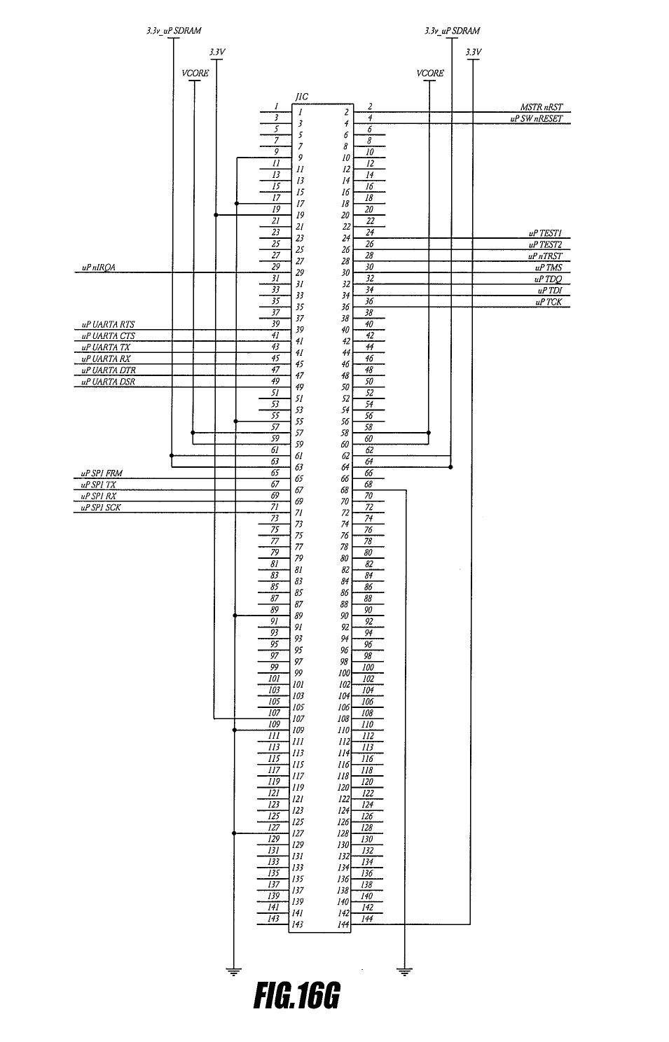

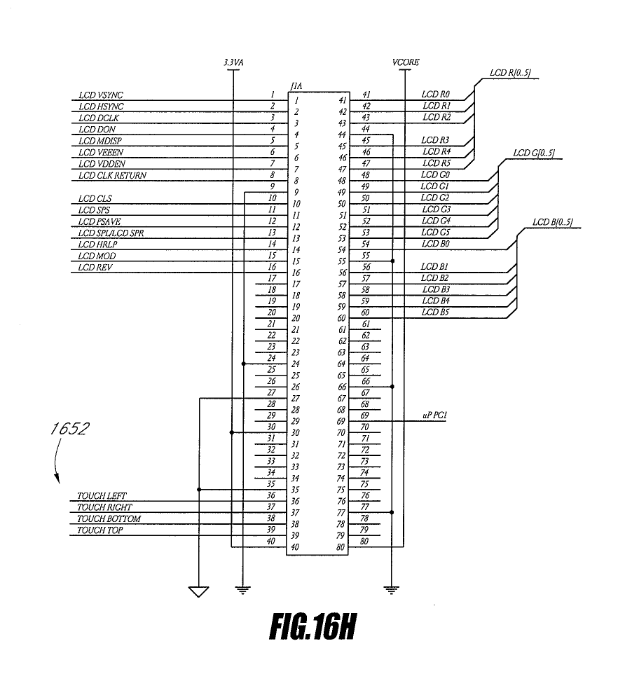

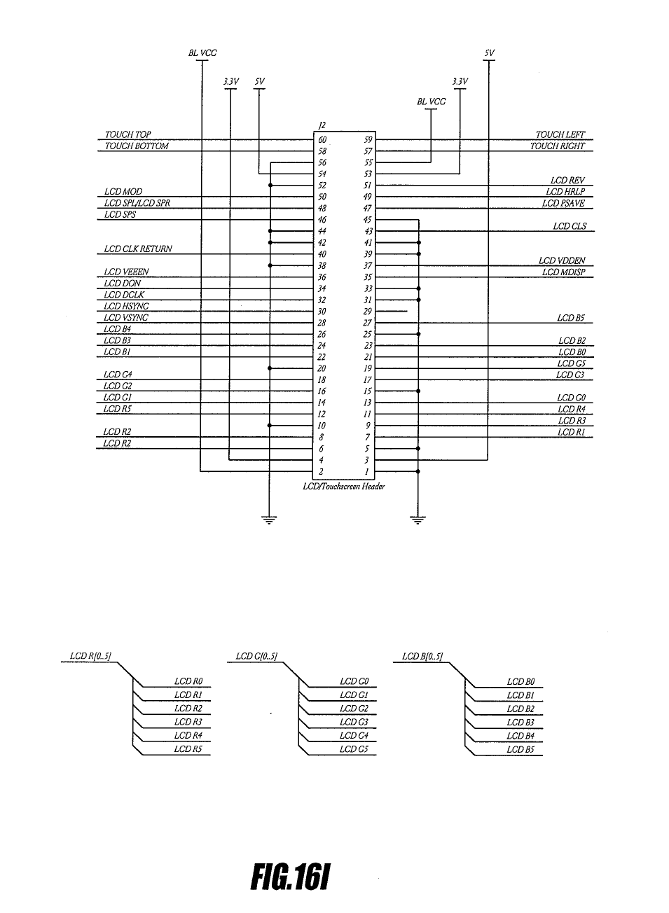

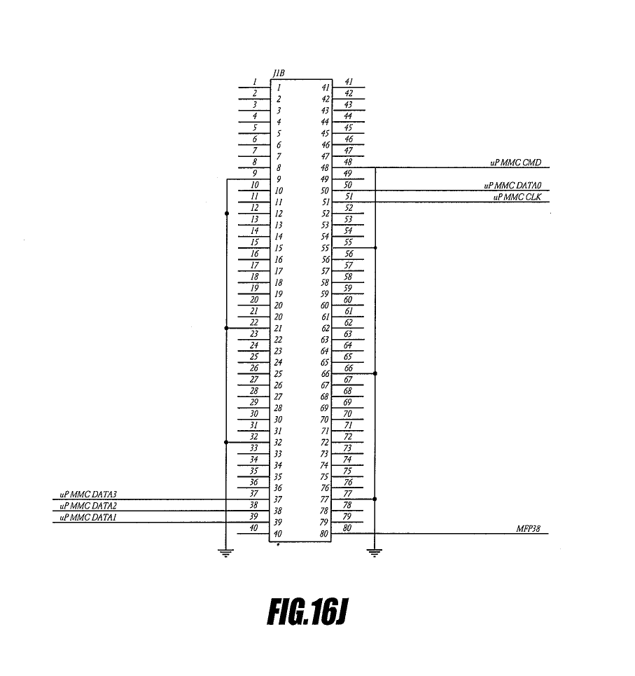

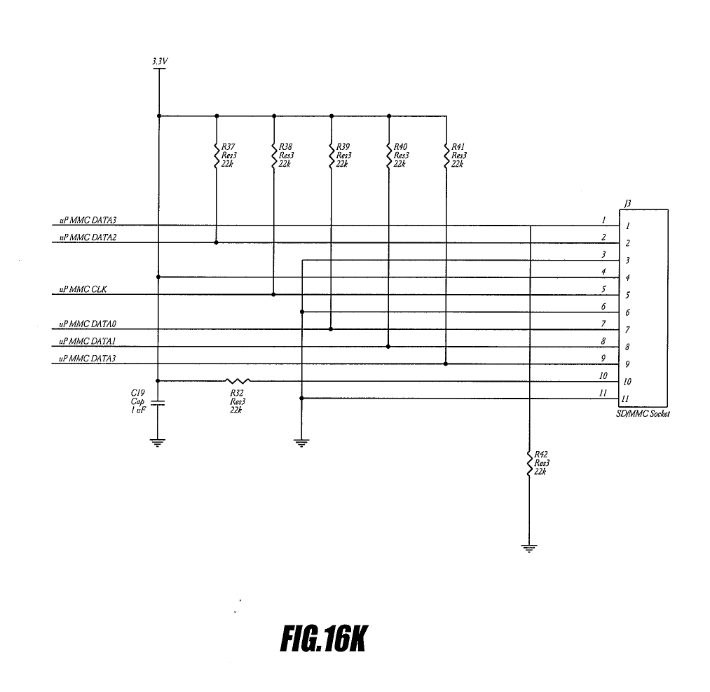

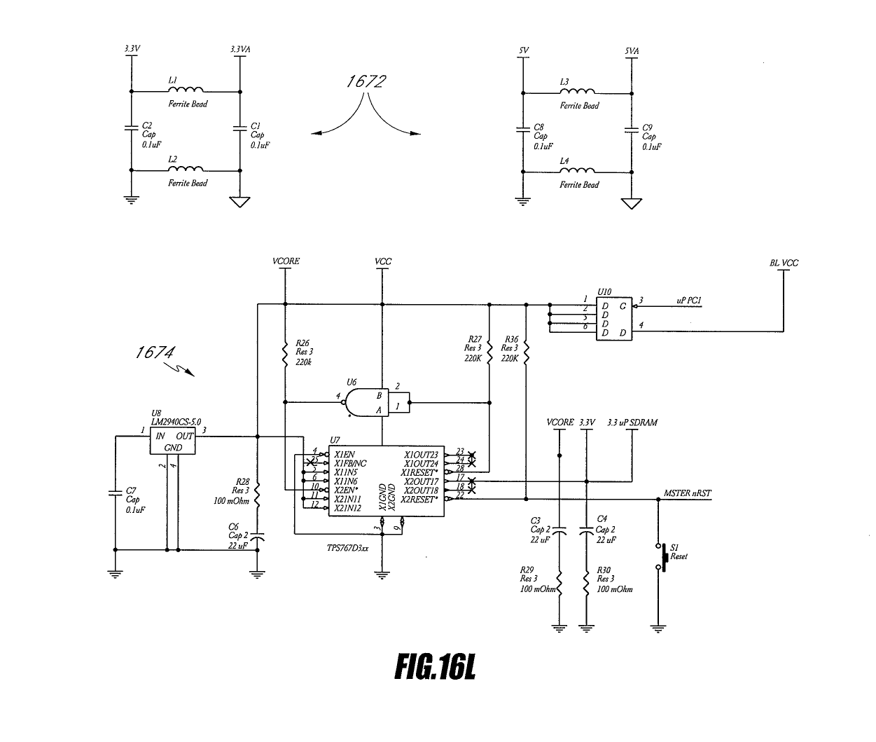

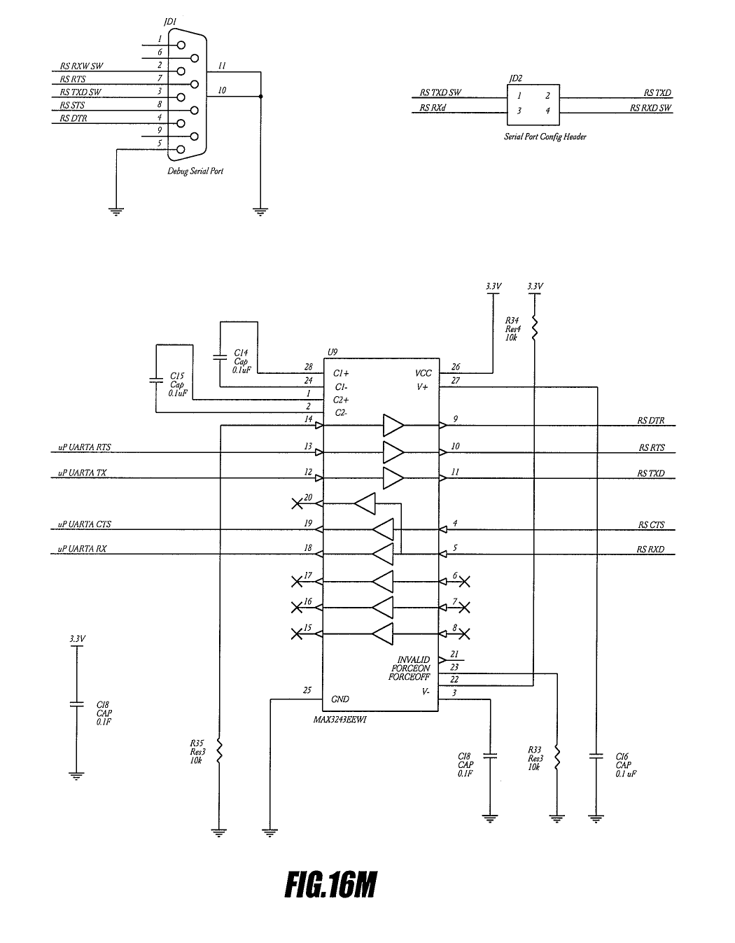

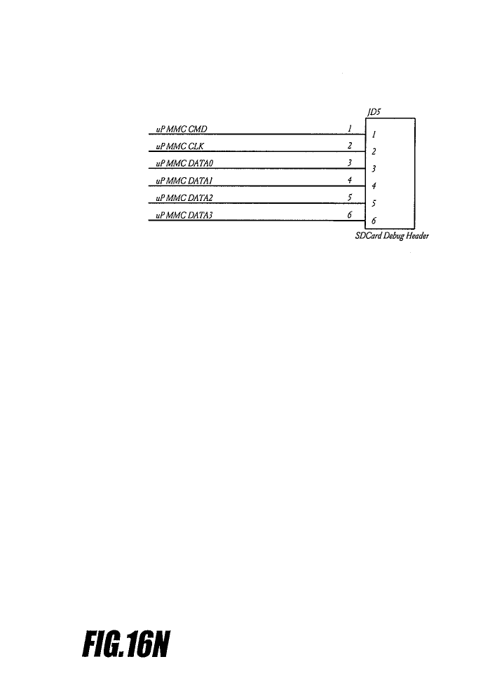

FIGS. 16A-16N are schematic illustrations of the electronics for an embodiment of a device that can function similarly to the device 1410 of FIG. 14A and/or the device 1510 of FIG. 15A and/or the processing unit 1532 shown in FIGS. 15B and 15C.

FIG. 16A schematically illustrates connectors for four leads 1602 coming from acoustic sensors (e.g., sensors 1416A-1416D or sensors 1516A-1516D). These leads can connect the acoustic sensors to a signal conditioner such as the signal conditioners 1430 or 1530, for example. Four channels are shown: channels A-D; however, fewer or more channels may be used in other embodiments.

FIG. 16B schematically illustrates an anti-aliasing filter 1604. The anti-aliasing filter can be part of the signal conditioning portion 1430 of FIG. 14A and/or the analog signal conditioner 1530 of FIG. 15A, for example. The connectors for channels A-D shown in FIG. 16A connect to the connectors for channels A-D in FIG. 16B. This figure shows various electrical connections (solid black lines), capacitors (labeled with C and a capacitance in micro-farads) and resistors (labeled with R and a resistance in ohms). Several ground connections are also shown.

FIG. 16C schematically illustrates differential amplifiers 1614 corresponding to analog input channels A and B. These differential amplifiers have the same gain. The input signal comes into these differential amplifiers 1614 along the “input channels” and is output along the “output channels.” Each differential amplifier is connected to a reference voltage.

FIG. 16D schematically illustrates differential amplifiers 1616 corresponding to analog input channels C and D. These differential amplifiers have the same gain, but a different gain than that of the differential amplifiers 1614 of FIG. 16C. The input signal comes into these differential amplifiers 1616 along the “input channels” and is output along the “output channels.” Each differential amplifier is connected to a reference voltage.

The circuitry of FIGS. 16C and 16D amplifies and/or conditions the signals from the input channels, thus filling, at least in part, the role of the signal conditioning portion 1430 of FIG. 14A and/or the analog signal conditioner 1530 of FIG. 15A.

FIG. 16E schematically illustrates electronics relating to an analog-to-digital converter (ADC) 1640. The ADC 1640 can comprise, for example, the illustrated Analog Devices® AD7864 ADC chip 1642. The inputs to the ADC chip 1642 are labeled “OutputChannel” A-D, because they are the outputs from the differential amplifiers 1614 and 1616 of FIGS. 16C and 16D. The ADC chip 1642 converts the incoming analog signals to digital signals and outputs the digital signals to the DSP, as shown. The ADC 1640 can fill, at least in part, the role of the ADC 1440 of FIG. 14A and/or the analog to digital converter 1540 of FIG. 15A, for example.

A voltage reference buffer 1644 connects to the differential amplifiers 1614 and 1616 of FIGS. 16C and 16D. This buffer 1644 biases the voltage up to 2.5 volts to avoid the need for positive and negative power supplies. FIG. 16E also shows decoupling capacitors 1646.

Neither the DSP (described generally relating to the processor 1450 of FIG. 14A and the digital signal processor 1550 of FIG. 15A) nor the I/O processor (described generally relating to the input/output processor 1460 of FIG. 14A and the input/output processor 1560 of FIG. 15A) is specifically illustrated in the electrical schematics of FIGS. 16A-16N, although several of the components shown are designed to connect with these processors. For example, the I/O processor (not shown) can be an ARM® (Advanced Risc Machine) processor, and the DSP can comprise an EZ-Lite kit, as described above. In certain embodiments, the DSP may comprise a 16, 32, or 64 bit reduced instruction set computer (RISC) device or other suitable microprocessor or computer. The signals from the sensors may be sent through the DSP interface to the ARM processor, which provides interface support to an input device (such as, for example, a touchscreen or keypad), manages data storage on a storage device (such as, for example, a memory card), and also formats information to be displayed on as text or graphics on a display (such as, for example, an LCD monitor).

FIG. 16F is a schematic illustration of an EMAFE connector (such as the connector 1487 of FIG. 14B) that can connect the DSP to the I/O processor.

FIGS. 16G, 16H, and 16J are schematic illustrations of Sharp® LH7A404 card engine connectors that can be used to connect electrical components.

FIG. 16G schematically illustrates the electrical connector that can connect an I/O processor to other components. In some embodiments, this connector can connect the I/O processor to a display, for example.

FIG. 16H schematically illustrates an electrical connector that can connect an I/O processor to other components such as an LCD display with touchscreen. The touchscreen can send signals to an I/O processor through the touch channels 1652 (e.g., the channel labeled “touch left”) if a user touches a portion of the screen (such as the left side, for example).

FIG. 16I schematically illustrates another electrical connector that can connect an I/O processor to other components such as an LCD display with touchscreen. The connectors shown in FIGS. 16H and 16I can help connect, for example, the I/O processor 1460 to the input device 1470 and/or the output device 1490 of FIG. 14A, for example. In some embodiments, the connectors shown in FIGS. 16H and 16I can help connect the I/O processor 1560 to the LCD display with touchscreen 1570 of FIG. 15A.

FIGS. 16J and 16K schematically illustrate electrical connectors that can connect an I/O processor to other components such as a memory card. The connectors shown in FIGS. 16J and 16K can help connect, for example, the I/O processor 1460 to the storage device 1480 of FIG. 14A, for example. In some embodiments, the connectors shown in FIGS. 16J and 16K can help connect the I/O processor 1560 to the removable data card 1580 of FIG. 15A.

FIG. 16L schematically illustrates electronic circuitry relating to power supply. Two power conditioning circuits 1672 can be used to decouple the power supplied to digital circuits from the power supplied to analog circuits. A power sequencing portion 1674 can be used to supply power to various circuits and various portions of processor chips in the correct sequence.

FIGS. 16M and 16N schematically illustrate diagnostic circuits that can connect to various portions of the other circuits described herein for debugging purposes. These diagnostic circuits can be connected to the DSP, for example.

The following table lists examples of electronic components that can advantageously be used in conjunction with the electronic circuitry illustrated in FIGS. 16A-16N.

| | | | | | | | Code | |
|---|---|---|---|---|---|---|---|---|
| | | | | | | | (IEC, | |
| | | | | | | | IPC, | |
| | | | Lib | | Package | | JEDEC, | Part |
| Description | Designator | Footprint | Ref | Model: Footprint | Ref. | Value | JEITA) | Type |
| Capacitor | C1 | CC3216-1206 | Cap | Chip Capacitor; Body | | 0.1 uF | | Cap |
| | | | | 3.2 × 1.6 mm (L × W typ) | | | | |
| Capacitor | C2 | CC3216-1206 | Cap | Chip Capacitor; Body | | 0.1 uF | | Cap |
| | | | | 3.2 × 1.6 mm (L × W typ) | | | | |
| Capacitor | C3 | 3225[1210] | Cap2 | Chip Resistor; Body | | 22 uF | | Cap2 |
| | | | | 3.2 × 2.5 mm (L × W typ) | | | | |
| Capacitor | C4 | 3225[1210] | Cap2 | Chip Resistor; Body | | 22 uF | | Cap2 |
| | | | | 3.2 × 2.5 mm (L × W typ) | | | | |
| Capacitor | C6 | 3225[1210] | Cap2 | Chip Resistor; Body | | 22 uF | | Cap2 |
| | | | | 3.2 × 2.5 mm (L × W typ) | | | | |
| Capacitor | C7 | 3216[1206] | Cap | Chip Resistor; Body | | 1 uF | | Cap |
| | | | | 3.2 × 1.6 mm (L × W typ) | | | | |
| Capacitor | C8 | CC3216-1206 | Cap | Chip Capacitor; Body | | 0.1 uF | | Cap |
| | | | | 3.2 × 1.6 mm (L × W typ) | | | | |
| Capacitor | C9 | CC3216-1206 | Cap | Chip Capacitor; Body | | 0.1 uF | | Cap |
| | | | | 3.2 × 1.6 mm (L × W typ) | | | | |
| Capacitor | C10 | CC3216-1206 | Cap | Chip Capacitor; Body | | 0.1 uF | | Cap |
| | | | | 3.2 × 1.6 mm (L × W typ) | | | | |
| Capacitor | C11 | CC3216-1206 | Cap | Chip Capacitor; Body | | 0.1 uF | | Cap |
| | | | | 3.2 × 1.6 mm (L × W typ) | | | | |
| Capacitor | C12 | CC3216-1206 | Cap | Chip Capacitor; Body | | 0.1 uF | | Cap |
| | | | | 3.2 × 1.6 mm (L × W typ) | | | | |
| Capacitor | C13 | CC3216-1206 | Cap | Chip Capacitor; Body | | 0.1 uF | | Cap |
| | | | | 3.2 × 1.6 mm (L × W typ) | | | | |
| Capacitor | C14 | CC3216-1206 | Cap | Chip Capacitor; Body | | 0.1 uF | | Cap |
| | | | | 3.2 × 1.6 mm (L × W typ) | | | | |
| Capacitor | C15 | CC3216-1206 | Cap | Chip Capacitor; Body | | 0.1 uF | | Cap |
| | | | | 3.2 × 1.6 mm (L × W typ) | | | | |
| Capacitor | C16 | CC3216-1206 | Cap | Chip Capacitor; Body | | 0.1 uF | | Cap |
| | | | | 3.2 × 1.6 mm (L × W typ) | | | | |
| Capacitor | C17 | CC3216-1206 | Cap | Chip Capacitor; Body | | 0.1 uF | | Cap |
| | | | | 3.2 × 1.6 mm (L × W typ) | | | | |
| Capacitor | C18 | CC3216-1206 | Cap | Chip Capacitor; Body | | 0.1 uF | | Cap |
| | | | | 3.2 × 1.6 mm (L × W typ) | | | | |
| Capacitor | C19 | 3216[1206] | Cap | Chip Resistor; Body | | 1 uF | | Cap |
| | | | | 3.2 × 1.6 mm (L × W typ) | | | | |
| Connector | J1A | 2 × 40_HD_Hirose | 2 × 40_HD_Hirose | | | | | DF12 (3.0)- |
| | | | | | | | | 80DS- |
| | | | | | | | | 0.5 V (80) |
| Connector | J1B | 2 × 40_HD_Hirose | 2 × 40_HD_Hirose | | | | | DF12 (3.0)- |
| | | | | | | | | 80DS- |
| | | | | | | | | 0.5 V (80) |
| Connector | J1C | SODIMM_144 | SODIMM_144 | 144-pin SODIMM | | | | |
| Socket | | | | connector | | | | |
| Header, 30- | J2 | HDR2 × 30 | Header | Connector; Header; | | | | LCD/ |
| Pin, Dual | | | 30 × 2 | 30 × 2 Position | | | | Touchscreen |
| row | | | | | | | | Header |
| Connector | J3 | SDCard_CCM05-5761 | CCM05_SD/ | SDCard Socket - ITT | | | | SD/MMC |
| | | | MMC_Socket | Cannon CCM05-5761 | | | | Socket |
| Connector | J4 | 3 × 32_conn | 3 × 32_CONN | 3 rows × 32 pins | | | | EMAFE |
| | | | | connector (EMAFE) | | | | connector |
| Receptacle | JD1 | DSUB1.385-2H9 | D Connector 9 | Connector; D | 788750 | | | Debug |
| Assembly, 9 | | | | Subminiature; 9 | | | | Serial |
| Position, | | | | Position; Right Angle; | | | | Port |
| Right Angle | | | | Pitch 1.385 mm | | | | |
| Header, 2- | JD2 | HDR2 × 2 | Header | Connector; Header; 2 × 2 | | | | Serial |
| Pin, Dual | | | 2 × 2 | Position | | | | Port |
| row | | | | | | | | Config |
| | | | | | | | | Header |
| Header, 10- | JD3 | HDR 2 × 10 | Header | Connector; Header; | | | | JTAG |
| Pin, Dual | | | 10 × 2 | 10 × 2 Position | | | | Header |
| row | | | | | | | | |
| Header, 2- | JD4 | HDR 2 × 2 | Header | Connector; Header; 2 × 2 | | | | Serial |
| Pin, Dual | | | 2 × 2 | Position | | | | Port |
| row | | | | | | | | Config |
| | | | | | | | | Header |
| Header, 6- | JD5 | HDR 1 × 6 | Header 6 | Connector; Header; 6 | | | | SDCard |
| Pin | | | | Position | | | | Debug |
| | | | | | | | | Header |
| Header, 6- | JD6 | HDR 1 × 6 | Header 6 | Connector; Header; 6 | | | | SPI |
| Pin | | | | Position | | | | Debug |
| | | | | | | | | Header |
| Header, 3- | JP1 | HDR 1 × 3H | Header 3H | Connector; Header; 3 | | | | Ch. D |
| Pin, Right | | | | Position; Right Angle | | | | |
| Angle | | | | | | | | |
| Header, 3- | JP2 | HDR 1 × 3H | Header 3H | Connector; Header; 3 | | | | Ch. C |
| Pin, Right | | | | Position; Right Angle | | | | |
| Angle | | | | | | | | |
| Header, 3- | JP3 | HDR 1 × 3H | Header 3H | Connector; Header; 3 | | | | Ch. B |
| Pin, Right | | | | Position; Right Angle | | | | |
| Angle | | | | | | | | |
| Header, 3- | JP4 | HDR 1 × 3H | Header 3H | Connector; Header; 3 | | | | Ch. A |
| Pin, Right | | | | Position; Right Angle | | | | |
| Angle | | | | | | | | |
| Inductor | L1 | INDC2012-0805 | Inductor | Chip Inductor; Body | 0402-A | | IEC: | Ferrite |
| | | | | 2 × 1.2 mm (L × W typ) | | | 1005; | Bead |
| | | | | | | | JEITA: | |
| | | | | | | | 402 | |
| Inductor | L2 | INDC2012-0805 | Inductor | Chip Inductor; Body | 0402-A | | IEC: | Ferrite |
| | | | | 2 × 1.2 mm (L × W typ) | | | 1005; | Bead |
| | | | | | | | JEITA: | |
| | | | | | | | 402 | |
| Inductor | L3 | INDC2012-0805 | Inductor | Chip Inductor; Body | 0402-A | | IEC: | Ferrite |
| | | | | 2 × 1.2 mm (L × W typ) | | | 1005; | Bead |
| | | | | | | | JEITA: | |
| | | | | | | | 402 | |
| Inductor | L4 | INDC2012-0805 | Inductor | Chip Inductor; Body | 0402-A | | IEC: | Ferrite |
| | | | | 2 × 1.2 mm (L × W typ) | | | 1005; | Bead |
| | | | | | | | JEITA: | |
| | | | | | | | 402 | |
| Resistor | R1 | 1608[0603] | Res1 | Chip Resistor; Body | | 10K | | Res1 |
| | | | | 1.6 × 0.8 mm (L × W typ) | | | | |
| Resistor | R2 | 1608[0603] | Res1 | Chip Resistor; Body | | 100K | | Res1 |
| | | | | 1.6 × 0.8 mm (L × W typ) | | | | |
| Resistor | R3 | 1608[0603] | Res1 | Chip Resistor; Body | | 10M | | Res1 |
| | | | | 1.6 × 0.8 mm (L × W typ) | | | | |
| Resistor | R4 | 1608[0603] | Res1 | Chip Resistor; Body | | 100K | | Res1 |
| | | | | 1.6 × 0.8 mm (L × W typ) | | | | |
| Resistor | R5 | 1608[0603] | Res1 | Chip Resistor; Body | | 1K | | Res1 |
| | | | | 1.6 × 0.8 mm (L × W typ) | | | | |
| Resistor | R6 | 1608[0603] | Res1 | Chip Resistor; Body | | 10M | | Res1 |
| | | | | 1.6 × 0.8 mm (L × W typ) | | | | |
| Resistor | R7 | 1608[0603] | Res1 | Chip Resistor; Body | | 1K | | Res1 |
| | | | | 1.6 × 0.8 mm (L × W typ) | | | | |
| Resistor | R8 | 1608[0603] | Res1 | Chip Resistor; Body | | 1K | | Res1 |
| | | | | 1.6 × 0.8 mm (L × W typ) | | | | |
| Resistor | R9 | 1608[0603] | Res1 | Chip Resistor; Body | | 1K | | Res1 |
| | | | | 1.6 × 0.8 mm (L × W typ) | | | | |
| Resistor | R10 | 1608[0603] | Res1 | Chip Resistor; Body | | 10M | | Res1 |
| | | | | 1.6 × 0.8 mm (L × W typ) | | | | |
| Resistor | R11 | 1608[0603] | Res1 | Chip Resistor; Body | | 100K | | Res1 |
| | | | | 1.6 × 0.8 mm (L × W typ) | | | | |
| Resistor | R12 | 1608[0603] | Res1 | Chip Resistor; Body | | 10M | | Res1 |
| | | | | 1.6 × 0.8 mm (L × W typ) | | | | |
| Resistor | R13 | 1608[0603] | Res1 | Chip Resistor; Body | | 100K | | Res1 |
| | | | | 1.6 × 0.8 mm (L × W typ) | | | | |
| Resistor | R14 | 1608[0603] | Res1 | Chip Resistor; Body | | 100K | | Res1 |
| | | | | 1.6 × 0.8 mm (L × W typ) | | | | |
| Resistor | R15 | 1608[0603] | Res1 | Chip Resistor; Body | | 10M | | Res1 |
| | | | | 1.6 × 0.8 mm (L × W typ) | | | | |
| Resistor | R16 | 1608[0603] | Res1 | Chip Resistor; Body | | 100K | | Res1 |
| | | | | 1.6 × 0.8 mm (L × W typ) | | | | |
| Resistor | R17 | 1608[0603] | Res1 | Chip Resistor; Body | | 1K | | Res1 |
| | | | | 1.6 × 0.8 mm (L × W typ) | | | | |
| Resistor | R18 | 1608[0603] | Res1 | Chip Resistor; Body | | 10M | | Res1 |
| | | | | 1.6 × 0.8 mm (L × W typ) | | | | |
| Resistor | R19 | 1608[0603] | Res1 | Chip Resistor; Body | | 1K | | Res1 |
| | | | | 1.6 × 0.8 mm (L × W typ) | | | | |
| Resistor | R20 | 1608[0603] | Res1 | Chip Resistor; Body | | 1K | | Res1 |
| | | | | 1.6 × 0.8 mm (L × W typ) | | | | |
| Resistor | R21 | 1608[0603] | Res1 | Chip Resistor; Body | | 1K | | Res1 |
| | | | | 1.6 × 0.8 mm (L × W typ) | | | | |
| Resistor | R22 | 1608[0603] | Res1 | Chip Resistor; Body | | 10M | | Res1 |
| | | | | 1.6 × 0.8 mm (L × W typ) | | | | |
| Resistor | R23 | 1608[0603] | Res1 | Chip Resistor; Body | | 100K | | Res1 |
| | | | | 1.6 × 0.8 mm (L × W typ) | | | | |
| Resistor | R24 | 1608[0603] | Res1 | Chip Resistor; Body | | 10M | | Res1 |
| | | | | 1.6 × 0.8 mm (L × W typ) | | | | |
| Resistor | R25 | 1608[0603] | Res1 | Chip Resistor; Body | | 100K | | Res1 |
| | | | | 1.6 × 0.8 mm (L × W typ) | | | | |
| Resistor | R26 | C1608-0603 | Res3 | Chip Resistor; Body | | 220K | | Res3 |
| | | | | 1.6 × 0.8 mm (L × W typ) | | | | |
| Resistor | R27 | C1608-0603 | Res3 | Chip Resistor; Body | | 220K | | Res3 |
| | | | | 1.6 × 0.8 mm (L × W typ) | | | | |
| Resistor | R28 | 2012[0805] | Res3 | Chip Resistor; Body | | 100 mOhm | | Res3 |
| | | | | 2.0 × 1.3 mm (L × W typ) | | | | |
| Resistor | R29 | 2012[0805] | Res3 | Chip Resistor; Body | | 100 mOhm | | Res3 |
| | | | | 2.0 × 1.3 mm (L × W typ) | | | | |
| Resistor | R30 | 2012[0805] | Res3 | Chip Resistor; Body | | 100 mOhm | | Res3 |
| | | | | 2.0 × 1.3 mm (L × W typ) | | | | |
| Resistor | R32 | C1608-0603 | Res3 | Chip Resistor; Body | | 22K | | Res3 |
| | | | | 1.6 × 0.8 mm | | | | |
| | | | | (L × W type) | | | | |
| Resistor | R33 | C1608-0603 | Res3 | Chip Resistor; Body | | 10K | | Res3 |
| | | | | 1.6 × 0.8 mm (L × W typ) | | | | |
| Resistor | R34 | C1608-0603 | Res3 | Chip Resistor; Body | | 10K | | Res3 |
| | | | | 1.6 × 0.8 mm (L × W typ) | | | | |
| Resistor | R35 | C1608-0603 | Res3 | Chip Resistor; Body | | 10K | | Res3 |
| | | | | 1.6 × 0.8 mm (L × W typ) | | | | |
| Resistor | R36 | C1608-0603 | Res3 | Chip Resistor; Body | | 220K | | Res3 |
| | | | | 1.6 × 0.8 mm (L × W typ) | | | | |
| Resistor | R37 | C1608-0603 | Res3 | Chip Resistor; Body | | 22k | | Res3 |
| | | | | 1.6 × 0.8 mm (L × W typ) | | | | |
| Resistor | R38 | C1608-0603 | Res3 | Chip Resistor; Body | | 22k | | Res3 |
| | | | | 1.6 × 0.8 mm (L × W typ) | | | | |
| Resistor | R39 | C1608-0603 | Res3 | Chip Resistor; Body | | 22k | | Res3 |
| | | | | 1.6 × 0.8 mm (L × W typ) | | | | |
| Resistor | R40 | C1608-0603 | Res3 | Chip Resistor; Body | | 22k | | Res3 |
| | | | | 1.6 × 0.8 mm (L × W typ) | | | | |
| Resistor | R41 | C1608-0603 | Res3 | Chip Resistor; Body | | 22k | | Res3 |
| | | | | 1.6 × 0.8 mm (L × W typ) | | | | |
| Resistor | R42 | C1608-0603 | Res3 | Chip Resistor; Body | | 22k | | Res3 |
| | | | | 1.6 × 0.8 mm (L × W typ) | | | | |
| Resistor | R43 | C1608-0603 | Res3 | Chip Resistor; Body | | 100 | | Res3 |
| | | | | 1.6 × 0.8 mm (L × W typ) | | | | |
| Resistor | R44 | C1608-0603 | Res3 | Chip Resistor; Body | | 100 | | Res3 |
| | | | | 1.6 × 0.8 mm (L × W typ) | | | | |
| Switch | S1 | EVQ- | SW- | | | | | Reset |
| | | PPFA25 | PB | | | | | |
| Precision, | U1 | SO-8 | OP19 | Small Outline; 8 Leads; | SO-8 | | IPC: | OP193ES |
| Micropower | | | 3ES | Body Width 3.9 mm; | | | SO8; | |
| Operational | | | | Pitch 1.27 mm | | | JEDEC: | |
| Amplifier | | | | | | | MS- | |
| | | | | | | | 012-AA | |
| 4 channel | U2 | MQFP44 | AD786 | | | | | AD7864 |
| Simultaneous | | | 4AS-2 | | | | | |
| Sampling | | | | | | | | |
| A/D | | | | | | | | |
| Low Cost, | U3 | SO-14 | AD853 | Small Outline; 14 | SO-14 | | IPC: | AD8534 |
| 250 mA | | | 4AR | Leads; Body Width 3.9 | | | SO14; | AR |
| Output | | | | mm; Pitch 1.27 mm | | | JEDEC: | |
| Single- | | | | | | | MS- | |
| Supply | | | | | | | 012-AB | |
| Amplifier | | | | | | | | |
| Low Cost, | U4 | SO-14 | AD853 | Small Outline; 14 | SO-14 | | IPC: | AD8534 |
| 250 mA | | | 4AR | Leads; Body Width 3.9 | | | SO14; | AR |
| Output | | | | mm; Pitch 1.27 mm | | | JEDEC: | |
| Single- | | | | | | | MS- | |
| Supply | | | | | | | 012-AB | |
| Amplifier | | | | | | | | |
| TinyLogic | U6 | SC70-6 | NC7S | | | | | |
| Open-Drain | | | Z38 | | | | | |
| NAND Gate | | | | | | | | |

| Dual LDO | U7 | TSSO10x6- | TPS76 | Shrink Small Outline; 28 Leads; Body Width | | | TPS767 |
|---|---|---|---|---|---|---|---|
| Voltage | | G28 | 7D318 | 4.4 mm; Pitch 0.65 mm | | | D3xx |
| Regulator | | | | | | | |
| 1 A Low- | U8 | TS3B | LM294 | D2-PAK; 3 Leads; Body | TS3B | | LM2940 |
| Dropout | | | OCS- | 14.4 (inc. leads) × 10.4 | | | CS-5.0 |
| Regulator | | | 5.0 | mm (L × W max) | | | |
| ±15 kV | U9 | WSO28 | MAX3 | Small Outline; 28 | WSO28 | IPC: | MAX324 |
| ESD- | | | 243EE | Leads; Body Width 7.5 | | SO28W | 3EEWI |
| Protected, | | | WI | mm; Pitch 1.27 mm | | | |
| 1 μA, | | | | | | | |
| 3.0 V/5.5 V, | | | | | | | |
| 250 kbps, | | | | | | | |
| RS-232 | | | | | | | |
| Transceiver | | | | | | | |
| with | | | | | | | |
| AutoShutdown | | | | | | | |
| DSP | | | | Analog Devices 21065L | | | ADDS- |
| Prototyping | | | | EZ-Kit Lite | | | 21065L- |
| Board | | | | | | | EZLITE |

| | | |
|---|---|---|
| I/O | Logic Product Development | CENG- |
| Processing | LH7A404 Card Engine | LH7A404- |
| Unit | | 11-503HC |
| LCD | Logic Product Development 3.5″ | LCD-3.5- |
| Display | Sharp LCD Display Kit | QVGA-10 |
| Panel | | |

The foregoing description of embodiments of the present inventions have been presented for the purpose of illustration and description and are not intended to be exhaustive or to limit the inventions to the form disclosed. Obvious modifications and variations are possible in light of the above disclosure. The embodiments described illustrate the principles of the inventions and practical applications thereof to enable one of ordinary skill in the art to utilize the inventions in various embodiments and with various modifications as suited to the particular use contemplated. The features, steps, and components described herein can be combined, where possible, to form additional embodiments of the disclosed inventions. It is intended that the scope of the inventions be defined by the claims appended hereto.

What is claimed is:

1. A diagnostic device for detecting a location of an occlusion in an artery of a patient, the device comprising:
at least four acoustic sensors, each sensor configured to produce a signal in response to acoustic energy emitted from said artery, each sensor disposed at a determinable position on the patient; and
an analysis module configured to:
receive said signals and said determinable positions;
perform a wavelet transform on each of said signals;
determine from said wavelet transforms at least three relative propagation times of said acoustic energy; and
calculate a location of said occlusion relative to a reference location from said at least three relative propagation times.

2. The diagnostic device of claim 1, wherein said artery is a coronary artery.

3. The diagnostic device of claim 1, wherein each acoustic sensor is responsive to acoustic energy comprising a range from about 300 Hz to about 2000 Hz.

4. The diagnostic device of claim 1, wherein said determinable positions are measured relative to one of said acoustic sensors.

5. The diagnostic device of claim 1, wherein said determinable positions are measured from a template worn by the patient.

6. The diagnostic device of claim 1, wherein said determinable positions are determined using an echo location technique.

7. The diagnostic device of claim 1, wherein said determinable positions are measured with respect to an external reference point.

8. The diagnostic device of claim 1, wherein said reference location corresponds to the determinable position of one of the acoustic sensors.

9. The diagnostic device of claim 1, wherein said at least three relative propagation times are measured relative to one of said acoustic sensors.

10. The diagnostic device of claim 1, wherein each of said wavelet transforms provides wavelet coefficients, and said at least three relative propagation times are determined from said wavelet coefficients.

11. The diagnostic device of claim 10, wherein said at least three relative propagation times are determined from one or more centroids derived from the wavelet coefficients.

12. The diagnostic device of claim 1, wherein said wavelet transform uses a mother wavelet selected from the group consisting of a Haar, a Morlet, a Daubechies, a Hermitian, a Mexican hat, and an orthogonal mother wavelet.

13. The diagnostic device of claim 1, wherein said wavelet transform comprises a discrete wavelet transform.

14. The diagnostic device of claim 1, wherein said analysis module is configured to use a sound speed to calculate said location.

15. The diagnostic device of claim 14, wherein said sound speed is representative of sound propagation in soft body tissue in said patient.

16. The diagnostic device of claim 1, further comprising an anatomical sensor configured to determine an orientation or a location of an anatomical structure in the patient.

17. The diagnostic device of claim 16, wherein said anatomical sensor is selected from the group consisting of an ultrasound device, a magnetic resonance imaging device, an X-ray device, an electrocardiogram device, an electroencephalogram device, and a computer aided tomography device.

18. The diagnostic device of claim 16, wherein said analysis module is configured to receive from said anatomical sensor information related to said orientation or said location, and to determine an anatomical correspondence between said location of said occlusion and said anatomical structure.

19. The diagnostic device of claim 1, further comprising an output module configured to display information related to the location of the occlusion.

20. The diagnostic device of claim 1, further comprising a storage module configured to store information related to said signals, said wavelet transforms, or said location.
